(19) United States
(12) Patent Application Publication
(10) Pub. No.: US 2011/0166173 A1
(43) Pub. Date: Jul. 7, 2011

(54) 3,5-DISUBSTUTUTED 1H-PYRROLO [2,3-B] PYRIDINES AS JNK INHIBITORS

(75) Inventors: Darren Peter Medland, London (GB); Piotr Pawel Graczyk, London (GB); Gurpreet Singh Bhatia, London (GB)

(73) Assignee: Eisai Co., Ltd., Tokyo (JP)

(21) Appl. No.: 10/591,551

(22) PCT Filed: Mar. 3, 2005
(86) PCT No.: PCT/GB2005/000779

§ 371 Date: May 1, 2008

(30) Foreign Application Priority Data
Mar. 5, 2004 (GB) 0405055.5

(51) Int. Cl. A61K 31/437; C07D 471/04; A61P 29/00; A61P 19/00; A61P 31/00; A61P 9/00; A61P 25/28; A61P 25/16; A61P 7/06; A61P 1/04

(52) U.S. Cl. 514/300; 546/113

(57) ABSTRACT

The present invention provides novel compounds of forumula (I) and their use in the inhibition of c-Jun N-terminal kinases. The present invention further provides the use of these compounds in medicine, in particular in the prevention and/or treatment of neurodegenerative disorders related to apoptosis and/or inflammation.

The present invention relates to novel compounds, their use in the inhibition of c-Jun N-terminal kinases, their use in medicine and particularly in the prevention and/or treatment of neurodegenerative disorders related to apoptosis and/or inflammation. The invention also provides processes for manufacture of said compounds, compositions containing them and processes for manufacturing such compositions.

c-Jun N-terminal kinases (hereinafter referred to as “JNKs”) are members of the mitogen-activated protein kinase (MAPK) family. JNKs are involved in response to various stimuli, including proinflammatory cytokines and environmental stress. JNKs, and JNK3 in particular, play an important role during apoptotic death of cells and therefore have been implicated in various disorders including stroke, traumatic brain injury and other neurodegenerative diseases such as Parkinson disease, Alzheimer disease and others. Since JNK activity is a physiological regulator of AP-1 transcriptional activity, JNK inhibitors are expected to reduce inflammatory response.

Apoptosis is a form of cell death in which the cell actively participates in its own destruction in a process involving a characteristic series of biochemical and morphological changes, which are regulated by specific cell death genes. The apoptotic cell death is a process that has been observed in the developing mammalian nervous system. In mice, the inactivation by homologous recombination of genes that encode proteins that promote apoptosis, such as the caspase-3 or the Bax protein, prevents developmental neuronal cell death. The destruction of genes that encode cell death suppressors such as Bcl-x, leads to enhanced neuronal cell death. There is increasing evidence that apoptosis plays an important role in the pathology of acute and chronic neurodegenerative c-Jun is activated by JNKs, which phosphorylate its transcriptional activation domain. In humans there are three JNK genes: JNK1, JNK2 and JNK3. The RNAs encoding JNK1 and JNK2 are expressed in many tissues, including the brain, but JNK3 is restricted to the nervous system and to a smaller extent the heart and testes.

JNKs are strongly activated in cellular responses to various stresses such as UV radiation, heat shock, osmotic shock, DNA-damaging agents, and proinflammatory cytokines such as TNFα, IL-1β, and others. Upstream regulators of the JNK pathway include kinases such as SEK1, MKK7 and MEKK1. There is evidence that Jun kinase activity is required for neuronal apoptosis in vitro. Overexpression of MEKK1 in sympathetic neurones increased c-Jun protein levels and phosphorylation and induced apoptosis in the presence of NGF indicating that activation of the Jun kinase pathway can trigger neuronal cell death. The Jun kinase pathway has been shown to be necessary for the death of differentiated PC12 cells deprived of NGF. Furthermore, compound CEP-1347, which inhibits the c-Jun pathway (upstream of Jun kinase), protects motor neurones against cell death induced by survival factor withdrawal.

In JNK3 homozygous (−/−) knockout mice, epileptic seizures and death of hippocampal CA3 neurones induced by injection of kainic acid is blocked. This indicates that JNK3 is involved in certain forms of neuronal cell death in vivo. It is also a critical component of GluR6-mediated excitotoxicity. Furthermore, JNK3 (−/−) mice appear to develop normally and are viable suggesting that JNK3 is not essential for development or viability.

Strong nuclear JNK3 immunoreactivity in the brain CA1 neurones of patients with acute hypoxia suggests that JNK3 is involved in hypoxia-related diseases. For example, in transgenic mice overexpressing the anti-apoptotic Bcl-2 protein in the nervous system there is a decrease in infarct volume following cerebral ischemia. Similarly, injection of the caspase inhibitor BAF reduces neuronal cell death following hypoxia/ischaemia in neonatal rats. Another example is spinal muscular atrophy (a motor neuron disease) where loss of function mutations in the SMN gene is associated with the disease. Recent data has shown that the wild type SMN protein binds to Bcl-2 and co-operates with it to inhibit apoptosis. These results suggest that inhibitors of neuronal apoptosis could be beneficial in the treatment of human neurodegenerative diseases. There is increasing evidence that neuronal apoptosis is an important pathological feature of stroke, traumatic brain injury and other neurodegenerative diseases. Therefore, pharmacotherapy using inhibitors of neuronal apoptosis may provide a therapeutic benefit in neurodegenerative conditions.

A number of groups have studied the mechanisms of neuronal cell death using in vitro cell culture systems and the results suggest that in some systems the transcription factor c-Jun is activated by the removal of survival signals and promotes cell death.

Antibodies specific for c-Jun protected NGF-deprived rat sympathetic neurones from apoptosis. Analogous neuroprotection due to expression of a c-Jun dominant negative mutant has been demonstrated, whereas overexpression of wild type c-Jun protein was sufficient to induce apoptosis in the presence of NGF. Estus and co-workers recently showed that an increase in c-Jun RNA levels occurs in cortical neurones undergoing apoptosis after treatment with β-amyloid peptide. It has also been shown that c-Jun is required for apoptosis in cerebellar granule neurones deprived of survival signals neurodegeneration. Transient hypoxia may also trigger apoptosis through JNK signaling pathway in developing brain neurones.

Furthermore, JNK3 immunoreactivity is colocalized with Alzheimer disease-affected neurones. Moreover JNK3 is related to neurofibrillary pathology of Alzheimer disease. In particular, JNK3 induces robust phosphorylation of amyloid precursor protein (APP) thus affecting its metabolism in disease state.

The present inventors have provided compounds, which are inhibitors of c-Jun N-terminal kinases.

The first aspect of the invention therefore relates to a compound of formula (I) as illustrated below:

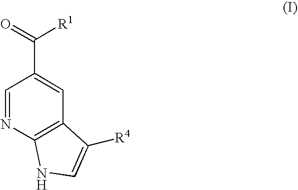

wherein R¹ is an optionally substituted C₃₋₁₂ carbocyclyl or C₃₋₁₂ heterocyclyl group or a group of formula (II)

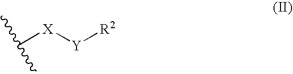

wherein X is NR³, O, S or (CR²²R²²)ₙ, Y is absent or is NR²³, O, or (CR²³R²³)ₙ, R² is optionally substituted C₁₋₁₂ alkyl, C₂₋₁₂ alkenyl, C₁₋₁₂ alkynyl, C₃₋₁₂ carbocyclyl or C₃₋₁₂ heterocyclyl, and R⁴ is an optionally substituted five or six membered heterocyclyl group or an optionally substituted six membered carbocyclyl group;

wherein the optionally substituted carbocyclyl or heterocyclyl group of R¹ is optionally fused to a partially saturated, unsaturated or fully saturated five to seven membered ring containing zero to three heteroatoms, and each substitutable carbon atom in R¹, including the optional fused ring, is optionally and independently substituted by one or more of halogen, C₁₋₁₂ alkyl, C₂₋₁₂ alkenyl, C₂₋₁₂ alkynyl, haloC₁₋₁₂alkyl, C₃₋₁₂ carbocyclyl, C₃₋₁₂ heterocyclyl, (CH₂)ₙOR⁵, (CH₂)ₙNR⁵₂(CH₂)ₙSR⁵, OR⁵, SR⁵, NO₂, CN, NR⁵₂, NR⁵COR⁵, NR⁵CONR⁵₂, NR⁵COR⁵, NR⁵CO₂R⁵, CO₂R⁵, COR⁵, CONR⁵₂, S(O)₂R⁵, SONR⁵₂, S(O)R⁵, SO₂NR⁵₂, or NR⁵S(O)₂R⁵ wherein the C₁₋₁₂ alkyl group optionally contains one or more insertions selected from —O—, —NCR⁵)— —S—, —S(O)— and —S(O₂)—; and each saturated carbon in the optional fused ring is further optionally and independently substituted by ═O, ═S, NNR⁶₂, ═N—OR⁶, ═NNR⁶COR⁶, ═NNR⁶CO₂R⁶, ═NNSO₂R⁶, or ═NR⁶; and each substitutable nitrogen atom in R¹ is optionally substituted by R⁷, COR⁷, SO₂R⁷ or CO₂R⁷;

wherein n is 1 to 6, preferably n is 1, 2 or 3;

wherein R⁵ is hydrogen, C₁₋₁₂ alkyl, C₃₋₁₂ carbocyclyl or C₃₋₁₂ heterocyclyl, optionally substituted by one or more of C₁₋₆ alkyl, C₃₋₁₂ carbocyclyl, C₃₋₁₂ heterocyclyl, halogen, C₁₋₆ haloalkyl, OR⁸, SR⁸, NO₂, CN, NR⁸R⁸, NR⁵COR⁵, NR⁸CONR⁸R⁸, NR⁸COR⁸, NR⁸CO₂R⁸, CO₂R⁸, COR⁸, CONR⁸₂, S(O)₂R⁸, SONR⁸₂, S(O)R⁸, SO₂NR⁸R⁸, NR⁸S(O)₂R⁸, wherein the C₁₋₁₂ alkyl group optionally incorporates one or two insertions selected from the group consisting of —O—, —N(R⁸)—, —S(O)— and —S(O₂)—, wherein each R⁸ may be the same or different and is as defined below;

wherein two R⁵ in NR⁵₂ may optionally form a partially saturated, unsaturated or fully saturated three to seven membered ring containing one to three heteroatoms, optionally and independently substituted by one or more of C₁₋₆ alkyl, halogen, C₁₋₆ haloalkyl, OR⁸, SR⁸, NO₂, CN, NR⁸R⁸, NR⁸COR⁸, NR⁸CONR⁸R⁸, NR⁸COR⁸, NR⁸CO₂R⁸, CO₂R⁸, COR⁸, CONR⁸₂SONR⁸₂, S(O)R⁸, SO₂NR⁸R⁸, NR⁸S(O)₂R⁸,

wherein the C₁₋₆ alkyl group optionally incorporates one or two insertions selected from the group consisting of —O—, —N(R⁸)—, —S(O)— and —S(O₂)—, wherein each R⁸ may be the same or different and is as defined below;

wherein R⁶ is hydrogen, C₁₋₁₂ alkyl, C₃₋₁₂ carbocyclyl or C₃₋₁₂ heterocyclyl, optionally substituted by one or more of C₁₋₆ alkyl, halogen, C₁₋₆ haloalkyl, OR⁸, SR⁸, NO₂, CN, NR⁸R⁸, NR⁸COR⁸, NR⁸CONR⁸R⁸, NR⁸COR⁸, NR⁸CO₂R⁸, CO₂R⁸, COR⁸, CONR⁸₂, S(O)₂R⁸, S(O)R⁸, SO₂NR⁸R⁵, NR⁸S(O)₂R⁸, wherein the C₁₋₁₂ alkyl group optionally incorporates one or two insertions selected from the group consisting of —O—, —N(R⁸)—, —S(O)— and —S(O₂)—, wherein each R⁸ may be the same or different and is as defined below;

wherein R⁷ is hydrogen, C₆₋₁₂ aryl, C₁₋₆ alkyl or C₁₋₆ haloalkyl;

wherein R⁸ is hydrogen, C₁₋₆ alkyl, or C₁₋₆ haloalkyl;

Y is absent or is NR²³, O, or (CR²³R²³)ₙ, wherein each R²³ may be the same or different and is H, C₁₋₄ alkyl, C₁₋₄ alkoxy or C₁₋₄ haloalkyl;

and n is 1 to 6, preferably n is 1, 2, 3 or 4.

R² is C₁₋₁₂ alkyl, C₂₋₁₂ alkenyl, C₂₋₁₂ alkynyl, C₃₋₁₂ carbocyclyl or C₃₋₁₂ heterocyclyl, each of which is optionally substituted, wherein:

the optionally substituted carbocyclyl or heterocyclyl group is optionally fused to one to three unsaturated, partially unsaturated or fully saturated five to seven membered rings containing zero to three heteroatoms;

each substitutable carbon atom in R², including the optional fused ring, is optionally and independently substituted by one or more of C₁₋₁₂ alkyl, C₃₋₁₂ wherein the C₁₋₆ alkyl group optionally incorporates one or two insertions selected from the group consisting of —O—, —N(R¹¹)—, —S(O)— and —S(O₂)—, wherein each R¹¹ may be the same or different and is as defined below;

wherein R¹¹ is hydrogen, C₁₋₆ alkyl, or C₁₋₆haloalkyl;

wherein R⁹ is hydrogen, C₁₋₁₂ alkyl or C₃₋₁₂ aryl, optionally substituted by one or more of C₁₋₄ alkyl, halogen, C₁₋₄ haloalkyl, OR¹², SR¹², NO₂, CN, NR²R¹², NR¹²COR¹², NR¹²CONR¹²R¹², NR¹²COR¹², NR¹²CO₂R¹², CO₁R¹², COR¹², CONR¹²₂, S(O)₂R¹², SONH₂, S(O)R¹², SO₂ NR¹²R¹², NR¹²S(O)₂R¹², wherein the C₁₋₁₂ alkyl group optionally incorporates one or two insertions selected from the group consisting of —O—, —N(R¹²)—, —S(O)— and —S(O₂)—, wherein each R¹² may be the same or different and is as defined below;

wherein R¹⁰ is C₁₋₁₂ alkyl or C₃₋₁₂ aryl, optionally substituted by one or more of C₁₋₄ alkyl, halogen, C₁₋₄ haloalkyl, OR¹², SR¹², NO₂, CN, NR¹²R¹², NR¹²COR¹², NR¹²CONR¹²R¹², NR¹²COR¹², NR¹²CO₂R¹², CO₂R¹², COR¹², CONR¹²₂, S(O)₂R¹², SONH₂, S(O)R¹², SO₂NR¹²R¹², NR¹²S(O)₂R¹², wherein the C₁₋₁₂ alkyl group optionally incorporates one or two insertions selected from the group consisting of —O—, —N(R¹²)—, —S(O)— and —S(O₂)—, wherein each R¹² may be the same or different and is as defined below;

wherein R¹² is hydrogen, C₁₋₄ alkyl, or C₁₋₄ haloalkyl;

X is NR³; O, S or (CR²²R²²))ₙ wherein R²² is independently one or more of halogen, C₁₋₁₂ alkyl, C₂₋₁₂ alkenyl, C₂₋₁₂ alkynyl, C₁₋₁₂ haloalkyl, C₆₋₁₂ carbocyclyl, C₅₋₁₂ heterocyclyl, (CH₂)ₙOR⁵, (CH₂)ₙNR⁵₂, OR⁵, SR⁵, NO₂, CN, NR⁵₂, NR⁵COR⁵, NR⁵CONR⁵₂, NR⁵COR⁵, NR⁵CO₂R⁵, CO₂R⁵, COR⁵, CONR⁵₂, S(O)₂R⁵, SONR⁵₂, S(O)R⁵, SO₂NR⁵₂, or NR⁵S(O)₂R⁵ wherein each R⁵ may be the same or different and is as defined above; and wherein n is 1 to 6, preferably n is 1, 2, 3 or 4;

wherein R³ is a lone electron pair, hydrogen, C₁₋₁₂ alkyl, C₂₋₁₂ alkenyl, C₂₋₁₂ alkynyl, C₃₋₁₂ carbocyclyl or C₃₋₁₂ heterocyclyl, each of which is optionally substituted, wherein:

the optionally substituted carbocyclyl or heterocyclyl group is optionally fused to one to three unsaturated, partially unsaturated or fully saturated five to seven membered rings containing zero to three heteroatoms,

each substitutable carbon atom in R³, including the optional fused ring, is optionally and independently substituted by one or more of C₁₋₁₂ alkyl, C₃₋₁₂ cycloalkyl, C₃₋₁₂ heterocycloalkyl, C₃₋₁₂ aryl, C₃₋₁₂ heteroaryl halogen, C₁₋₁₂ haloalkyl, OR¹³, SR¹³, NO₂, CN, NR¹³R¹³, NR¹³COR¹³, NR¹³CONR¹³R¹³, NR¹³COR¹³, NR¹³CO₂R¹³, CO₂R¹³, COR¹³, CONR¹³R¹³, S(O)₂R¹³, SONH₂, S(O)R¹³, SO₂NR¹³R¹³, NR¹³S(O)₂R¹³, wherein each R¹³ may be the same or different and is as defined above and wherein:

the C₁₋₁₂ alkyl group optionally incorporates one or two insertions selected from the group consisting of —O—, —C(O)—, —N(R¹³)—, —S(O)— and —S(O₂)—, wherein each R¹³ may be the same or different and is as defined above;

the C₁₋₁₂ alkyl, C₃₋₁₂ cycloalkyl, C₃₋₁₂ heterocycloalkyl, C₃₋₁₂ aryl, or C₃₋₁₂ heteroaryl groups are optionally substituted by one or more of halogen, C₁₋₁₂ haloalkyl, OR¹³, SR¹³, NO₂, CN, NR¹³R¹³, NR¹³COR¹³, NR¹³CONR¹³R¹³, NR¹³COR¹³, NR¹³CO₂R¹³, CO₂R¹³, COR¹³, CONR¹³R¹³, S(O)₂R¹³, SONH₂, S(O)R¹³, SO₂NR¹³R¹³, NR¹³S(O)₂R¹³, wherein each R¹³ may be the same or different and is as defined below; and

the C₃₋₁₂ cycloalkyl, C₃₋₁₂ heterocycloalkyl, C₃₋₁₂ aryl, or C₃₋₁₂ heteroaryl groups are optionally substituted by one or more C₁₋₁₂ alkyl groups;

each saturated carbon in R², including the optional fused ring, is further optionally and independently substituted by ═O, ═S, NNR¹³R¹³, ═N—OR¹³, ═NNHCOR¹³, ═NNHCO₂R¹³, ═NNSO₂R¹³, or ═NR¹³, wherein each R¹³ may be the same or different and is as defined below; and

each substitutable nitrogen atom in R³ is optionally substituted by R¹⁴, COR¹³, SO₂R¹³ or CO₂R¹³ wherein each R¹³ and R¹⁴ may be the same or different and is as defined below;

wherein two R¹³ in NR¹³₂ may optionally form a partially saturated, unsaturated or fully saturated three to seven membered ring containing one to three heteroatoms, optionally and independently substituted by one or more of C₁₋₆ alkyl, halogen, C₁₋₆ haloalkyl, OR¹⁵, SR¹⁵, NO₂, CN, NR¹⁵R¹⁵, NR¹⁵COR¹⁵, NR¹⁵CONR¹⁵R¹⁵, NR¹⁵COR¹⁵, NR¹⁵CO₂R¹⁵, CO₂R¹⁵, COR¹⁵, CONR¹⁵₂, S(O)₂R¹⁵, SONR¹⁵₂, S(O)R¹⁵, SO₂NR¹⁵R¹⁵, NR¹⁵S(O)₂R¹⁵,

wherein the C₁₋₆ alkyl group optionally incorporates one or two insertions selected from the group consisting of —O—, —N(R¹⁵)—, —S(O)— and —S(O₂)—, wherein each R¹⁵ may be the same or different and is as defined below;

wherein R¹⁵ is hydrogen, C₁₋₆ alkyl, or C₁₋₆ haloalkyl;

wherein R¹³ is hydrogen, C₁₋₁₂ alkyl or C₃₋₁₂ aryl, optionally substituted by one or more of C₁₋₄ alkyl, halogen, C₁₋₄ haloalkyl, OR¹⁶, SR¹⁶, NO₂, CN, NR¹⁶R¹⁶, NR¹⁶COR¹⁶, NR¹⁶CONR¹⁶R¹⁶, NR¹⁶COR¹⁶ , NR¹⁶CO₂R¹⁶, CO₂R¹⁶, COR¹⁵, CONR¹⁶₂, S(O)₂R¹⁶, SONH₂, S(O)R¹⁶, SO₂NR¹⁶R¹⁶, NR¹⁶S(O)₂R¹⁶, wherein the C₁₋₁₂ alkyl group optionally incorporates one or two insertions selected from the group consisting of —O—, —N(R¹⁶)—, —S(O)— and —S(O₂)—, wherein each R¹⁶ may be the same or different and is as defined below;

wherein R¹⁴ is C₁₋₁₂ alkyl or C₃₋₁₂ aryl, optionally substituted by one or more of C₁₋₄ alkyl, halogen, C₁₋₄ haloalkyl, OR¹⁶, SR¹⁶, NO₂, CN, NR¹⁶R¹⁶, NR¹⁶COR¹⁶, NR¹⁶CONR¹⁶R¹⁶, NR¹⁶COR¹⁶, NR¹⁶CO₂R¹⁶, CO₂R¹⁶, COR¹⁶, CONR¹⁶₂, S(O)₂R¹⁶, SONH₂, S(O)R¹⁶, SO₂NR¹⁶R¹⁶, NR¹⁶S(O)₂R¹⁶, wherein the C₁₋₁₂ alkyl group optionally incorporates one or two insertions selected from the group consisting of —O—, —N(R¹⁶)—, —S(O)— and —S(O₂)—, wherein each R¹⁶ may be the same or different and is as defined below;

wherein R¹⁶ is hydrogen, C₁₋₄ alkyl, or C₁₋₄ haloalkyl;

wherein when X is NR², R² and R³ can form a 3 to 12 membered heterocyclyl ring, more preferably a 5, 6, 7, 8, 9, 10, 11 or 12 membered ring, wherein said ring can be partially saturated, unsaturated or fully saturated containing one to three heteroatoms; wherein the heterocyclylic group formed by R² and R³ can be optionally fused to one to three unsaturated, partially saturated or fully saturated 5 to 7 membered rings and contains from zero to three heteroatoms, any of said rings being optionally and independently substituted with one or more of C₁₋₆ alkyl, halogen, C₁₋₆ haloalkyl, OR²², SR²², NO₂, CN, NR²²R²², NR²²COR²², NR²²CONR²²R²², NR²²COR²², NR²²COR²², CO₂ R²², COR²², CONR²²₂, S(O)₂R²², SONR²²₂, S(O)R²², SO₂NR²²R²², NR²²S(O)₂R²², wherein the C₁₋₆ alkyl group optionally incorporates one or two insertions from —O—, —N(R²²)—, —S(O)— and —S(O₂)— and wherein each R²² may be the same or different;

and wherein R⁴ is a six-membered carbocyclyl group or a five or six-membered heterocyclyl group containing from 1 to 4 heteroatoms independently selected from N, S or O, wherein the optionally substituted six-membered carbocyclyl or five or six-membered heterocyclyl group is optionally fused to a partially saturated, unsaturated or fully saturated five to seven membered ring containing zero to three heteroatoms, and each substitutable carbon or hetero-atom in R⁴ including the optional fused ring, is optionally and independently substituted by one or more of halogen, C₁₋₁₂ alkyl, C₂₋₁₂ alkenyl, C₂₋₁₂ alkynyl, C₁₋₁₂ haloalkyl, C₃₋₁₂ carbocyclyl, C₃₋₁₂ heterocyclyl, (CH₂)ₙOR¹⁷, (CH₂)ₙNR¹⁷₂, OR¹⁷, SR¹⁷, NO₂, CN, NR¹⁷₂, NR¹⁷COR¹⁷, NR¹⁷CONR¹⁷₂, NR¹⁷COR¹⁷, NR¹⁷CO₂^{R}¹⁷, CO₂R¹⁷, COR¹⁷, CONR¹⁷₂, S(O)₂R¹⁷, SONR¹⁷₂, S(O)R¹⁷, SO₂NR¹⁷₂, or NR¹⁷S(O)₂R¹⁷, wherein the C₁₋₁₂ alkyl group optionally contains one or more insertions selected from —O—, —N(R¹²)— —S—, —S(O)— and —S(O₂)—; and each saturated carbon in the optional fused ring is further optionally and independently substituted by ═O, ═S, NNR¹⁸₂, ═N—OR¹⁸, ═NNR¹⁵COR¹⁸, ═NNR¹⁸CO₂R¹⁸, ═NNSO₂R¹⁸, or ═NR¹⁸; and each substitutable nitrogen atom in R⁴ is optionally substituted by R¹⁹, COR¹⁹, SO₂R¹⁹ or CO₂R¹⁹; wherein n is 1 to 6, preferably n is 1, 2 or 3; preferably, wherein each substitutable carbon or hetero-atom in R⁴ is optionally and independently substituted by one or more of C₁₋₆ alkyl, OR²⁰, SR²⁰, NO₂, CN, NR²⁰₂, NR²⁰COR²⁰, NR²⁰CONR²⁰₂, NR²⁰COR²⁰, NHCO₂R²⁰, CO₂R²⁰, COR²⁰, CONR²⁰₂, S(O)₂R²⁰, SONR²⁰₂, S(O)R²⁰, SO₂NR²⁰₂, or NR²⁰S(O)₂R²⁰;

wherein R²⁰ is hydrogen, C₁₋₆ alkyl, or C₁₋₆ haloalkyl;

wherein R¹⁷ is hydrogen, C₁₋₁₂ alkyl, C₃₋₁₂ carbocyclyl or C₃₋₁₂ heterocyclyl, optionally substituted by one or more of C₁₋₆ alkyl, C₃₋₁₂ carbocyclyl, C₃₋₁₂ heterocyclyl, halogen, C₁₋₆ haloalkyl, OR²¹, SR²¹, NO₂, CN, NR²¹R²¹, NR²¹COR²¹, NR¹²CONR²¹R²¹, NR²¹COR²¹, NR²¹CO₂R²¹, CO₂R²¹, COR²¹, CONR²¹₂, S(O)₂R²¹, SONR²¹₂, S(O)R²¹, SO₂NR²¹R²¹, NR²¹S(O)₂R²¹, wherein the C₁₋₁₂ alkyl group optionally incorporates one or two insertions selected from the group consisting of —O—, —N(R²¹)—, —S(O)— and —S(O₂)—, wherein each R²¹ may be the same or different and is as defined below;
wherein two R¹⁷ in NR¹⁷₂ may optionally form a partially saturated, unsaturated or fully saturated three to seven membered ring containing one to three heteroatoms, optionally and independently substituted by one or more of C₁₋₆ alkyl, halogen, C₁₋₆ haloalkyl, OR²¹, SR²¹, NO₂, CN, NR²¹R²¹, NR²¹COR²¹, NR²¹CONR²¹R²¹, NR²¹COR²¹, NR²¹CO₂R²¹, CO₂R²¹, COR²¹, CONR²¹₂, S(O)₂R²¹, SONR²¹₂, S(O)R²¹, SO₂NR²¹R²¹, NR²¹S(O)₂R²¹, wherein the C₁₋₆ alkyl group optionally incorporates one or two insertions selected from the group consisting of —O—, —N(R²¹)—, —S(O)— and —S(O₂)—, wherein each R²¹ may be the same or different and is as defined below;

wherein R¹⁸ is hydrogen, C₁₋₁₂ alkyl, C₃₋₁₂ carbocyclyl or C₃₋₁₂ heterocyclyl, optionally substituted by one or more of C₁₋₆ alkyl, halogen, C₁₋₆ haloalkyl, OR²¹, SR₂₁, NO₂, CN, NR²¹R²¹, NR²¹COR²¹, NR²¹CONR²¹R²¹, NR²¹COR²¹, NR²¹COR²¹, CO₂R²¹, COR²¹, CONR²¹₂, S(O)₂R²¹, S(O)R²¹, SO₂NR²¹R²¹, NR²¹S(O)₂R²¹, wherein the C₁₋₁₂ alkyl group optionally incorporates one or two insertions selected from the group consisting of —O—, —N(R²¹ )—, —S(O)— and —S(O₂)—, wherein each R²¹ may be the same or different and is as defined below;

wherein R¹⁹ is hydrogen, C₆₋₁₂ aryl, C₁₋₆ alkyl or C₁₋₆ haloalkyl;

wherein R²¹ is hydrogen, C₁₋₆ alkyl, or C₁₋₆ haloalkyl;

and the pharmaceutically acceptable salts, and other pharmaceutically acceptable biohydrolyzable derivatives thereof, including esters, amides, carbamates, carbonates, ureides, solvates, hydrates, affinity reagents or prodrugs thereof.

For the avoidance of doubt when a group as defined above contains two or more radicals eg the radical R²¹ as for example in the groups SO₂NR²¹R²¹ and NR³COR³, the two or more radicals i.e. R²¹ may be the same or different.

For the purposes of this invention, alkyl relates to both straight chain and branched alkyl radicals of 1 to 12 carbon atoms, preferably 1 to 8 carbon atoms and most preferably 1 to 4 carbon atoms including but not limited to methyl, ethyl, n-propyl, isopropyl, n-butyl, sec-butyl, isobutyl, tert-butyl n-pentyl, n-hexyl, n-heptyl, n-octyl. In particular, alkyl relates to a group having 1, 2, 3, 4, 5, 6, 7, 8, 9, 10, 11 or 12 carbon atoms. The term alkyl also encompasses cycloalkyl radicals including but not limited to cyclopropyl, cyclobutyl, CH₂-cyclopropyl, CH₂-cyclobutyl, cyclopentyl or cyclohexyl. In particular, cycloalkyl relates to a group having 3, 4, 5, 6, 7, 8, 9, 10, 11 or 12 carbon atoms. Cycloalkyl groups may be optionally substituted or fused to one or more carbocyclyl or heterocyclyl group. Haloalkyl relates to an alkyl radical as defined above preferably having 1 to 8 carbon atoms, preferably 1 to 4 carbon atoms substituted with one or more halide atoms for example one or more of F, Cl, Br or I, such as CH₂CH₂Br, CF₃ or CCl₃.

The term “alkenyl” means a straight chain or branched alkylenyl radical of 2 to 12 carbon atoms, preferably 2 to 6 carbon atoms and most preferably 2 to 4 carbon atoms, and containing one or more carbon-carbon double bonds and includes but is not limited to ethylene, n-propyl-1-ene, n-propyl-2-ene, isopropylene, etc. In particular, alkenyl relates to a group having 2, 3, 4, 5, 6, 7, 8, 9, 10, 11 or 12 carbon atoms. The term “alkynyl” means a straight chain or branched alkynyl radical of 2 to 12 carbon atoms, preferably 2 to 6 carbon atoms and most preferably 2 to 4 carbon atoms, and containing one or more carbon-carbon triple bonds and includes but is not limited to ethynyl, 2-methylethynyl etc. In particular, alkynyl relates to a group having 2, 3, 4, 5, 6, 7, 8, 9, 10, 11 or 12 carbon atoms.

“Carbocyclyl” relates to a saturated, partly unsaturated or unsaturated 3-12 membered hydrocarbon ring preferably a 6-12 membered hydrocarbon ring, including cycloalkyl and aryl.

“Aryl” means an aromatic 3-12 membered hydrocarbon preferably a 6-12 membered hydrocarbon containing one ring or being fused to one or more saturated or unsaturated rings including but not limited to phenyl, napthyl, anthracenyl or phenanthracenyl.

“Heteroaryl” means an aromatic 3-12 membered aryl preferably a 6-12 membered aryl containing one or more heteroatoms selected from N, O or S and containing one ring or being fused to one or more saturated or unsaturated rings and;

“Heterocyclyl” means a 3-12 membered ring system preferably a 6-12 membered ring system containing one or more heteroatoms selected from N, O or S and includes heteroaryl. In particular the terms “carbocyclyl”, “aryl”, “heteroaryl” and “heterocyclyl” relate to a group having 3, 4, 5, 6, 7, 8, 9, 10, 11 or 12 carbon atoms.

The heterocyclyl system can contain one ring or may be fused to one or more saturated or unsaturated rings; the heterocyclyl can be fully saturated, partially saturated or unsaturated and includes but is not limited to heteroaryl and heterocarbocyclyl. Examples of carbocyclyl or heterocyclyl groups include but are not limited to cyclohexyl, phenyl, acridine, benzimidazole, benzofuran, benzothiophene, benzoxazole, benzothiazole, carbazole, cinnoline, dioxin, dioxane, dioxolane, dithiane, dithiazine, dithiazole, dithiolane, furan, imidazole, imidazoline, imidazolidine, indole, indoline, indolizine, indazole, isoindole, isoquinoline, isoxazole, isothiazole, morpholine, napthyridine, oxazole, oxadiazole, oxathiazole, oxathiazolidine, oxazine, oxadiazine, phenazine, phenothiazine, phenoxazine, phthalazine, piperazine, piperidine, pteridine, purine, pyran, pyrazine, pyrazole, pyrazoline, pyrazolidine, pyridazine, pyridine, pyrimidine, pyrrole, pyrrolidine, pyrroline, quinoline, quinoxaline, quinazoline, quinolizine, tetrahydrofuran, tetrazine, tetrazole, thiophene, thiadiazine, thiadiazole, thiatriazole, thiazine, thiazole, thiomorpholine, thianaphthalene, thiopyran, triazine, triazole, and trithiane.

For the purpose of the present invention, the term “fused” includes a polycyclic compound in which one ring contains one or more atoms preferably one, two or three atoms in common with one or more other ring.

Halogen means F, Cl, Br or I, preferably F.

R¹ is preferably a group of formula (II) or an optionally substituted five or six membered carbocyclyl or heterocyclyl group wherein the carbocyclyl or heterocyclyl group is optionally fused to one or more unsaturated rings.

When R¹ is a substituted five or six membered carbocyclyl or heterocyclyl group it is preferably selected from optionally substituted phenyl, acridine, benzimidazole, benzofuran, benzothiophene, benzoxazole, benzothiazole, cyclohexyl furan, imidazole, indole, isoindole, isoquinoline, isoxazole, isothiazole, morpholine, napthaline, oxazole, phenazine, phenothiazine, phenoxazine, piperazine, piperidine, pyrazole, pyridazine, pyridine, pyrrole, quinoline, quinolizine, tetrahydrofuran, tetrazine, tetrazole, thiophene, thiazole, thiomorpholine, thianaphthalene, thiopyran, triazine, triazole or trithiane.

When R¹ is a group of formula (II), X is preferably a group NR³, Y is preferably absent and one or more of R² and R³ are preferably hydrogen alkyl or cycloalkyl, in particular, the group of formula (II) is preferably an alkylamino or cycloalkylamino group preferably selected from optionally substituted methylamino, ethylamino, propylamino, isopropylamino, butylamino, cyclobutylamino, pentylamino, cyclopentylamino, hexylamino, cyclohexylamino, heptylamino, cycloheptylamino, octylamino and cyclooctylamino. In particular, X is an alkylamino or a cycloalkylamino group wherein the alkyl group has 1, 2, 3, 4, 5, 6, 7, 8, 9, 10, 11 or 12 carbon atoms and the cycloalkyl group has 3, 4, 5, 6, 7, 8, 9, 10, 11 or 12 carbon atoms. R¹ may additionally be a group of formula (II) wherein X is NR³ and R² and R³ form a 5, 6, 7 or 8 membered ring, said ring being partially, saturated, fully saturated or unsaturated and optionally substituted as previously discussed.

As discussed above, R¹ can be optionally substituted at any position on the alkylamino, cycloalkyl amino, carbocyclyl, heterocyclyl or optional fused ring.

R¹ is preferably substituted with one or more of OR²⁴, halogen, C₁₋₆ alkyl, C₂₋₆ alkenyl, C₂₋₆ alkynyl, C₁₋₆haloalkyl, C₁₋₆alkylaryl, C₁₋₆alkylheterocyclyl, (CH₂)ₙOR , (CH₂)ₙNR²⁴₂, SR²⁴, NO₂, CN, NR²⁴₂, CO₂R²⁴, NR²⁴C(O)R²⁴, NR²⁴S(O)₂R²⁴, COR²⁴, CONR²⁴₂, S(O)₂R²⁴, S(O)R²⁴ or SO₂NR²⁴₂;

wherein R²⁴ is hydrogen, C₁₋₄ alkyl or C₆₋₁₂ aryl preferably phenyl, or C₅₋₁₂ heterocyclyl preferably pyridine, and n is 1, 2, 3, 4, 5 or 6.

wherein two R²⁴ in NR²⁴₂ may optionally form a partially saturated, unsaturated or fully saturated three to seven membered ring containing one to three heteroatoms, said ring is preferably independently substituted with one or more of halogen, C₁₋₁₂ alkyl, C₂₋₁₂ alkenyl, C₂₋₁₂ alkynyl, C₁₋₁₂ haloalkyl, C₃₋₁₂ carbocyclyl, C₃₋₁₂ heterocyclyl, OR²⁵, SR²⁵, NO₂, CN, NR²⁵₂, NR²⁵COR²⁵, NR²⁵CONR₂₅₂, NR²⁵COR²⁵, NR²⁵CO₂R²⁵, CO₂R²⁵, COR²⁵, CONR²⁵₂, S(O)₂R²⁵, SONR²⁵₂, S(O)R²⁵, SO₂NR²⁵₂, or NR²⁵S(O)₂R²⁵; and each saturated carbon in the optional ring is further optionally and independently substituted by ═O, ═S, NNR²⁶₂, ═N—OR²⁶, ═NNR²⁶COR²⁶, ═NNR²⁶CO₂R²⁶, ═NNSO₂R²⁶, or ═NR²⁶; and each substitutable nitrogen atom is optionally substituted by R²⁷, COR²⁷, SO₂R²⁷ or CO₂R²⁷; wherein R²⁵ is hydrogen, C₁₋₁₂ alkyl, C₆₋₁₂ carbocyclyl or C₅₋₁₂ heterocyclyl, optionally substituted by one or more of C₁₋₆ alkyl, halogen, C₁₋₆ haloalkyl, OR²⁸, SR²⁸, NO₂, CN, NR²⁸R²⁸, NR²⁸COR²⁸, NR²⁸CONR²⁸R²⁸, NR²⁸COR²⁸, NR²⁸CO₂R²⁸, CO₂R²⁸, COR²⁸, CONR²⁸₂, S(O)₂R²⁸, SONR²⁸₂, S(O)R²⁸, SO₂NR²⁸R²⁸, NR²⁸S(O)₂R²⁸, wherein the C₁₋₁₂ alkyl group optionally incorporates one or two insertions selected from the group consisting of —O—, —N(R²⁸)—, —S(O)— and —S(O₂)—, wherein each R²8 may be the same or different and is as defined below;

wherein R is hydrogen, C₁₋₁₂ alkyl, C₆₋₁₂ carbocyclyl or C₅₋₁₂ heterocyclyl, optionally substituted by one or more of C₁₋₆ alkyl, halogen, C₁₋₆ haloalkyl, OR²⁸, SR²⁸, NO₂, CN, NR²⁸R²⁸, NR²⁸COR²⁸, NR²⁸CONR²⁸R²⁸, NR²⁸COR²⁸, NR²⁸CO₂R²⁸, CO₂R²⁸, COR²⁸, CONR²⁸₂, S(O)₂R²⁸, S(O)R²⁸, SO₂NR²⁸R²⁸, NR²⁸S(O)₂R²⁸, wherein the C₁₋₁₂ alkyl group optionally incorporates one or two insertions selected from the group consisting of —O—, —N(R²⁸)—, —S(O)— and —S(O₂)—, wherein each R²⁸ may be the same or different and is as defined below;

wherein R²⁷ is hydrogen, C₁₋₆ alkyl, C₁₋₆ haloalkyl or C₆₋₁₂ aryl;

wherein R²⁸ is hydrogen, C₁₋₆ alkyl, or C₁₋₆ haloalkyl.

R⁴ is preferably selected from phenyl, cyclohexyl, acridine, benzimidazole, benzofuran, benzothiophene, benzoxazole, benzothiazole, indole, isoindole, indolizine, indazole, isoindole, isoquinoline, morpholine, napthalene, phenazine, phenothiazine, phenoxazine, piperazine, piperidine, pyridazine, pyridine, pyridinyl, pyrimidinyl, pyrazinyl, pyridazinyl, quinoline, quinolizine, tetrazine, thiomorpholine, thianaphthalene, thiopyran, triazine, trithiane, furan, imidazole, isoxazole, isothiazole, oxazole, oxadiazole, oxathiazole, pyrazole, pyrrole, tetrazole, thiophene, thiadiazole, thiatriazole, thiazole or triazole.

As discussed above, R⁴ can be optionally substituted at any position on the carbocyclyl, heterocyclyl or optional fused ring. Preferably, each substitutable carbon or hetero-atom in R⁴ is optionally and independently substituted by one or more of C₁₋₆ alkyl, OR²⁰, SR²⁰, NO₂, CN, NR²⁰₂, NR²⁰COR²⁰, NR²⁰CONR²⁰₂, NR²⁰COR²⁰, NHCO₂R²⁰, CO₂R²⁰, COR²⁰, CONR²⁰₂, S(O)₂R²⁰, SONR²⁰₂, S(O)R²⁰, SO₂NR²⁰₂, or NR²⁰S(O)₂R²⁰;

wherein R²⁰ is hydrogen, C₁₋₆ alkyl, or C₁₋₆ haloalkyl.

When R⁴ is a six-membered carbocyclyl or heterocyclyl group, R⁴ is preferably substituted with one or more of OR²⁹, NR²⁹₂, SR²⁹, (CH₂)ₙOR²⁹, (CH₂)ₙNR²⁹₂, halogen, C₁₋₆ alkyl, C₂₋₆ alkenyl, C₂₋₆ alkynyl, haloalkyl, NO₂, CN, NR²⁹C(O)R²⁹, NR²⁹S(O)₂R²⁹, CO₂R²⁹, COR²⁹, CONR²⁹₂, S(O)₂R²⁹, S(O)R²⁹ or SO₂NR²⁹₂;

wherein R²⁹ is hydrogen, C₁₋₄ alkyl, C₅₋₁₂ heterocyclyl or C₆₋₁₂ aryl preferably phenyl, and n is 1, 2, 3, 4, 5 or 6.

wherein two R²⁹ in NR²⁹₂ may optionally form a partially saturated, unsaturated or fully saturated five to seven membered ring containing one to three heteroatoms, optionally and independently substituted with one or more of halogen, C₁₋₁₂ alkyl, C₂₋₁₂ alkenyl, C₂₋₁₂ alkynyl, C₁₋₁₂ haloalkyl, C₆₋₁₂ carbocyclyl, C₅₋₁₂ heterocyclyl, OR³⁰, SR³⁰, NO₂, CN, NR³⁰₂, NR³⁰COR³⁰, NR³⁰CONR³⁰₂, NR³⁰COR³⁰, NR³⁰CO₂R³⁰, CO₂R³⁰, COR³⁰, CONR³⁰₂, S(O)₂R³⁰, SONR³⁰₂, S(O)R³⁰, SO₂NR³⁰₂, or NR³⁰S(O)₂R³⁰; and each saturated carbon in the optional ring is further optionally and independently substituted by ═O, ═S, NNR³¹₂, ═N—OR³¹, ═NNR³¹COR³¹, ═NNR³¹CO₂R³¹, ═NNSO₂R³¹, or ═NR³¹, and each substitutable nitrogen atom is optionally substituted by R³², COR³², SO₂R³² or CO₂R³²;

wherein R³⁰ is hydrogen, C₁₋₁₂ alkyl, C₆₋₁₂ carbocyclyl or C₅₋₁₂ heterocyclyl, optionally substituted by one or more of C₁₋₆ alkyl, halogen, C₁₋₆ haloalkyl, OR³³, SR³³, NO₂, CN, NR³³R³³, NR³³COR³³, NR³³CONR³³R³³, NR³³COR³³, NR³³CO₂R³³, CO₂ R³³, CR³³, CONR³³₂, S(O)₂R³³, SONR³³₂, S(O)R³³, SO₂NR³³R³³, NR³³S(O)₂R³³, wherein the C₁₋₁₂ alkyl group optionally incorporates one or two insertions selected from the group consisting of —O—, —N(R³³)—, —S(O)— and —S(O₂)—, wherein each R³³ may be the same or different and is as defined below;

wherein R³¹ is hydrogen, C₁₋₁₂ alkyl, C₆₋₁₂ carbocyclyl or C₅₋₁₂ heterocyclyl, optionally substituted by one or more of C₁₋₆ alkyl, halogen, C₁₋₆ haloalkyl, OR³³, SR³³, NO₂, CN, NR³³R³³, NR³³COR³³, NR³³CONR³³R³³, NR³³COR³³, NR³³CO₂R³³, CO₂ R³³, COR³³, CONR³³₂, S(O)₂R³³, S(O)R³³, SO₂Nr³³R³³, NR³³S(O)₂R³³, wherein the C₁₋₁₂ alkyl group optionally incorporates one or two insertions selected from the group consisting of —O—, —N(R³³)—, —S(O)— and —S(O₂)—, wherein each R²¹ may be the same or different and is as defined below;

wherein R³² is hydrogen, C₆₋₁₂ aryl, C₁₋₆ alkyl or C₁₋₆ haloalkyl;

wherein R³³ is hydrogen, C₁₋₆ alkyl, or C₁₋₆ haloalkyl.

When R⁴ is a five-membered heterocyclyl, it is preferably a group

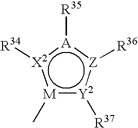

Wherein A, X², Y² or Z are independently selected from N, O, C, S and M is C or N, wherein one, two, three or four of A, X², Y², Z and M is other than C, preferably R⁴ is furan, imidazole, isoxazole, isothiazole, oxazole, oxadiazole, oxatriazole, pyrazole, pyrrole, tetrazole, thiophene, thiadiazole, thiatriazole, thiazole or triazole;

R³⁴, R³⁵, R³⁶ or R³⁷ are independently selected from a lone electron pair, hydrogen, halogen, C₁₋₁₂ alkyl, C₁₋₁₂ haloalkyl, OR³⁸, SR³⁸, NO₂, CN, NR³⁸₂, NR³⁸COR³⁸, NR³⁸CONR³⁸₂, NR³⁸COR³⁸, NR³⁸CO₂R³⁸, (CH₂)ₙOR³⁸, (CH₂)ₙNR³⁸₂, CO₂R³⁸, COR³⁸, CONR³⁸₂, S(O)₂R³⁸, SONR³⁸₂, S(O)R³⁸, SO₂NR³⁸₂, or NHS(O)₂R³⁸;

wherein n is 1 to 6, preferably n is 1, 2 or 3;

or wherein any two of R³⁴, R³⁵, R³⁶ or R³⁷ may optionally form a partially saturated, unsaturated or fully saturated five to seven membered ring containing zero to three heteroatoms, each saturated carbon in the optional fused ring is further optionally and independently substituted with one or more of halogen, C₁₋₁₂ alkyl, C₂₋₁₂ alkenyl, C₂₋₁₂ alkynyl, C₁₋₁₂ haloalkyl, C₆₋₁₂ carbocyclyl, C₅₋₁₂ heterocyclyl, OR³⁸, SR³⁸, NO₂, CN, NR³⁸₂, NR³⁸CONR³⁸₂, NR³⁸COR³⁸, NR³⁸CO₂R³⁸, (CH₂)ₙOR³⁸, (CH₂)ₙNR³⁸₂, CO₂R³⁸, COR³⁸, CONR³⁸₂, S(O)R³⁸, SONR³⁸₂, S(O)R³⁸, SO₂NR³⁸₂, or NR³⁸S(O)₂R³⁸; and each saturated carbon in the optional fused ring is further optionally and independently substituted by ═O, ═S, NNR³⁹₂, ═N—OR³⁹, ═NNR³⁹COR³⁹, ═NNR³⁹CO₂R³⁹, ═NNSO₂R³⁹, or ═NR³⁹; and each substitutable nitrogen atom in R⁴ is optionally substituted by R⁴⁰, COR⁴⁰, SO₂R⁴⁰ or CO₂R⁴⁰;

wherein n is 1 to 6, preferably n is 1, 2 or 3;

wherein R³⁸ is hydrogen, C₁₋₁₂ alkyl, C₆₋₁₂ carbocyclyl or C₅₋₁₂ heterocyclyl, optionally substituted by one or more of C₁₋₆ alkyl, halogen, C₁₋₆ haloalkyl, OR⁴¹, SR⁴¹, NO₂, CN, NR⁴¹R⁴¹, NR⁴¹CONR⁴¹R⁴¹, NR⁴¹COR⁴¹, N⁴¹CO₂R⁴¹, CO₂R⁴¹, COR⁴¹, CONR⁴¹₂, S(O)₂R⁴¹, SONR⁴¹₂, S(O)R⁴¹, SO₂NR⁴¹R⁴¹, NR⁴¹S(O)₂R⁴¹, wherein the C₁₋₁₂ alkyl group optionally incorporates one or two insertions selected from the group consisting of —O—, —N(R⁴¹)—, —S(O)— and —S(O₂)—, wherein each R⁴¹ may be the same or different and is as defined below;

wherein R³⁹ is hydrogen, C₁₋₁₂ alkyl, carbocyclyl or heterocyclyl, optionally substituted by one or more of C₁₋₆ alkyl, halogen, C₁₋₆ haloalkyl, OR⁴¹, SR⁴¹, NO₂, CN, NR⁴¹R⁴¹, NR⁴¹COR⁴¹, NR⁴¹CONR⁴¹R⁴¹, NR⁴¹COR⁴¹, NR⁴¹CO₂R⁴¹, CO₂R⁴¹, COR⁴¹, CONR⁴¹₂, S(O)₂R⁴¹, S(O)R⁴¹, SO₂NR⁴¹R⁴¹, NR⁴¹S(O)₂R⁴¹, wherein the C₁₋₁₂ alkyl group optionally incorporates one or two insertions selected from the group consisting of —O—, —N(R⁴¹)—, —S(O)— and —S(O₂)—, wherein each R⁴¹ may be the same or different and is as defined below;

wherein R⁴⁰ is hydrogen, C₁₋₆ alkyl, C₁₋₆ haloalkyl or C₆₋₁₂ aryl.

wherein R⁴¹ is hydrogen, C₁₋₆ alkyl, or C₁₋₆ haloalkyl.

More preferably R³⁴, R³⁵, R³⁶ or R³⁷ are independently selected from a lone electron pair, hydrogen, halogen, C₁₋₆ alkyl, C₁₋₆ haloalkyl, OR⁴², SR⁴², CN, NR⁴²₂, NR⁴²COR⁴², CO₂R⁴², COR⁴², CONR₄₂, S(O)₂R⁴², or S(O)R⁴²;

wherein R⁴² is hydrogen, C₁₋₄ alkyl, preferably methyl or ethyl or carbocyclyl, preferably phenyl.

Representative compounds according to the first aspect of the invention are illustrated below.

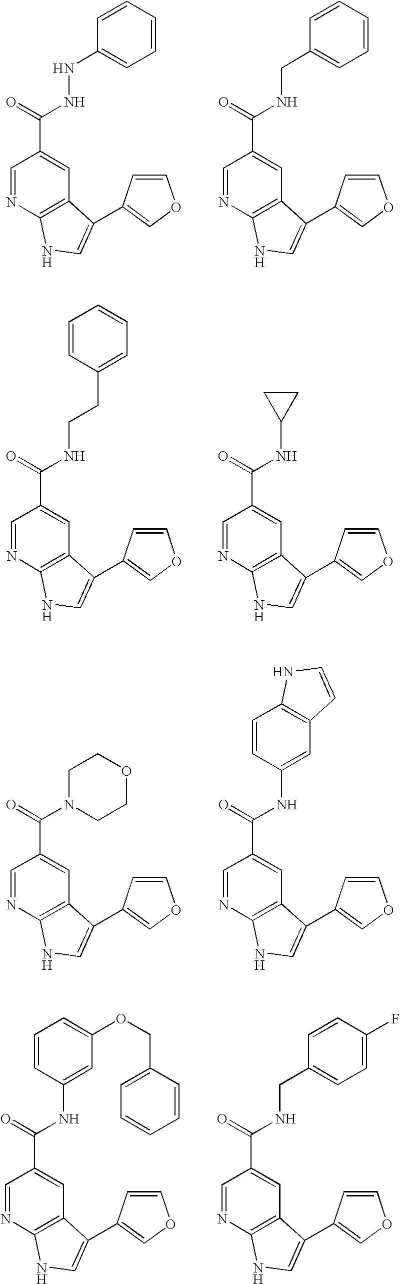

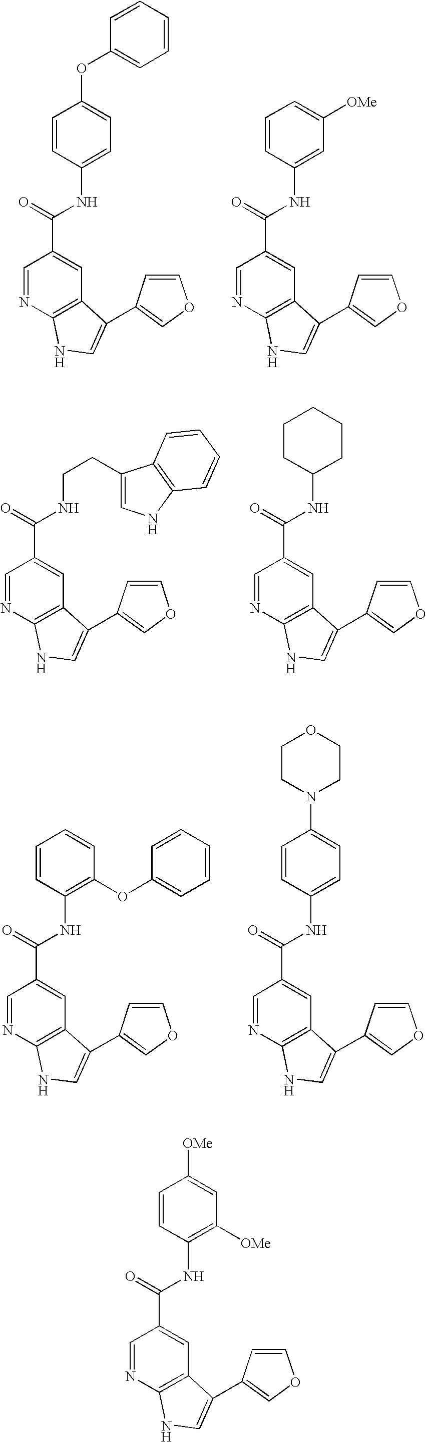

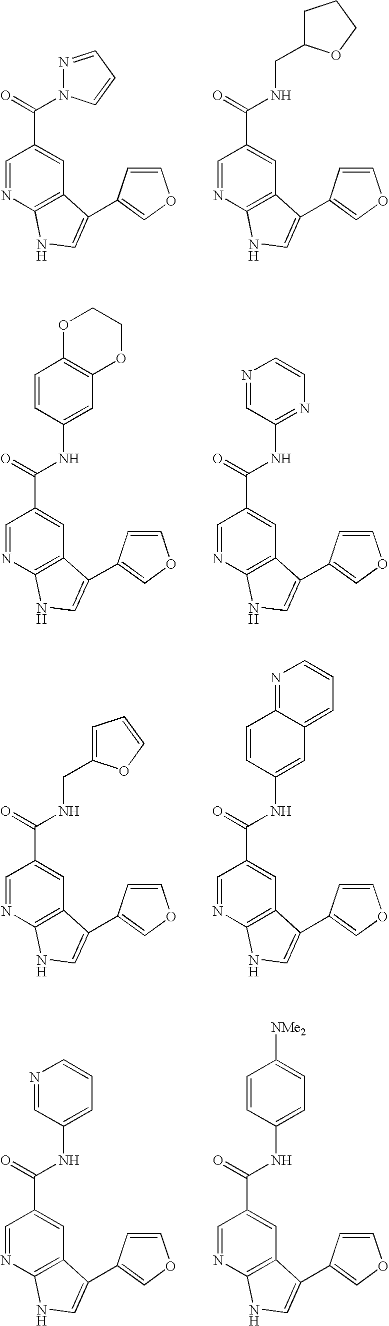

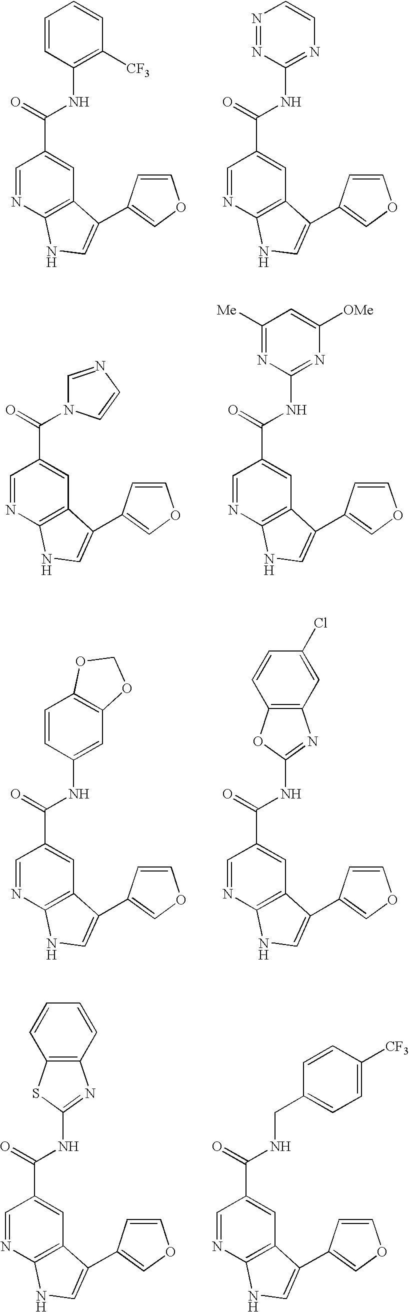

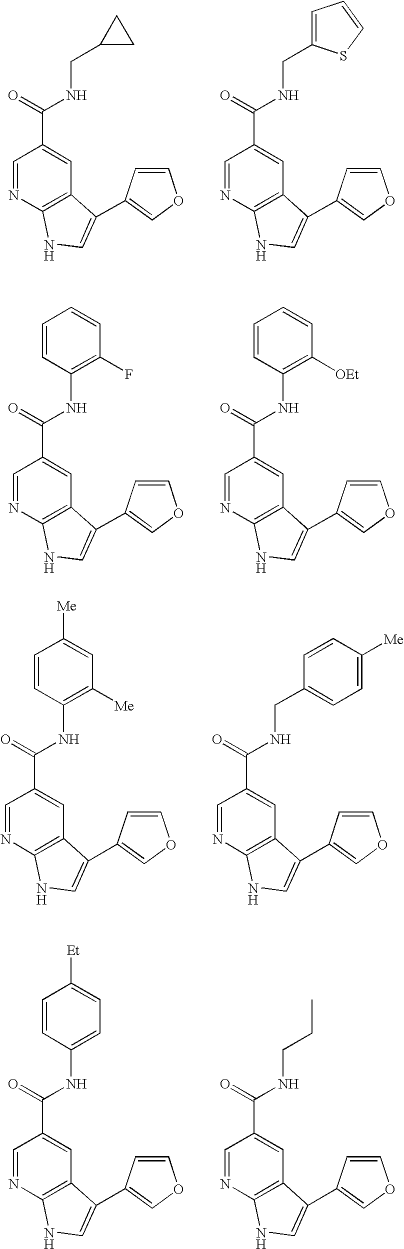

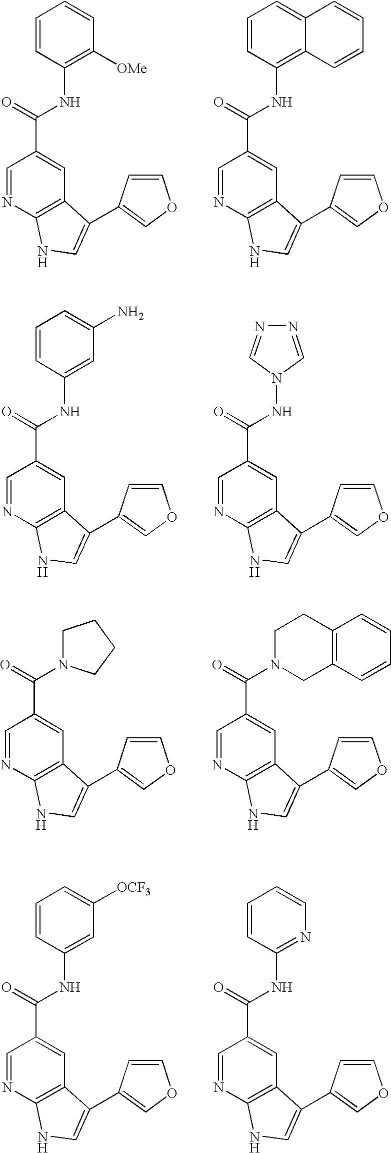

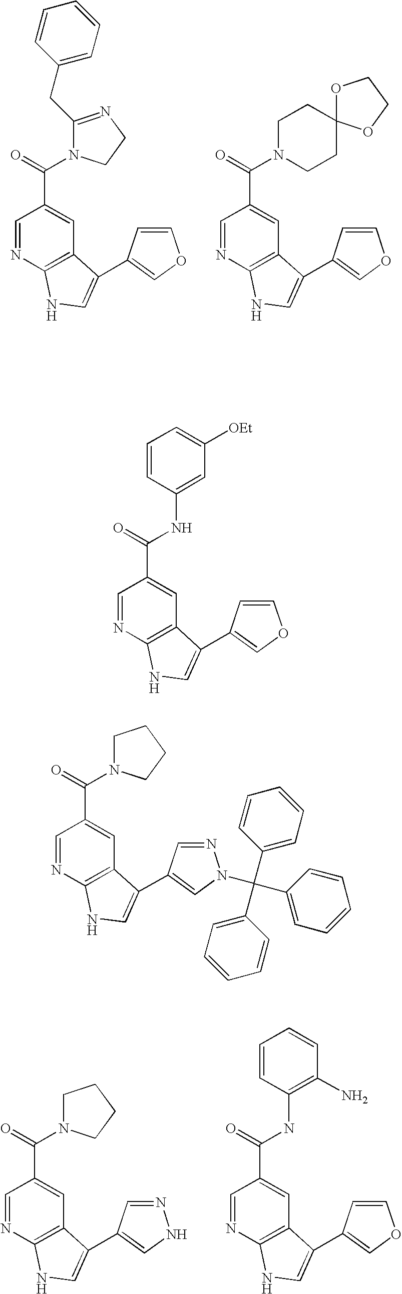

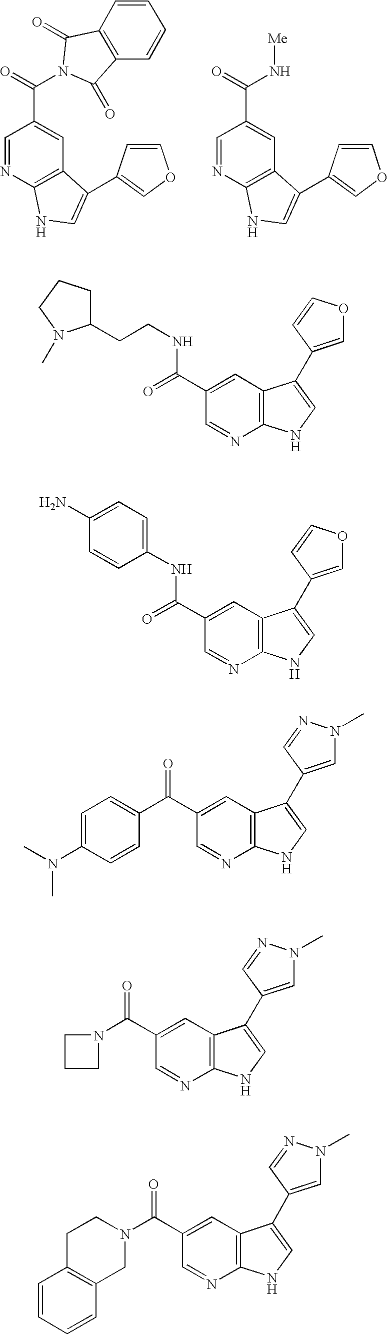

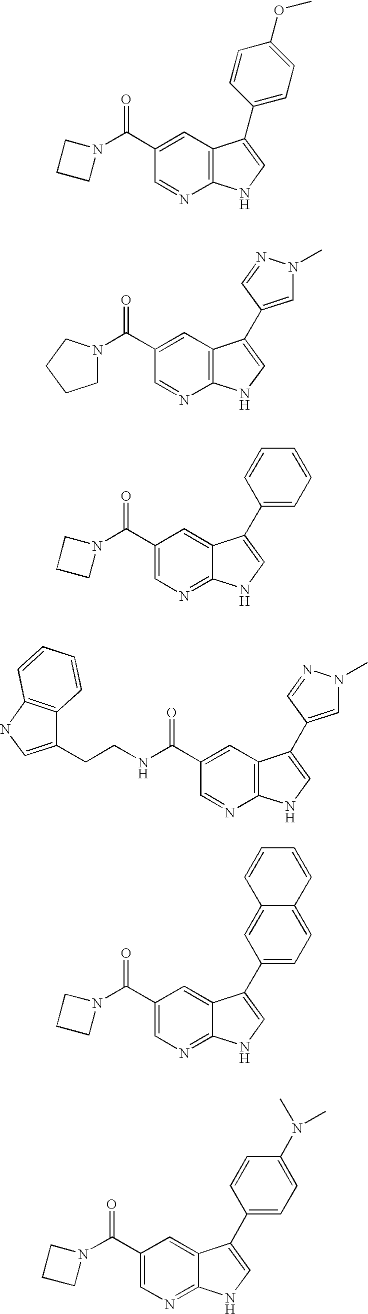

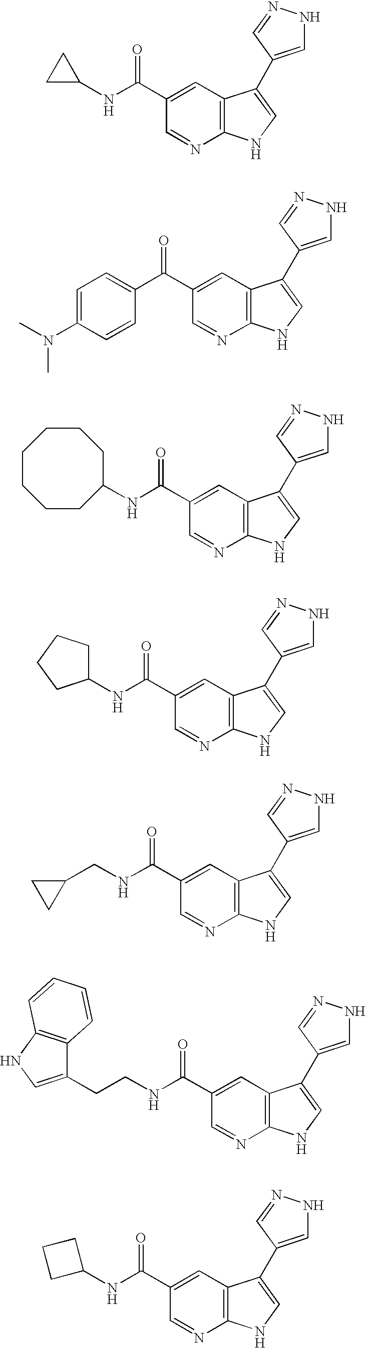

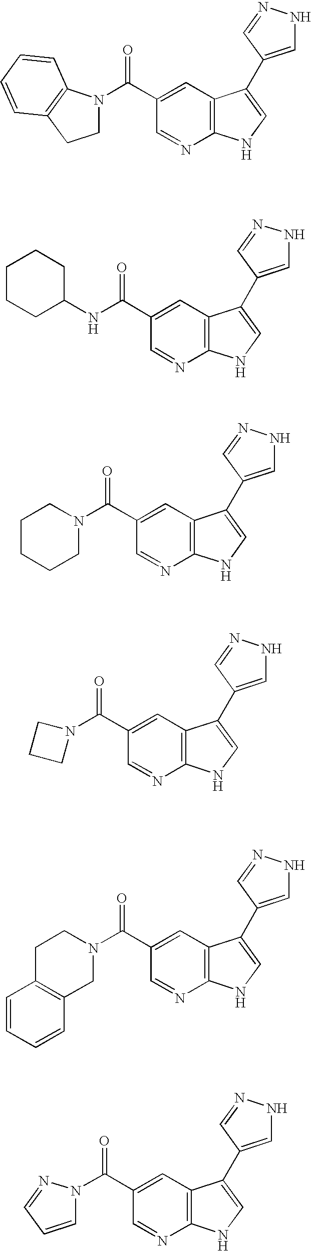

The compounds of the first aspect may be provided as a salt, preferably as a pharmaceutically acceptable salt of compounds of formula (I). Examples of pharmaceutically acceptable salts of these compounds include those derived from organic acids such as acetic acid, malic acid, tartaric acid, citric acid, lactic acid, oxalic acid, succinic acid, fumaric acid, maleic acid, benzoic acid, salicylic acid, phenylacetic acid, mandelic acid, methanesulphonic acid, benzenesulphonic acid and p-toluenesulphonic acid, mineral acids such as hydrochloric and sulphuric acid and the like, giving methanesulphonate, benzenesulphonate, p-toluenesulphonate, hydrochloride and sulphate, and the like, respectively or those derived from bases such as organic and inorganic bases. Examples of suitable inorganic bases for the formation of salts of compounds for this invention include the hydroxides, carbonates, and bicarbonates of anumonia, lithium, sodium, calcium, potassium, aluminium, iron, magnesium, zinc and the like. Salts can also be formed with suitable organic bases. Such bases suitable for the formation of pharmaceutically acceptable base addition salts with compounds of the present invention include organic bases, which are nontoxic and strong enough to form salts. Such organic bases are already well known in the art and may include amino acids such as arginine and lysine, mono-, di-, or trihydroxyalkylamines such as mono-, di-, and triethanolamine, choliae, mono-, di-, and trialkylamines, such as methylamine, dimethylamine, and trimethylamine, guanidine; N-methylglucosamine; N-methylpiperazine; morpholine; ethylenediamine; N-benzylphenethylamine; tris(hydroxymethyl) aminomethane; and the like.

Salts may be prepared in a conventional manner using methods well known in the art. Acid addition salts of said basic compounds may be prepared by dissolving the free base compounds according to the first aspect of the invention in aqueous or aqueous alcohol solution or other suitable solvents containing the required acid. Where a compound of the invention contains an acidic function, a base salt of said compound may be prepared by reacting said compound with a suitable base. The acid or base salt may separate directly or can be obtained by concentrating the solution e.g. by evaporation. The compounds of this invention may also exist in solvated or hydrated forms.

The invention also extends to a prodrug of the aforementioned compounds such as an ester or amide thereof. A prodrug is any compound that may be converted under physiological conditions or by solvolysis to any of the compounds of the invention or to a pharmaceutically acceptable salt of the compounds of the invention. A prodrug may be inactive when administered to a subject but is converted in vivo to an active compound of the invention.

The compounds of the invention may contain one or more asymmetric carbon atoms and may exist in racemic and optically active forms. The compounds of the invention may exist in trans or cis form. The first aspect of the invention covers all of these compounds.

The second aspect of the invention provides a process for the manufacture of a compound of formula (I) wherein R¹ is a group of formula (II) as defined in the first aspect of the invention comprising the condensation of an intermediate (III) with an intermediate (IV).

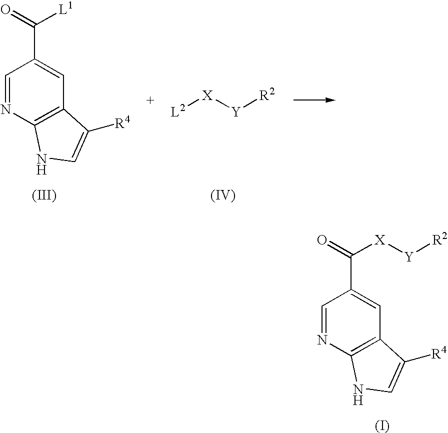

wherein R² and R⁴ are as defined in the first aspect of the invention; L¹ and L² are independently a leaving group wherein L¹ and L² together form a condensation product.

According to the process, a compound of the general formula (III), undergoes a condensation reaction with the compound of the general formula (IV), to form a compound of general formula I. In formulae (III) and (IV), L¹ and L² represent radicals that together form a condensation product, e.g. H and OH or H and Cl. Preferably L¹ is OH, OR⁵⁰, OM, Cl, Br or I wherein R⁵⁰ is C₁₋₆ alkyl, preferably methyl or ethyl and M is a metal, preferably Na, Li, K, Ca, Mg or Ba, and L² is preferably hydrogen or M. The condensation reaction occurs in a solution, preferably in a polar aprotic solvent such as e.g. dimethylformamide or dichloromethane. The condensation reaction may occur under the influence of coupling agents such as, for instance WSCHCl, DCC, benzotriazole-1-yl-oxy-tris-(dimethylamino)-phosphonium hexafluorophosphate (BOP), PyBrOP, etc., and in the presence of a tertiary amine (e.g. triethylamine) and 1-hydroxybenzotriazole (HOBT). Alternatively, the acid (III) may be first converted to an acid chloride by treatment with, for example, oxalyl chloride or thionyl chloride, and then without purification, reacted with, e.g. amines of formula (IV).

The third aspect of the invention provides a compound of formula (III)

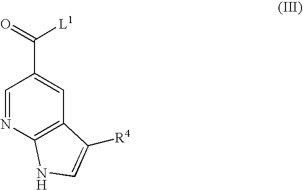

wherein R⁴ is as defined in the first aspect of the invention and L¹ is OH, OR⁵⁰, OM, Cl, Br, I;

R⁵⁰ is C₁₋₆ alkyl, preferably methyl or ethyl; and

M is metal, preferably Na, Li, K, Ca, Mg, Ba.

A compound of formula (III) may undergo one or more further reactions to provide a different compound of formula (III). For example, a compound may undergo a hydrolysis, reduction, oxidation, elimination, substitution and/or addition reaction.

The fourth aspect of the invention provides a process for the manufacture of a compound of formula (V) comprising removal of group R⁵¹ from an intermediate (VI)

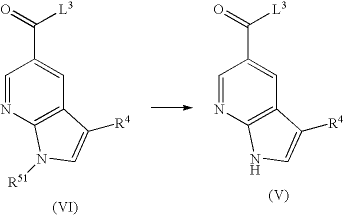

wherein L³ is R¹ or L¹,

wherein R¹ is as defined in the first aspect and

L¹ is as defined in the second or third aspect;

R⁴ is as defined in the first aspect, and

R⁵¹ is an amino protecting group. The pyrrole nitrogen can be protected using any protection known in the art. R⁵¹ can therefore include R⁵²SO₂, R⁵²C(O),

R⁵²₃Si, R⁵²OCH₂, (R⁵²)₂NSO₂, (R⁵²)₂NC(O), R⁵²OC(O), R⁵²(R⁵²O)CH, R⁵²CH₂CH₂, R⁵²CH₂, PhC(O)CH₂, CH₂═CH, ClCH₂CH₂, Ph₃C, Ph₂(4-pyridyl)C, Me₂N, HO—CH₂, R⁵²OCH₂, (R⁵²)₃SiOCH₂, (R⁵²O)₂CH, t-BuOC(O)CH₂, Me₂NCH₂, and tetrahydropyranylamine, wherein R⁵² is C₁₋₆ alkyl or C₆₋₁₂ aryl.

More preferably R⁵¹ is sulfonamide, most preferably benzenesulfonamide, (R⁵²)₂NSO₂, and (R⁵²)₂NC(O),

Removal of the protecting group can be afforded using conditions relevant to the protecting group used i.e. sulfonamide or amide protection can be removed by hydrolysis under basic conditions for example sodium hydroxide in water-ethanol, and silyl protection can be removed under acidic conditions for example TFA, HCl or using a source of fluoride, for example TBAF.

It will be appreciated that when L³ is R¹ the deprotection will afford directly the compound of formula (I).

It will be further appreciated than when L¹ is OR⁵⁰ the hydrolytic removal of R⁵¹ under basic conditions is accompanied by hydrolysis of the ester functionality to afford compound (III) where L¹ is OM, or OH after acidification of the reaction mixture.

The fifth aspect of the invention provides a compound of formula (VI)

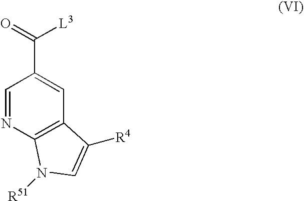

wherein R⁴ is as defined in the first aspect,

L³ is as defined in the fourth aspect; and

R⁵¹ is an amino protecting group as defined in the fourth aspect.

The sixth aspect of the invention provides a process for the manufacture of a compound of formula (VI) as defined in the fifth aspect of the invention comprising a a) reaction of a compound of formula (VII) with stannane R⁴-Sn(R⁵³)₃ in the presence of a palladium catalyst or b) reaction of a compound of formula (VII) with boronic acid or ester R⁴—B(OR⁵⁴)₂ in a presence of a suitable palladium catalyst or c) reaction of a compound of formula (VII) with silane R⁴—Si(R⁵⁵)₃ in the presence of a palladium catalyst;

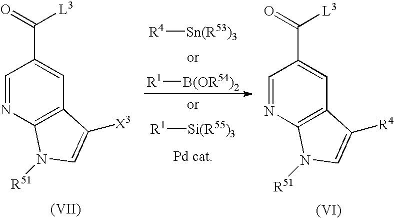

wherein R⁴ is as defined in the first aspect,

L³ is as defined in the fourth aspect,

R⁵¹ is an amino protecting group defined in the fourth aspect.

X³ is F, Cl, Br I or CF₃SO₃ preferably I or Br,

and wherein R⁵³ is independently C₁₋₆ alkyl;

R⁵⁴ is independently hydrogen or C₁₋₆ alkyl or wherein two R⁵⁴ groups together optionally form a five, six or seven membered ring with the boron and oxygen atoms, wherein the ring is optionally substituted with one or more C₁₋₆ alkyl group. Preferably, R⁵⁴ is hydrogen or both R⁵⁴ groups form the group —C(CH₃)₂—C(CH₃)₂—;

and R⁵⁵ is independently C₁₋₆ alkyl, F, OH.

Suitable catalysts for the purpose of this invention include (PPh₃)₂PdCl₂, (PPh₃)₄Pd, Pd(OAc)₂, [PdCl(η³-C₃H₅]₂, Pd₂(dba)₃, Pd(dba)₂ (dba=dibenzylidenacetone) and/or Pd/P(t-Bu)₃.

It will be appreciated that the reaction set out as option a) for the sixth aspect is a Stille reaction, which can be carried out according to Stille Angew. Chem., Int.ed, Engl. 1986, 25, 508; Mitchell Synthesis, 1992, 803, or Littke et al. J. Am. Chem. Soc. 2002, 124, 6343.

The reaction set out as option b) for the sixth aspect is a Suzuki reaction which can be carried out according to Suzuki Pure Appl. Chem. 1991, 63, 419 or Littke J. Am. Chem. Soc. 2000, 122, 4020

It will be appreciated that the reaction set out as option c) for the sixth aspect is a Hiyama reaction which can be carried out according to Hatanaka et al. J. Org. Chem. 1988, 53, 918, Hatanaka et al. Synlett, 1991, 845, Tamao et al. Tetrahedron Lett. 1989, 30, 6051 or Denmark et al. Org. Lett. 2000, 2, 565, ibid. 2491.

It will further be appreciated than when R⁵¹ is replaced with hydrogen the process of the sixth aspect yields a compound of formula (III) as defined in the fourth aspect of the invention.

The seventh aspect of the invention provides a compound of formula (VII)

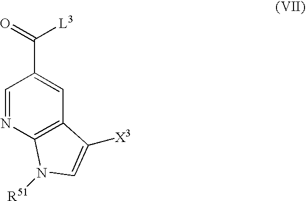

wherein L³ is as defined in the fourth aspect;

R⁵¹ is an amino protecting group as defined in the fourth aspect;

and X³ is as defined in the sixth aspect.

The eighth aspect of the invention provides a process for the manufacture of a compound of formula (VII) comprising protection of the pyrrole nitrogen of a compound of formula (VIII).

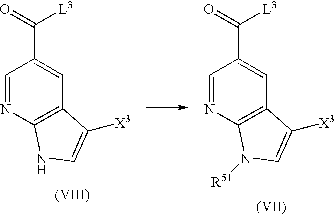

wherein L³ is as defined in the fourth aspect;

R⁵l is an amino protecting group defined in the fourth aspect, and

X³ is as defined in the sixth aspect

Conditions for the introduction of the protecting group R⁵¹ will depend upon the protecting group used. Compound (VII) can be produced by the initial formation of the relevant salt, for example by treatment with BuLi in THF or NaH in DMF, followed by reaction of the salt with an electrophile such as sulfonyl halide, or acid chloride. Alternatively a compound of formula (VII) can be produced by the direct reaction of compound (VIII) with an electrophile such as benzenesulfonyl halide, preferably benzenesulfonyl chloride. This reaction is preferably carried out in the presence of base (such as sodium hydroxide) and a phase transfer catalyst such as tetra-n-butylammonium bromide or tetra-n-butylammonium hydrogen sulphate.

The ninth aspect of the invention provides a compound of formula (VIII)

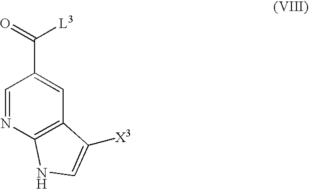

wherein L³ is as defined in the fourth aspect and

X³ is as defined in the sixth aspect.

The tenth aspect of the invention provides a process for the production of a compound of formula (VIII) by the introduction of an X³ group into a compound of formula (IX). Compound (VIII) can be produced from compound (IX) by halogenation under anhydrous conditions or by reaction with ICl under basic conditions (such as pyridine or i-Pr₂NEt in a chlorinated solvent such as CH₂Cl₂, CHCl₃, CCl₄) or NBS in an anhydrous solvent such as CH₂Cl₂, CHCl₃, CCl₄). Where X³ is iodine, it may preferably be introduced by direct action of I₂ on (IX) in the presence of a strong base such as sodium hydroxide or potassium hydroxide in anhydrous solvent such as dimethylformamide.

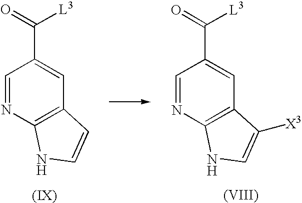

wherein L³ is as defined in the fourth aspect and X³ is as defined in the sixth aspect.

Preparation of compound of general formula (IX) wherein L³ is carbocyclyl or heterocyclyl has been disclosed in GB0305142.2.

The eleventh aspect of the invention provides compound of formula (IX)

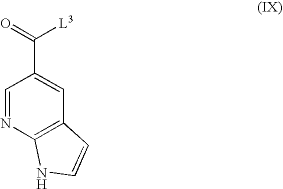

wherein L³ is as defined in the fourth aspect with the exception that L³ is not carbocyclyl or heterocyclyl;

in particular wherein L³ is a group L¹ as defined in the second aspect or a group R¹, wherein R¹ is a group of formula (II)

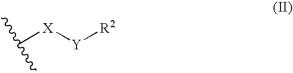

wherein X is NR³, O, S or (CR²²R²²)ₙ, Y is absent or is NR²³, O or (CR²³R²³)ₙ,

R² is optionally substituted C₁₋₁₂ alkyl, C₂₋₁₂ alkenyl, C₂₋₁₂ alkynyl, C₃₋₁₂ carbocyclyl or C₃₋₁₂ heterocyclyl as defined in the first aspect.

The twelfth aspect of the invention provides an alternative process for the production of a compound of formula (VII) by the introduction of the X³ group to a compound of formula (X).

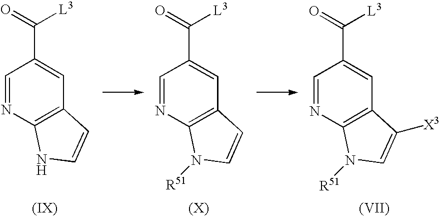

wherein R⁵¹ and L³ are as defined in the fourth aspect and X³ is as defined in the sixth aspect.

In particular where R⁵¹ is a silyl group, introduction of R⁵¹ occurs prior to the introduction of X³. Preparation of compound of general formula (X) wherein L³ is carbocyclyl or heterocyclyl has been disclosed in GB0305142.2

Thus, a skilled person will appreciate that the actual synthetic sequence to prepare compound (VII) will depend on the type of protecting group R⁵¹ used, i.e. the compound (VII) can be prepared by the process of the twelfth aspect or by the processes set out in the eighth and tenth aspects.

The thirteenth aspect of the invention provides a compound of formula (X)

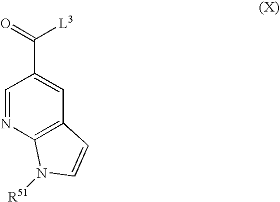

wherein L³ is as defined in the eleventh aspect and

wherein R⁵¹ is an amino protecting group as defined in the fourth aspect.

The fourteenth aspect of the invention provides a method for preparation of compound of formula (IX) by the acid-catalysed hydrolysis of nitrile (XI) in the presence of alcohol, preferably methanol or ethanol.

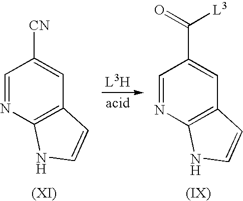

wherein L³ is OR⁵⁰;

and R⁵⁰ is as defined in the third aspect of the invention. Preferably R⁵⁰ is methyl or ethyl.

The acid-catalysed hydrolysis of compound (IX) is usually carried out by refluxing alcoholic solution of (IX) containing concentrated sulfuric acid. The product is isolated by neutralisation of the reaction mixture and extraction. It can be appreciated that thus prepared (XI) is an ester.

The fifteenth aspect of the invention provides a compound of formula (XI)

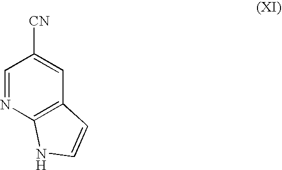

The sixteenth aspect of the invention provides a process for the manufacture of 1H-Pyrrolo[2,3-b]pyridine-5-carbonitrile (XI) comprising reaction of 5-bromo-1H-pyrrolo[2,3-b]pyridine with Zn(CN)₂ in the presence of a suitable palladium catalyst such as Pd(PPh₃)₄.

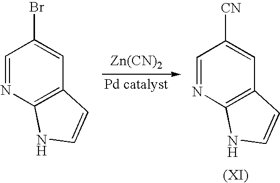

The present invention encompasses one or more compounds as defined in the third, fifth, seventh, ninth, eleventh, thirteenth and fifteenth of the invention as set out below;

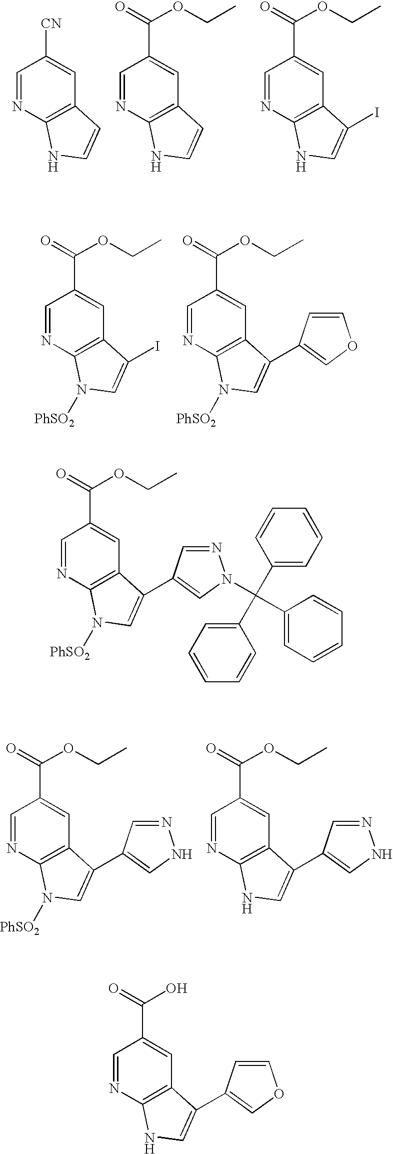

The present invention also encompasses a process for manufacturing a compound of the first aspect, the process comprising providing a starting material, which is commercially available or can be produced by a method known in the art, converting the starting material to form an intermediate compound of the third, fifth, seventh, ninth, eleventh, thirteenth and fifteenth aspects using a process as described above or a process known in the art (and optionally converting the intermediate compound so formed into another intermediate compound) and then converting the intermediate compound into a compound of the first aspect using a process as described above or a process known in the art (and optionally converting the compound of the first aspect so formed into another compound of the first aspect).

The seventeenth aspect of the invention provides a composition comprising a compound according to the first aspect of the invention in combination with a pharmaceutically acceptable carrier, diluent or excipient.

The composition may also comprise one or more additional active agent, such as an anti-inflammatory agent (for example a p38 inhibitor, glutamate receptor antagonist, or a calcium channel antagonist), AMPA receptor antagonist, a chemotherapeutic agent and/or an antiproliferative agent.

Suitable carriers and/or diluents are well known in the art and include pharmaceutical grade starch, mannitol, lactose, magnesium stearate, sodium saccharin, talcum, cellulose, glucose, sucrose, (or other sugar), magnesium carbonate, gelatin, oil, alcohol, detergents, emulsifiers or water (preferably sterile). The composition may be a mixed preparation of a composition or may be a combined preparation for simultaneous, separate or sequential use (including administration).

The composition according to the invention for use in the aforementioned indications may be administered by any convenient method, for example by oral (including by inhalation), parenteral, mucosal (e.g. buccal, sublingual, nasal), rectal or transdermal administration and the compositions adapted accordingly.

For oral administration, the composition can be formulated as liquids or solids, for example solutions, syrups, suspensions or emulsions, tablets, capsules and lozenges.

A liquid formulation will generally consist of a suspension or solution of the compound or physiologically acceptable salt in a suitable aqueous or non-aqueous liquid carrier(s) for example water, ethanol, glycerine, polyethylene glycol or oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and microcrystalline cellulose.

A composition in the form of a capsule can be prepared using routine encapsulation procedures. For example, powders, granules or pellets containing the active ingredient can be prepared using standard carriers and then filled into a hard gelatine capsule; alternatively, a dispersion or suspension can be prepared using any suitable pharmaceutical carrier(s), for example aqueous gums, celluloses, silicates or oils and the dispersion or suspension then filled into a soft gelatine capsule.

Compositions for oral administration may be designed to protect the active ingredient against degradation as it passes through the alimentary tract, for example by an outer coating of the formulation on a tablet or capsule.

Typical parenteral compositions consist of a solution or suspension of the compound or physiologically acceptable salt in a sterile aqueous or non-aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal or oral administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve, which is intended for disposal once the contents of the container have been exhausted. Where the dosage form comprises an aerosol dispenser, it will contain a pharmaceutically acceptable propellant. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal or vaginal administration are conveniently in the form of suppositories (containing a conventional suppository base such as cocoa butter), pessaries, vaginal tabs, foams or enemas.

Compositions suitable for transdermal administration include ointments, gels, patches and injections including powder injections.

Conveniently the composition is in unit dose form such as a tablet, capsule or ampoule.

The eighteenth aspect of the invention provides a process for the manufacture of a composition according to the seventeenth aspect of the invention. The manufacture can be carried out by standard techniques well known in the art and comprises combining a compound according to the first aspect of the invention and the pharmaceutically acceptable carrier or diluent and optionally one or more additional active agents. The composition may be in any form including a tablet, a liquid, a capsule, and a powder or in the form of a food product, e.g. a functional food. In the latter case the food product itself may act as the pharmaceutically acceptable carrier.

The nineteenth aspect of the present invention relates to a compound of the first aspect, or a composition of the seventeenth aspect, for use in medicine.

The compounds of the present invention are inhibitors of JNK, such as JNK1, JNK2, or JNK3. In particular, the compounds of the present invention are inhibitors of JNK3. Preferably, the compounds of the present invention inhibit JNK3 selectively (i.e. the compounds of the invention preferably show greater activity against JNK3 than JNK1 and 2). For the purpose of this invention, an inhibitor is any compound, which reduces or prevents the activity of the JNK enzyme.

The compounds are therefore useful for conditions for which inhibition of JNK activity is beneficial. Thus, preferably, this aspect provides a compound of the first aspect, or a composition of the seventeenth aspect of the present invention, for the prevention or treatment of a JNK-mediated disorder. The compounds of the first aspect of the invention may thus be used for the inhibition of JNK, more preferably for the inhibition of JNK3.

A “JNK-mediated disorder” is any disease or deleterious condition in which JNK plays a role. Examples include neurodegenerative disorder (including dementia), inflammatory disease, a disorder linked to apoptosis, particularly neuronal apoptosis, autoimmune disease, destructive bone disorder, proliferative disorder, cancer, infectious disease, allergy, ischemia reperfusion injury, heart attack, angiogenic disorder, organ hypoxia, vascular hyperplasia, cardiac hypertrophy, thrombin induced platelet aggregation and any condition associated with prostaglandin endoperoxidase synthase-2. The compounds of the present invention may be used for any of these JNK-mediated disorders.

The compounds of the present invention are particularly useful for the prevention or treatment of a neurodegenerative disorder. In particular, the neurodegenerative disorder results from apoptosis and/or inflammation. Examples of neurodegenerative disorders are: dementia; Alzheimer's disease; Parkinson's disease; Amyotrophic Lateral Sclerosis; Huntington's disease; senile chorea; Sydenham's chorea; hypoglycemia; head and spinal cord trauma including traumatic head injury; acute and chronic pain; epilepsy and seizures; olivopontocerebellar dementia; neuronal cell death; hypoxia-related neurodegeneration; acute hypoxia; glutamate toxicity including glutamate neurotoxicity; cerebral ischemia; dementia linked to meningitis and/or neurosis; cerebrovascular dementia; or dementia in an HIV-infected patient.

The neurodegenerative disorder may be a peripheral neuropathy, including mononeuropathy, multiple mononeuropathy or polyneuropathy. Examples of peripheral neuropathy may be found in diabetes mellitus, Lyme disease or uremia; peripheral neuropathy caused by a toxic agent; demyelinating disease such as acute or chronic inflammatory polyneuropathy, leukodystrophies, or Guillain-Barré syndrome; multiple mononeuropathy secondary to a collagen vascular disorder (e.g. polyarteritis nodosa, SLE, Sjögren's syndrome); multiple mononeuropathy secondary to sarcoidosis; multiple mononeuropathy secondary to a metabolic disease (e.g. diabetes or amyloidosis); or multiple mononeuropathy secondary to an infectious disease (e.g Lyme disease or HIV infection).

The compounds of the invention can also be used to prevent or treat disorders resulting from inflammation. These include, for example, inflammatory bowel disorder, bronchitis, asthma, acute pancreatitis, chronic pancreatitis, allergies of various types, and possibly Alzheimer's disease. Autoimmune diseases which may also be treated or prevented by the compounds of the present invention include rheumatoid arthritis, systemic lupus erythematosus, glumerulonephritis, scleroderma, chronic thyroiditis, Graves's disease, autoimmune gastritis, diabetes, autoimmune haemolytis anaemia, autoimmune neutropaenia, thrombocytopenia, atopic dermatitis, chronic active hepatitis, myasthenia gravis, multiple sclerosis, ulcerative colitis, Crohn's disease, psoriasis or graft vs host disease.

A compound of the present invention may be administered simultaneously, subsequently or sequentially with one or more other active agent, such as an anti-inflammatory agent e.g. p38 inhibitor, AMPA receptor antagonist, glutamate receptor antagonist, calcium channel antagonist, a chemotherapeutic agent or an antiproliferative agent. For example, for acute treatment, a p38 inhibitor may be administered to a patient prior to administering a compound of the present invention.

The compounds of the invention will normally be administered in a daily dosage regimen (for an adult patient) of, for example, an oral dose of between 1 mg and 2000 mg, preferably between 30 mg and 1000 mg, e.g. between 10 and 250 mg or an intravenous, subcutaneous, or intramuscular dose of between 0.1 mg and 100 mg, preferably between 0.1 mg and 50 mg, e.g. between 1 and 25 mg of the compound of the formula (I) or a physiologically acceptable salt thereof calculated as the free base, the compound being administered 1 to 4 times per day. Suitably the compounds will be administered for a period of continuous therapy, for example for a week or more.

The twentieth aspect of the invention relates to a method of treating or preventing a JNK-mediated disorder in an individual, which method comprises administering to said individual a compound of the first aspect or a composition of the seventeenth aspect. The active compound is preferably administered in a cumulative effective amount. The individual may be in need of the treatment or prevention. Any of the JNK-mediated disorders listed above in relation to the nineteenth aspect may be the subject of treatment or prevention according to the twentieth aspect. One or more other active agent may be administered to the individual simultaneously, subsequently or sequentially to administering the compound. The other active agent may be an anti-inflammatory agent such as a p38 inhibitor, glutamate receptor antagonist, AMPA receptor antagonist, calcium channel antagonist, a chemotherapeutic agent or an antiproliferative agent, but is preferably p38 inhibitor for acute treatment.

The twenty first aspect of the present invention provides the use of a compound of the first aspect in the manufacture of a medicament for the prevention or treatment of a JNK-mediated disorder. The medicament may be used for treatment or prevention of any of the JNK-mediated disorders listed above in relation to the nineteenth aspect. Again, the compound of the present invention may be administered simultaneously, subsequently or sequentially with one or more other active agent, preferably a p38 inhibitor for acute treatment.

In the twenty second aspect of the invention, there is provided an assay for determining the activity of the compounds of the present invention, comprising providing a system for assaying the activity and assaying the activity of the compound. Preferably the assay is for the JNK inhibiting activity of the compound, more preferably it is for the JNK3-specific inhibiting activity of the compounds. The compounds of the invention may be assayed in vitro, in vivo, in silico, or in a primary cell culture or a cell line. In vitro assays include assays that determine inhibition of either the kinase activity or ATPase activity of activated JNK. Alternatively, in vitro assays may quantitate the ability of a compound to bind JNK and may be measured either by radiolabelling the compound prior to binding, then isolating the inhibitor/JNK complex and determining the amount of the radiolabel bound or by running a competition experiment where new inhibitors are incubated with JNK bound to known radioligands. An example of an assay, which may be used, is Scintillation Proximity Assay (SPA), preferably using radiolabelled ATP. Another example is ELISA. Any type or isoform of JNK may be used in these assays.

In the twenty third aspect, there is provided a method of inhibiting the activity or function of a JNK, particularly JNK3, which method comprises exposing a JNK to a compound or a composition of the first or seventeenth aspect of the present invention. The method may be performed in a research model, in vitro, in silico, or in vivo such as in an animal model. A suitable animal model may be a kainic acid model in rat or mice, traumatic brain injury model in rat, or MPTP in nice.

All features of each of the aspects apply to all other aspects mutatis mutandis.

The invention will now be illustrated by the following non-limiting examples.

### EXAMPLES

### Synthesis of Example Inhibitor 8

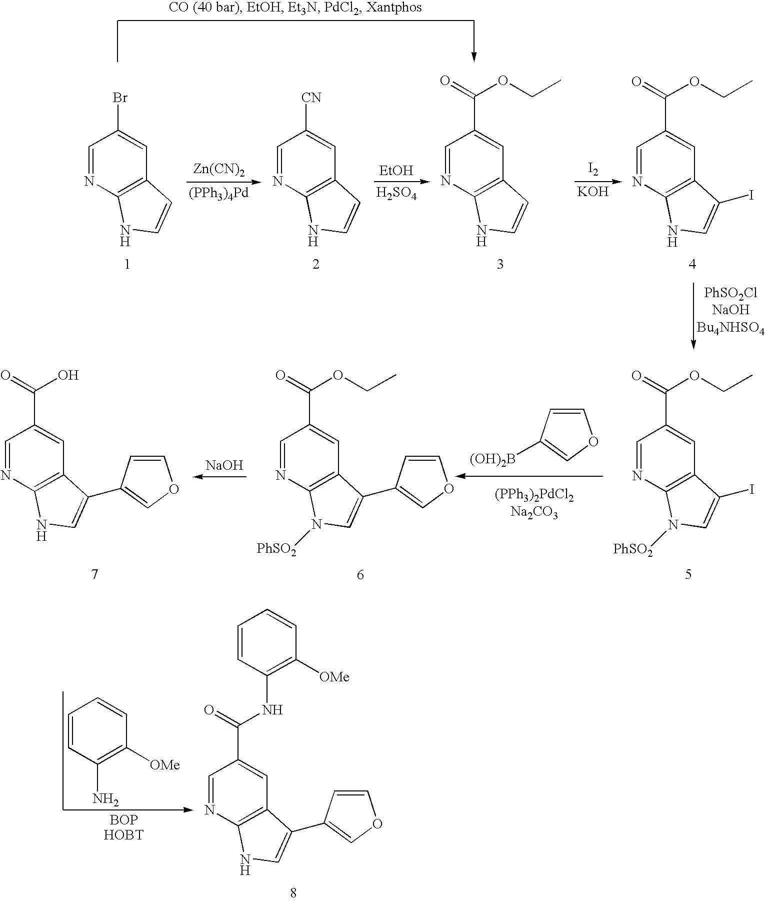

### 1H-Pyrrolo[2,3-b]pyridine-5-carbonitrile (2)

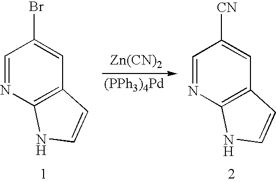

A mixture of bromide 1 (10.0 g, 50.8 mmol), ZnCl₂ (3.58 g, 30.5 mmol), and Pd(PPh₃)₄ (3.52 g, 3.05 mmol) in DMF (110 mL) was heated at 80° C. overnight. The solvent was evaporated and the residue separated by silicagel chromatography (100 g column) using hexane:ethyl acetate as eluent (gradient elution). The resulting solid was partitioned between water (200 mL)/CH₂Cl₂ (100 mL) and the aqueous phase extracted with more CH₂Cl₂ (4×100 mL). The combined organic extracts were dried (MgSO₄) and concentrated to give product 2 as a white solid (5.48 g, 75%), which was used for subsequent reactions without further purification.

### 1H-Pyrrolo[2,3-b]pyridine-5-carboxylic acid ethyl ester (3)

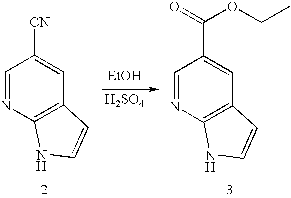

A solution of 2 (197.6 mg, 1.38 mmol) in a mixture of EtOH (4.2 mL) and concentrated H₂SO₄ (2.0 mL) was refluxed overnight. The reaction mixture was cooled and poured slowly onto a mixture of NaHCO₃ (8.2 g, solid), ice (50 g) and ethyl acetate (20 mL). The organic layer was separated. The aqueous layer was extracted with ethyl acetate (3×20 mL). Combined organic solutions were dried (MgSO₄), concentrated and dried in vacuum to afford ethyl ester 3 (262.5 mg, 100%) as white solid; 1H NMR (400 MHz, CDCl₃) δ 1.44 (t, J=7.2 Hz, 3H), 4.43 (q, J=7.2 Hz, 2H), 6.62 (dd, J=3.6, 2.0 Hz, 1H), 7.39 (dd, J=3.6, 2.4 Hz, 1H), 8.63 (dd, J=2.0, 0.8 Hz, 1H), 9.01 (d, J=2.0 Hz, 1H), 9.21 (bs, 1H).

### 1H-Pyrrolo[2,3-b]pyridine-5-carboxylic acid ethyl ester (3)—an alternative method

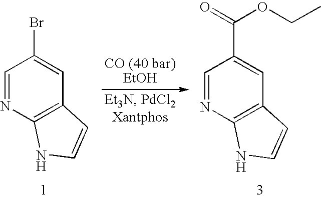

An autoclave charged with a mixture of 1 (9.85 g, 50.0 mmol), PdCl₂ (44 mg, 0.25 mmol), Xantphos (145 mg, 0.25 mmol), Et₃N (9.0 mL, 64.6 mmol) in EtOH (55 mL) was purged with CO. Then, CO was introduced to the pressure of 40 bar and the temperature of the reaction mixture was raised to 120° C. The mixture was stirred at 120° C. overnight. The mixture was cooled to room temperature and CO was released. ¹H NMR of an aliquot showed conversion of 80%. New portion of PdCl₂ (44 mg, 0.25 mmol) and Xantphos (145 mg, 0.25 mmol) was added. The autoclave was pressurized with CO again and raised to 120° C. After additional 3 days stirring at 120° C. the reaction was completed (¹H NMR). The reaction mixture was concentrated and separated between AcOEt—saturated aqueous NaHCO₃. The aqueous layer was extracted with AcOEt (5×100 mL). Combined organic solutions were dried (MgSO₄), and concentrated to afford 3 (7.55 g, 79%) as tan solid indistinguishable (¹H NMR) from the sample prepared via nitrile 2.

### 3-Iodo-1H-pyrrolo[2,3-b]pyridine-5-carboxylic acid ethyl ester (4)

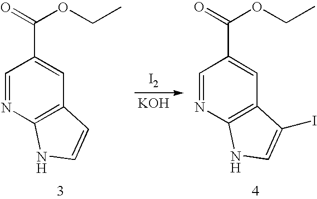

To a solution of 3 (100 mg, 0.526 mmol) in DMF (1.3 mL) was added 12 (162.56 mg, 0.64 mmol) followed by KOH (43.0 mg, 0.77 mmol). The reaction mixture was stirred for 35 min, and treated with a mixture of 0.1 M phosphate buffer (2.0 mL): saturated aqueous Na₂S₂O₃ (0.5 mL). The suspension was stirred at r.t. for 1 5 min. The solid precipitate was filtered off, washed with water (2.0 mL), and dried under high vacuum to give 4 as white solid (123.0 mg, 74%); ¹H NMR (400 MHz, DMSO-d₆) δ 1.34 (t, J=7.1 Hz, 3H), 4.35 (q, J=7.1 Hz, 2H), 7.87 (s, 1H), 8.15 (d, J=2.0 Hz, 1H), 8.80 (d, J=2.0 Hz, 1H), 12.50 (bs, 1H).

### 1-Benzenesulfonyl-3-iodo-1H-pyrrolo[2,3-b]pyridine-5-carboxylic acid ethyl ester (5)

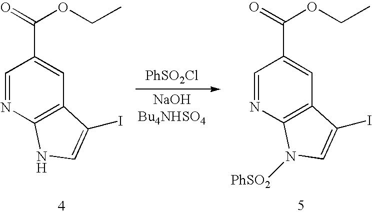

To a suspension of 4 (112.5 mg, 0.356 mmol) in CH₂Cl₂ (2.2 mL) was added benzenesulfonyl chloride (69.6 μL, 0.55 mmol), tetra-n-butylammonium hydrogen sulfate (14.9 mg, 0.044 mmol) and 50% aqueous NaOH (29 μL), and the reaction mixture was stirred for 1 h. The organic layer was separated and the residue was extracted with CH₂Cl₂ (2×3 mL). The combined organic solutions were dried (MgSO₄) and concentrated to give oil, which was triturated with methanol (1.0 mL) to give white solid. The solid was filtered off, washed with methanol (2×1 mL) and dried overnight in vacuum to give product 5 as a white solid (139.2 mg, 64%); ¹H NMR (400 MHz, CDCl₃) δ 1.42 (t, J=7.1 Hz, 3H), 4.43 (q, J=7.1 Hz, 2H), 7.49-7.53 (m, 2H), 7.59-7.64 (m, 1H), 7.94 (s, 1H), 8.21-8.25 (m, 2H), 8.31 (d, J=2.0 Hz, 1H), 9.08 (d, J=2.0 Hz, 1H).

### 1-Benzenesulfonyl-3-furan-3-yl-1H-pyrrolo[2,3-b]pyridine-5-carboxylic acid ethyl ester (6)

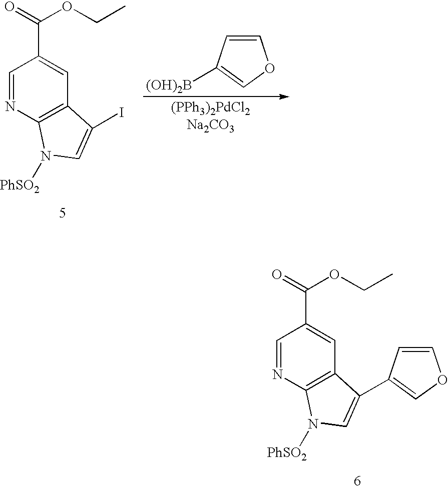

A mixture of 5 (100 mg, 0.219 mmol), EtOH (1.3 mL), toluene (1.3 mL), furan-3-boronic acid (37.4 mg, 0.33 mmol), 1M aq. Na₂CO₃ (0.55 mL, 0.55 mmol), LiCl (28 mg, 0.66 mmol) and PdCl₂(PPh₃)₂ (12.9 mg, 18.4 [mol) was refluxed for 17 min. The organic layer was separated, brine was added, and the aqueous layer was extracted with AcOEt. The combined organic solutions were concentrated and separated by means of silicagel chromatography using hexane:CH₂Cl₂ as eluent (in gradient up to 15% AcOEt) to give 6 as a tan solid (66.8 mg, 77%); ¹H NMR (400 MHz, CDCl₃) δ 1.42 (t, J=7.1 Hz, 3H), 4.43 (q, J=7.1 Hz, 2H), 6.71 (dd, J=1.8, 0.9 Hz, 1H), 7.48-7.54 (m, 2H), 7.56 (t, J=1.7 Hz, 1H), 7.58-7.63 (m, 1H), 7.85 (t, J=0.9 Hz, 1H), 7.89 (s, 1H), 8.22-8.27 (m, 2H), 8.60 (d, J=2.0 Hz, 1H), 9.12 (d, J=2.0 Hz, 1H).

### 3-Furan-3-yl-1H-pyrrolo[2,3-b]pyridine-5-carboxylic acid (7)

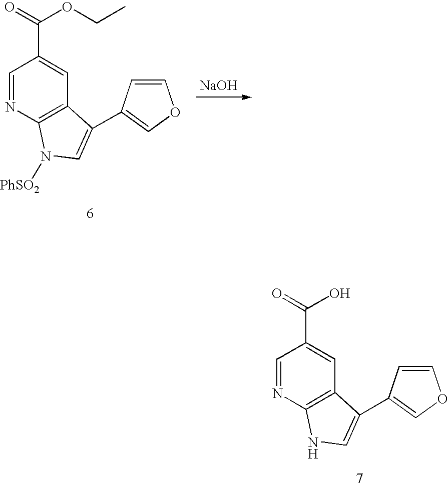

To a suspension of 6 (65.5 mg, 0.165 mmol) in EtOH (1.0 mL), was added 10% aqueous NaOH (0.5 mL, about 1.25 mmol), and the reaction mixture was refluxed for 0.5 h. The mixture was cooled to r.t. and EtOH was evaporated in vacuum. The residual solution was treated with glacial acetic acid (75 μL, 1.25 mmol). The suspension which formed was stirred at r.t. for 30 min. The solid was filtered off, washed with water, and dried under high vacuum to afford 7 as a tan powder (34.1 mg, 91 %); ¹H NMR (400 MHz, DMSO-d₆) δ 6.94 (d, J=1.7 Hz, 1H), 7.74 (t, J=1.5 Hz, 1H), 7.88 (d, J=2.2 Hz, 1H), 8.17 (s, 1H) (d, J=1.8 Hz, 1H), 8.81 (d, J=1.8 Hz, 1H), 12.16 (bs, 1H).

### 3-Furan-3-yl-1H-pyrrolo[2,3-b]pyridine-5-carboxylic acid (2-methoxy-phenyl)-amide (8)

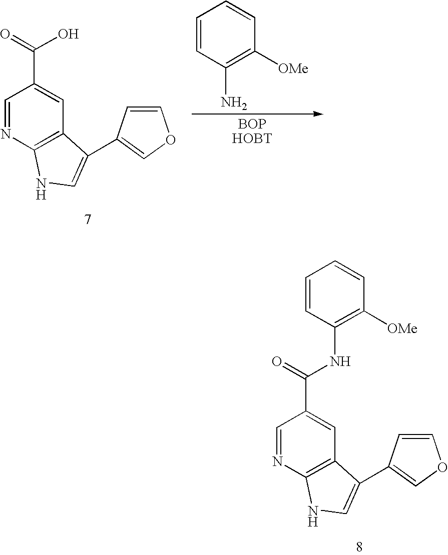

A mixture of 2-methoxy-phenylamine (21.55 mg, 0.175 mmol), carboxylic acid 7 (20 mg, 88 μmol), BOP (50.42 mg, 0.114 mmol), HOBT (17.8 mg, 0.132 mmol) and i-Pr₂NEt (30.7 μL, 0.176 mmol) in dry DMF (0.6 mL) was stirred at r.t. for 2 h. Separation of the crude reaction mixture by LCMS (column LUNA 10μ C18(2) 00G-4253-VO 250×50 mm) using water—acetonitrile (0.1% AcOH) as eluent (in gradient; flow 80 mL/min) afforded amide 8 (13.11 mg, 45%) as a white solid. ¹H NMR (400 MHz, CDCl₃:5% CD₃OD) δ 3.93 (s, 3H), 6.71 (dd, J=1.8, 0.8 Hz, 1H), 6.94 (dd, J=8.0, 1.5 Hz, 1H), 7.03 (ddd, J=7.9, 7.7, 1.5 Hz, 1H), 7.10 (ddd, J=8.0, 7.7, 1.7 Hz, 1H), 7.50 (s, 1H), 7.53 (t, J=1.7 Hz, 1H), 7.84 (t, J=1.1 Hz, 1H), 8.48 (dd, J=7.9, 1.7 Hz, 1H), 8.61 (d, J=2.0 Hz, 1H), 8.81 (d, J=2.0 Hz, 1H); LCMS m/e 334 (M+H), 375 (M+MeCN+H).

### Synthesis of Example Inhibitor 16

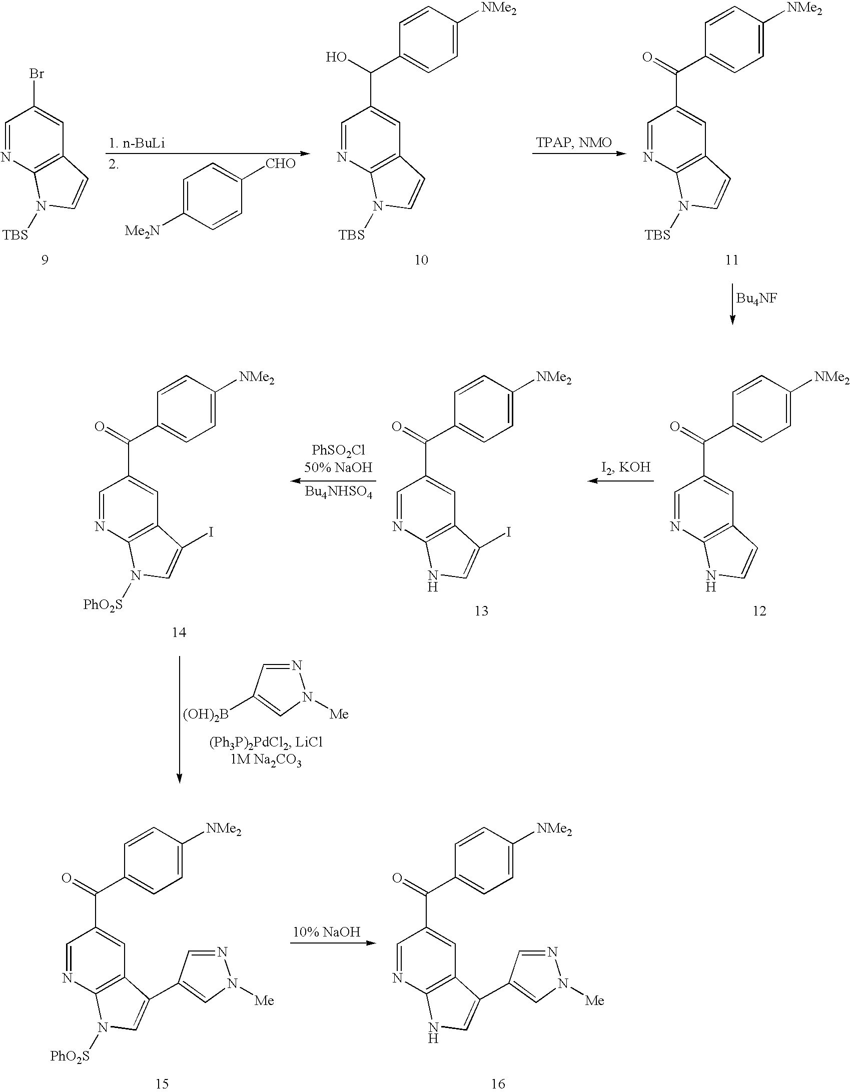

### [1-(tert-Butyl-dimethyl-silanyl)-1H-pyrrolo[2,3-b]pyridin-5-yl]-(4-dimethylamino-phenyl)-methanol (10)

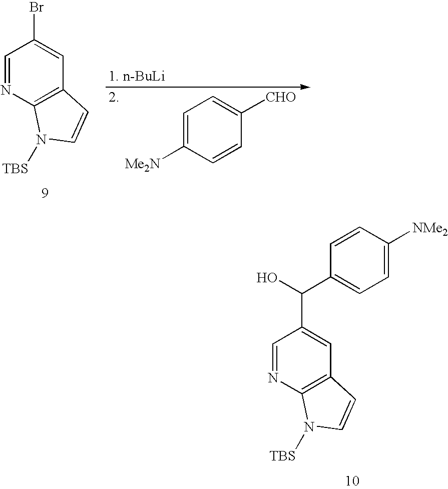

To a −78° C. stirred solution of the bromo-azaindole 9 (2 g, 6.4 mmol; preparation disclosed in WO2004/078757) in THF (10 mL) was added a 2.5 M solution of n-butyllithium in hexanes (5.4 mL, 13.5 mmol) dropwise. The resulting yellow solution was stirred for 0.6 h at −78° C. and then 4-dimethylamino-benzaldehyde (1.25 g, 8.4 mmol) in THF (10 mL) was added slowly. The mixture was allowed to warm to room temperature and after a further 20 h diluted with EtOAc and saturated brine and partitioned. The aqueous layer was extracted with EtOAc (2×). The combined organic solutions were dried (MgSO₄) and concentrated in vacuo. The crude residue was purified by silica gel chromatography employing Et₃N-impregnated silica and AcOEt:hexane as eluent (gradient) to afford a 2.5:1 mixture of the alcohol 10 and 4-dimethylamino-benzaldehyde (1.63 g, 47%) as a yellow oil. The partially purified alcohol 10 was used directly for the next step without any further purification.

### [1-(tert-Butyl-dimethyl-silanyl)-1H-pyrrolo[2,3-b]pyridin-5-yl]-(4-dimethylamino-phenyl)-methanone (11)

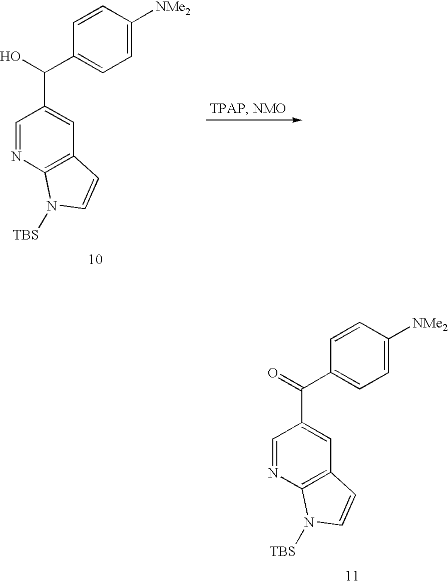

To a stirred solution of a 2.5:1 mixture of alcohol 11 and 4-dimethylamino-benzaldehyde (1.23 g, 2.3 mmol), 4-methylmorpholine N-oxide (0.41 g, 3.5 mmol), 4A powdered molecular sieves (1.2 g) in CH₂Cl₂ (12 mL) was added TPAP (82 mg, 0.23 mmol) in one portion. After 3 h the mixture was filtered through a pad of silica and the silica pad washed with CH₂Cl₂. The combined organic solutions were concentrated to afford 11 as black oil that was used directly in the next step without any purification. ¹H NMR (400 MHz; CDCl₃) δ 0.66 (s, 6H), 0.95 (s, 9H), 3.09 (s, 6H), 6.62 (d, J=3.5 Hz, 1H), 6.70 (d, J=9.2 Hz, 2H), 7.31 (d, J=3.5 Hz, 1H), 7.83 (d, J=9.1 Hz, 2H), 8.27 (d, J=2.1 Hz 1H) and 8.71 (d, J=2.1 Hz, 1H).

### (4-Dimethylamino-phenyl)-(1H-pyrrolo[2,3-b]pyridin-5-yl)-methanone (12)

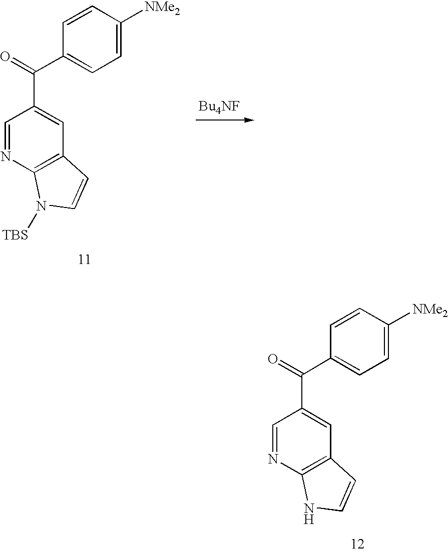

To a stirred solution of the crude ketone 11 (assumed 880 mg, 2.3 mmol) in THF (13 mL) was added 1M TBAF in THF (3.5 mL, 3.5 mmol) dropwise. After 3 h the mixture was concentrated to dryness and diluted with AcOEt and saturated brine, and partitioned. The aqueous layer was extracted with AcOEt (3×) and the combined organic extracts dried (MgSO₄), filtered and concentrated in vacuo. The residue was purified by silica gel chromatography using hexane:AcOEt (gradient elution) to afford ketone 12 (298 mg, 48% over 2 steps). ¹H NMR (400 MHz; CDCl₃) δ 3.10 (s, 6H), 6.62 (d, J=3.4 Hz, 1H), 6.72 (d, J=9.0 Hz, 2H), 7.42 (d, J=3.3 Hz, 1H), 7.84 (d, J=9.0 Hz, 2H), 8.38 (d, J=1.9 Hz, 1H), 8.78 (d, J=1.9 Hz, 1H) and 10.06 (brs, 1H).

### (4-Dimethylamino-phenyl)-(3-iodo-1H-pyrrolo[2,3-b]pyridin-5-yl)-methanone (13)

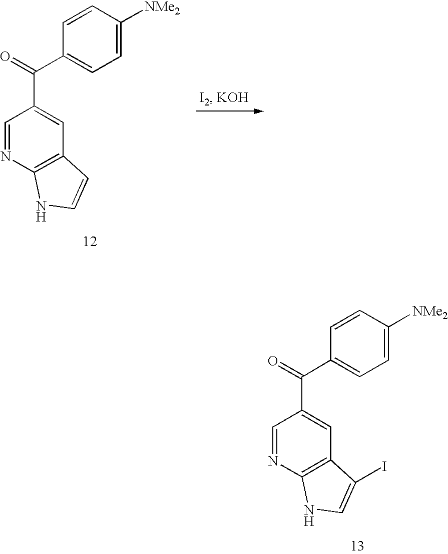

To a stirred solution of the ketone 12 (295 mg, 1.1 mmol) in DMF (7.5 mL) was added potassium hydroxide pellets (235 mg, 4.2 mmol). After 0.3 h, iodine (254 mg, 1.0 mmol) was added in one portion. Following a further 5 h the mixture was diluted with AcOEt and saturated sodium thiosulfate, and stirred vigoursly for 5 minutes. The mixture was partitioned and the aqueous layer extracted with AcOEt (3×). The combined organic extracts were dried (MgSO₄), filtered and concentrated in vacuo to afford iodide 13 as brown solid that was used directly in the next step without any purification.

### (1-Benzenesulfonyl-3-iodo-1H-pyrrolo[2,3-b]pyridin-5-yl)-(4-dimethylamino-phenyl)-methanone (14)

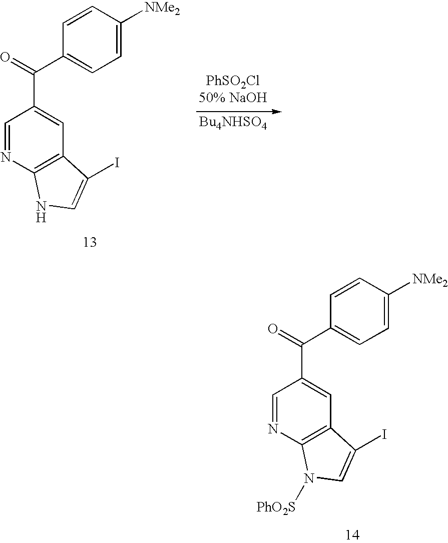

To a stirred solution of the iodide 13 (assumed 435 mg, 1.1 mmol) in CH₂Cl₂ (13 mL) was added benzenesulfonyl-chloride (304 mg, 1.7 mmol), 50% NaOH (1 mL) and n-tetra-n-butyl ammonium sulfate (57 mg, 0.17 mmol). After 4.5 h the mixture was diluted with AcOEt and saturated sodium hydrogen carbonate solution and partitioned. The aqueous layer was extracted with EtOAc (3×) and the combined organic extracts were dried (MgSO₄), filtered and concentrated in vacuo. The residue was treated with MeOH and stirred vigoursly for 0.5 h and then vacuum filtered to afford the iodide 14 (410 mg, 69% over 2 steps) as a tan solid. ¹H NMR (400 MHz; CDCl₃) □ 3.10 (s, 6H), 6.69 (J=9.1 Hz, 2H), 7.51 (m, 2H), 7.62 (tt, J=1.2, 1.9 and 7.5 Hz, 1H), 7.77 (d, J=9.1 Hz, 2H), 7.95 (s, 1H), 8.04 (d, J=1.9 Hz, 1H), 8.23 (m, 2H), 8.79 (d, J=1.9 Hz, 1H).

### [1-Benzenesulfonyl-3-(1-methyl-1H-pyrazol-4-yl)-1H-pyrrolo[2,3-b]pyridin-5-yl]-(4-dimethylamino-phenyl)-methanone (15)

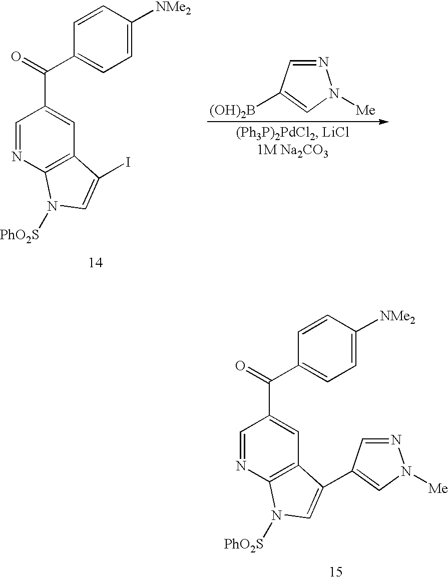

A mixture of iodide 14 (100 mg, 0.19 mmol), 1-methyl-1H-pyrazole-4-boronic acid (36 mg, 0.28 mmol), lithium chloride (24 mg, 0.56 mmol), dichlorobis(triphenylphosphine)-palladium (II) (7 mg, 0.01 mmol), 1M sodium carbonate (0.47 mL, 0.47 mmol) in toluene (2 mL) and ethanol (2 mL) was heated at 105° C. for 6 h. Then, the reaction mixture was cooled to room temperature and partitioned between AcOEt and saturated brine. The aqueous layer was extracted with AcOEt (3×). The combined organic extracts were dried (MgSO₄), filtered and concentrated. The residue was purified by preparative TLC using hexane:AcOEt=1:1 (v/v) as eluent to afford ketone 15 (51 mg, 56%). ¹H NMR (400 MHz; CDCl₃) δ 3.10 (s, 6H), 3.98 (s, 3H), 6.67 (d, J=9.1 Hz, 2H), 7.51 (m, 2H), 7.59 (m, 1H), 7.69 (s, 1H), 7.79 (m, 3H), 7.86 (s, 1H), 8.23 (m, 2H), 8.36 (d, J=1.9 Hz, 1H) and 8.81 (d, J=1.9 Hz, 1H).

### (4-Dimethylamino-phenyl)-[3-(1-methyl-1H-pyrazol-4-yl)-1H-pyrrolo[2,3-b]pyridin-5-yl]-methanone (16)

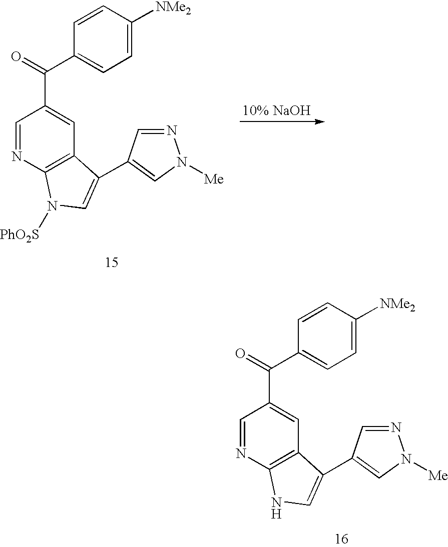

To a solution of the ketone 15 (51 mg, 0.11 mL) in ethanol (10 mL) was added 10% sodium hydroxide (1 mL) and the mixture stirred at 90° C. for 3 h. The mixture was concentrated to remove ethanol and partitioned between AcOEt and saturated brine. The aqueous layer was extracted with AcOEt (3×). The combined organic extracts were dried (MgSO₄), filtered and concentrated. The residual orange oil was purified by preparative TLC with AcOEt as eluent to afford inhibitor 16 (14 mg, 39%). ¹H NMR (400 MHz; CDCl₃) δ 3.10 (s, 6H), 3.98 (s, 3H), 6.72 (d, J=9.1 Hz, 2H), 7.50 (d, J=2.0 Hz, 1H), 7.68 (s, 1H), (s, 1H), 7.86 (d, J=9.1 Hz, 2H), 8.51 (d, J=1.9 Hz, 1H), 8.78 (d, J=1.9 Hz, 1H) and 10.28 (brs, 1H).

Biological Activity

JNK1, JNK2, JNK3—SPA Assay
1. Compound is dissolved in DMSO to a convenient concentration and this is diluted in 10% DMSO to a five times concentrate of the desired starting concentration (frequently 1:100).
2. 10 μl of 500 mM EDTA is added to alternative wells of the Opti-plate row, which will receive kinase reaction plus DMSO. This creates the negative control.
3. For the JNK2 and JNK3 assay, compounds are prepared in six 2-fold dilutions with water and each concentration is tested in duplicate. For the JNK1 assay compounds are prepared in four 5-fold dilutions with water which are tested in triplicate. Controls are treated identically.
4. 20 μl per well of each compound concentration is transferred to an Opti-plate, in duplicate.
5. 30 μl (JNK2/3 SPA) or 50 μl (JNK1 SPA) of substrate solution (25 mM HEPES pH 7.5, 10 mM magnesium acetate with 3.33 μM ATP (JNK2/3) or 2 μM ATP (JNK1), approximately 7.5 kBq [γ-³³P] ATP, GST-c-Jun, in water) is added to each well.
6. 50 μl (JNK2/3 SPA) or 30 μl (JNK1 SPA) of kinase solution (JNK in 25 mM HEPES pH 7.5, 10 mM Mg Acetate) is added to each well.

| Kinase | Kinase per well (μg) | GST-c-Jun per well (μg) |
|---|---|---|
| JNK1 | 0.25 | 1 |
| JNK2 | 0.2 | 1.2 |
| JNK3 | 0.16 | 1.2 |

7. The plate is incubated for 30 minutes at room temperature.
8. 100 μl of bead/stop solution is added to each well (5 mg/ml glutathione-PVT-SPA beads, 40 mM ATP in PBS).
9. Plates are sealed and incubated for 30 minutes at room temperature, centrifuged for 10 minutes at 2500 g and counted.
10. The IC₅₀ values are calculated as the concentration of the compound being tested at which the phosphorylation of c-Jun is decreased to 50% of the control value. Example IC₅₀ values for the compounds of this invention are given in Table 1.

p38 ELISA

Active p38 kinase (100 ng; Upstate) was added to 2 μg GST-ATF2 substrate (NEB) in 250 mM Hepes pH 7.5/100 mM MgAc/50 82 M ATP (final) in the presence or absence of compounds in 50 μl. The mixture was incubated at 30° C. for 1 hour, and then diluted with 200 μl PBS-Tween (0.05%). From this, duplicate volumes of 100 μl were added to a Reacti-Bind glutathione coated plate (Pierce) and incubated for 1 hour. After washing 3 times with PBS-Tween (0.05 %), rabbit anti-phospho-ATF2 (Thr71) antibody (NEB) was added at 1:500, and incubated for another hour at room temperature. After 3 additional washes with PBS-Tween (0.05 %), 100 μl of anti-rabbit IgG alkahne phosphatase-conjugated secondary antibody (Sigma) was added at 1:1000, the reaction was incubated for a further hour, washed 3 times, and then phosphatase substrate (Sigma) was added (100 μl per well; 3 tablets in 5 ml water). After incubation in the dark at 37° C. for 1 hour, the reaction mixture was transferred to a clear 96 well plate, and the absorbance at 405 nm was read. The IC₅₀ values are calculated as the concentration of the compound being tested at which the phosphorylation of ATF2 is decreased to 50% of the control value. Example IC₅₀ values for the compounds of this invention are given in Table 1 (last column).

Table 1. IC₅₀ values for selected compounds against JNK1, JNK2, JNK3, and p38 MAP kinase

| | JNK3 |
|---|---|
| Compound | IC₅₀ (nm) |
| 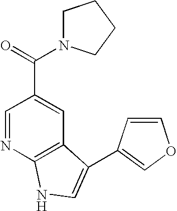 | <500 |
| 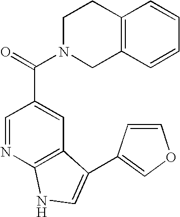 | <500 |
| 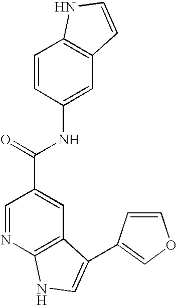 | <1000 |
| 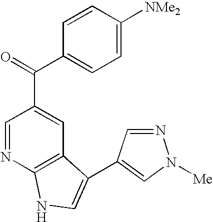 | <500 |
| 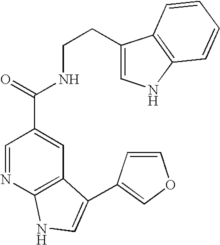 | <500 |

1. A compound of formula (I): 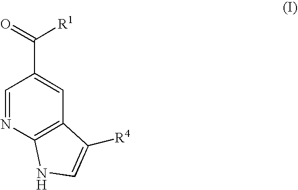
and the pharmaceutically acceptable salts, and other pharmaceutically acceptable biohydrolyzable derivatives thereof;
wherein R¹ is an optionally substituted C₃₋₁₂ carbocyclyl or C₃₋₁₂ heterocyclyl group or group of formula (II) 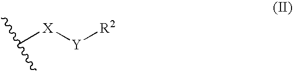
wherein X is NR³, O, S or (CR²²R²²)ₙ, Y is absent or is NR²³, O, or (CR²³R²³)ₙ, R² is optionally substituted C₁₋₁₂ alkyl, C₂₋₁₂ alkenyl, C₂₋₁₂ alkynyl, C₃₋₁₂ carbocyclyl or C₃₋₁₂ heterocyclyl, and R⁴ is an optionally substituted five or six membered heterocyclyl group or an optionally substituted six membered carbocyclyl group.

2. A compound as claimed in claim 1 wherein the optionally substituted carbocyclyl or heterocyclyl group of R¹ is optionally fused to a partially saturated, unsaturated or fully saturated five to seven membered ring containing zero to three heteroatoms, and each substitutable carbon atom in R¹, including the optional fused ring, is optionally and independently substituted by one or more of halogen, C₁₋₁₂ alkyl, C₂₋₁₂ alkenyl, C₂₋₁₂ alkynyl, haloC₁₋₁₂alkyl, C₃₋₁₂ carbocyclyl, C₃₋₁₂ heterocyclyl, (CH₂)ₙOR⁵, (CH₂)ₙNR⁵₂(CH₂)ₙSR⁵, OR⁵, SR⁵, NO₂, CN, NR⁵₂, NR⁵COR⁵, NR⁵CONR⁵₂, NR⁵COR⁵, NR⁵CO₂R⁵, COR⁵, CORN⁵₂, S(O)₂R⁵, SONR⁵₂, S(O)R⁵, SO₂NR⁵₂, or NR⁵S(O)₂R⁵ wherein the C₁₋₁₂ alkyl group optionally contains one or more insertions selected from —O—, —N(R⁵)— —S—, —S(O)— and —S(O₂)—; and each saturated carbon in the optional fused ring is further optionally and independently substituted by ═O, ═S, NNR⁶₂, ═N—OR⁶, ═NNR⁶COR⁶, ═NNR⁶CO₂R⁶, ═NNSO₂R⁶, or ═NR⁶; and each substitutable nitrogen atom in R¹ is optionally substituted by R⁷, COR⁷ SO₂R⁷ or CO₂R⁷;
wherein n is 1 to 6, preferably n is 1, 2 or 3;
wherein R⁵ is hydrogen, C₁₋₁₂ alkyl, C₃₋₁₂ carbocyclyl or C₃₋₁₂ heterocyclyl, optionally substituted by one or more of C₁₋₆ alkyl, C₃₋₁₂ carbocyclyl, C₃₋₁₂ heterocyclyl, halogen, C₁₋₆ haloalkyl, OR⁸, SR⁸, NO₂, CN, NR⁸R⁸, NR⁸COR⁸, NR⁸CONR⁸R⁸, NR⁸COR⁸, NR⁸CO₂R⁸, COR⁸, CONR⁸₂, S(O)₂R⁸, SONR⁸₂, S(O)R⁸, SO₂NR⁸R⁸, NR⁸ S(O)₂R⁸, wherein the C₁₋₁₂ alkyl group optionally incorporates one or two insertions selected from the group consisting of —O—, —N(R⁸)—, —S(O)— and —S(O₂)—, wherein each R⁸ may be the same or different and is as defined below;
wherein two R⁵ in NR⁵₂ may optionally form a partially saturated, unsaturated or fully saturated three to seven membered ring containing one to three heteroatoms, optionally and independently substituted by one or more of C₁₋₆ alkyl, halogen, C₁₋₆ haloalkyl, OR⁸, SR⁸, NO₂, CN, NR⁸R⁸, NR⁸COR⁸, NR⁸CONR⁸R⁸, NR⁸COR⁸, NR⁸CO₂R⁸, CO₂R⁸, COR⁸, CONR⁸₂, S(O)₂R⁸, SONR⁸₂, S(O)R⁸, SO₂NR⁸R⁸, NR⁸S(O)₂R⁸,
wherein the C₁₋₆ alkyl group optionally incorporates one or two insertions selected from the group consisting of —O—, —N(R⁸)—, —S(O)— and —S(O₂)—, wherein each R⁸ may be the same or different and is as defined below;
wherein R⁶ is hydrogen, C₁₋₁₂ alkyl, C₃₋₁₂ carbocyclyl or C₃₋₁₂ heterocyclyl, optionally substituted by one or more of C₁₋₆ alkyl, halogen, C₁₋₆ haloalkyl, OR⁸, SR⁸, NO₂, CN, NR⁸R⁸, NR⁸COR⁸, NR⁸CONR⁸R⁸, NR⁸COR⁸, NR⁸CO₂R⁸, CO₂R⁸, COR⁸, CONR⁸₂, S(O)₂R⁸, S(O)R⁸, SO₂NR⁸R⁸, NR⁸S(O)₂R⁸, wherein the C₁₋₁₂ alkyl group optionally incorporates one or two insertions selected from the group consisting of —O—, —N(R 8)—, —S(O)— and —S(O₂)—, wherein each R⁸ may be the same or different and is as defined below;
wherein R⁷ is hydrogen, C₆₋₁₂ aryl, C₁₋₆ alkyl or C₁₋₆ haloalkyl;
wherein R⁸ is hydrogen, C₁₋₆ alkyl, or C₁₋₆ haloalkyl.

3. A compound as claimed in claim 1 wherein Y is absent or is NR²³, O, (CR²³R²³)ₙ,
wherein each R²³ is H, C₁₋₄ alkyl, C₁₋₄ alkoxy or C₁₋₄ haloalkyl;
and n is 1 to 6, preferably n is 1, 2, 3 or 4; and
R² is optionally substituted C₁₋₁₂ alkyl, C₂₋₁₂ alkenyl, C₂₋₁₂ alkynyl, C₃₋₁₂ carbocyclyl or C₃₋₁₂ heterocyclyl, wherein the optionally substituted carbocyclyl or heterocyclyl group is optionally fused to one to three unsaturated, partially unsaturated or fully saturated five to seven membered rings containing zero to three heteroatoms;
each substitutable carbon atom in R², including the optional fused ring, is optionally and independently substituted by one or more of C₁₋₁₂ alkyl, C₃₋₁₂ cycloalkyl, C₃₋₁₂ heterocycloalkyl, C₃₋₁₂ aryl, C₃₋₁₂ heteroaryl halogen, C₁₋₁₂ haloalkyl, OR⁹, SR⁹, NO₂, CN, NR⁹R⁹, NR⁹COR⁹, NR⁹CONR⁹R⁹, NR⁹COR⁹, NR⁹CO₂R⁹, CO₂R⁹, COR⁹, CONR⁹R⁹, S(O)₂R⁹, SONH₂, S(O)R⁹, SO₂NR⁹R⁹, NR⁹S(O)₂R⁹, wherein each R⁹ may be the same or different and is as defined below and wherein:
the C₁₋₁₂ alkyl group optionally incorporates one or two insertions selected from the group consisting of —O—, —C(O)—, —N(R⁹)—, —S(O)— and —S(O₂)—, wherein each R⁹ may be the same or different and is as defined above;
the C₁₋₁₂ alkyl, C₃₋₁₂ cycloalkyl, C₃₋₁₂ heterocycloalkyl, C₃₋₁₂ aryl, or C₃₋₁₂ heteroaryl groups are optionally substituted by one or more of halogen, C₁₋₁₂ haloalkyl, OR⁹, SR⁹, NO₂, CN, NR⁹R⁹, NR⁹COR⁹, NR⁹CONR⁹R⁹, NR⁹COR⁹, NR⁹CO₂R⁹, CO₂R⁹, COR⁹, CONR⁹R⁹, S(O)₂R⁹, SONH₂, S(O)R⁹, SO₂NR⁹R⁹, NR⁹S(O)₂R⁹, wherein each R⁹ may be the same or different and is as defined below; and
the C₃₋₁₂ cycloalkyl, C₃₋₁₂ heterocycloalkyl, C₃₋₁₂ aryl, or C₃₋₁₂ heteroaryl groups are optionally substituted by one or more C₁₋₁₂ alkyl groups;
each saturated carbon in R², including the optional fused ring, is further optionally and independently substituted by ═O, ═S, NNR⁹R⁹, —N—OR⁹, ═NNHCOR⁹, ═NNHCO₂R⁹, ═NNSO₂R⁹, or ═NR⁹, wherein each R⁹ may be the same or different and is as defined below; and
each substitutable nitrogen atom in R² is optionally substituted by R¹⁰, COR⁹, SO₂R⁹ or CO₂R⁹ wherein each R⁹ and R¹⁰ may be the same or different and is as defined below;
wherein two R⁹ in NR⁹₂ may optionally form a partially saturated, unsaturated or fully saturated three to seven membered ring containing one to three heteroatoms, optionally and independently substituted by one or more of C₁₋₆ alkyl, halogen, C₁₋₆ haloalkyl, OR¹¹, SR¹¹, NO₂, CN, NR¹¹R¹¹, NR¹¹COR¹¹, NR¹¹CONR¹¹R¹¹, NR¹¹COR¹¹, NR¹¹CO₂R¹¹, CO₂R¹¹, COR¹¹, CONR¹¹₂, S(O)₂R¹¹, SONR¹¹₂, S(O)R¹¹, SO₂NR¹¹R¹¹, NR¹¹S(O)₂R¹¹,
wherein the C₁₋₆ alkyl group optionally incorporates one or two insertions selected from the group consisting of —O—, —N(R¹¹)—, —S(O)— and —S(O₂)—, wherein each R¹¹ may be the same or different and is as defined below;
wherein R¹¹ is hydrogen, C₁₋₆ alkyl, or C₁₋₆ haloalkyl;
wherein R⁹ is hydrogen, C₁₋₁₂ alkyl or C₃₋₁₂ aryl, optionally substituted by one or more of C₁₋₄ alkyl, halogen, C₁₋₄ haloalkyl, OR¹², SR¹², NO₂, CN, NR¹²R¹², NR¹²COR¹², NR¹²CONR¹²R¹², NR¹²COR¹², NR¹²CO₂R¹², CO₂R¹², COR¹², CONR¹²₂, S(O)₂R¹², SONH₂, S(O)R , SO¹²NR¹²R¹², NR¹²S(O)₂R¹², wherein the C₁₋₁₂ alkyl group optionally incorporates one or two insertions selected from the group consisting of —O—, —N(R¹²)—, —S(O)— and —S(O₂)—, wherein each R¹² may be the same or different and is as defined below;
wherein R¹⁰ is C₁₋₁₂ alkyl or C₃₋₁₂ aryl, optionally substituted by one or more of C₁₋₄ alkyl, halogen, C₁₋₄ haloalkyl, OR¹², SR¹², NO₂, CN, NR¹²R¹², NR¹²COR¹², NR¹²CONR¹²R¹², NR¹²COR¹², NR¹²CO₂R¹², CO₂R¹², COR¹², CONR¹²₂, S(O)₂R¹², SONH₂, S(O)R¹², SO₂NR¹²R¹², NR¹²S(O)₂R¹², wherein the C₁₋₁₂ alkyl group optionally incorporates one or two insertions selected from the group consisting of —O—, —N(R¹²)—, —S(O)— and —S(O₂)—, wherein each R¹² may be the same or different and is as defined below;
wherein R¹² is hydrogen, C₁₋₄ alkyl, or C₁₋₄ haloalkyl.

4. A compound as claimed in claim 1 wherein X is NR^{3;} O, S or (CR²²-R²²) wherein R²² is independently one or more of halogen, C₁₋₁₂ alkyl, C₂₋₁₂ alkenyl, C₂₋₁₂ alkynyl, C₁₋₁₂ haloalkyl, C₆₋₁₂ carbocyclyl, C₅₋₁₂ heterocyclyl, (CH₂)ₙOR⁵, (CH₂)ₙNR⁵₂, OR⁵, SR⁵, NO₂, CN, NR⁵₂, NR⁵COR⁵, NR⁵COR⁵, NR⁵CONR⁵ₛ, NR⁵COR⁵, NR⁵CO₂R⁵, CO₂R⁵, COR⁵, CONR⁵₂, S(O)₂R⁵, SONR⁵₂, S(O)R⁵, SO₂NR⁵₂, or NR⁵S(O)₂R⁵ wherein each R⁵ may be the same or different and is as defined above; and
wherein n is 1 to 6, preferably n is 1, 2, 3 or 4;
and wherein R³ is a lone electron pair, hydrogen, C₁₋₁₂ alkyl, C₂₋₁₂ alkenyl, C₂₋₁₂ alkynyl, C₃₋₁₂ carbocyclyl or C₃₋₁₂ heterocyclyl, each of which is optionally substituted, wherein:
the optionally substituted carbocyclyl or heterocyclyl group is optionally fused to one to three unsaturated, partially unsaturated or fully saturated five to seven membered rings containing zero to three heteroatoms,
each substitutable carbon atom in R³, including the optional fused ring, is optionally and independently substituted by one or more of C₁₋₁₂ alkyl, C₃₋₁₂ cycloalkyl, C₃₋₁₂ heterocycloalkyl, C₃₋₁₂ aryl, C₃₋₁₂ heteroaryl halogen, C₁₋₁₂ haloalkyl, OR¹³, SR¹³, NO₂, CN, NR¹²R¹³, NR¹³COR¹³, NR¹³CONR¹³R¹³, NR¹³COR¹³, NR¹³CO₂R¹³, CO₂R¹³, COR¹³, CONR¹³R¹³, S(O)₂R¹³, SONH₂, S(O)R¹³, SO₂NR¹³R¹³, NR¹³S(O)₂R¹³, wherein each R may be the same or different and is as defined above and wherein:
the C₁₋₁₂ alkyl group optionally incorporates one or two insertions selected from the group consisting of —O—, —C(O)—, —N(R¹³)—, —S(O)— and —S(O₂)—, wherein each R¹³ may be the same or different and is as defined above;
the C₁₋₁₂ alkyl, C₃₋₁₂ cycloalkyl, C₃₋₁₂ heterocycloalkyl, C₃₋₁₂ aryl, or C₃₋₁₂ heteroaryl groups are optionally substituted by one or more of halogen, C₁₋₁₂ haloalkyl, OR¹³, SR¹³, NO₂, CN, NR¹³R¹³, NR¹³COR¹³, NR¹³CONR¹³R¹³, NR¹³COR¹³, NR¹³CO₂R¹³, CO₂R¹³, COR¹³, CONR¹³R¹³, S(O)₂R¹³, SONH₂, S(O)R¹³, SO₂NR¹³R¹³, NR¹³S(O)₂R¹³, wherein each R¹³ may be the same or different and is as defined below; and
the C₃₋₁₂ cycloalkyl, C₃₋₁₂ heterocycloalkyl, C₃₋₁₂ aryl, or C₃₋₁₂ heteroaryl groups are optionally substituted by one or more C₁₋₁₂ alkyl groups;
each saturated carbon in R², including the optional fused ring, is further optionally and independently substituted by ═O, ═S, NNR¹³R¹³, ═N—OR¹³, ═NNHCOR¹³, ═NNHCO₂R¹³, ═NNSO₂R¹³, or ═NR¹³, wherein each R¹³ may be the same or different and is as defined below; and
each substitutable nitrogen atom in R¹³ is optionally substituted by R¹⁴, COR¹³, SO₂R¹³or CO₂R¹³ wherein each R¹³ and R¹⁴ may be the same or different and is as defined below;
wherein two R¹³ in NR¹³₂ may optionally form a partially saturated, unsaturated or fully saturated three to seven membered ring containing one to three heteroatoms, optionally and independently substituted by one or more of C₁₋₆ alkyl, halogen, C₁₋₆ haloalkyl, OR¹⁵, SR¹⁵, NO₂, CN, NR¹⁵R¹⁵, NR¹⁵COR¹⁵, NR¹⁵CONR¹⁵R¹⁵, NR¹⁵COR¹⁵, NR¹⁵CO₂R¹⁵, CO₂R¹⁵, COR¹⁵, CONR¹⁵₂, S(O)₂R¹⁵, SONR¹⁵₂, S(O)R¹⁵, SO₂NR¹⁵R⁵, NR¹⁵S(O)₂R¹⁵,
wherein the C₁₋₆ alkyl group optionally incorporates one or two insertions selected from the group consisting of —O—, —N(R¹⁵)—, —S(O)— and —S(O₂)—, wherein each R¹⁵ may be the same or different and is as defined below;
wherein R¹⁵ is hydrogen, C₁₋₆ alkyl, or C₁₋₆haloalkyl;
wherein R¹³ is hydrogen, C₁₋₁₂ alkyl or C₃₋₁₂ aryl, optionally substituted by one or more of C₁₋₄ alkyl, halogen, C₁₋₄ haloalkyl, OR¹⁶, SR¹⁶, NO₂, CN, NR¹⁶R¹⁶, NR¹⁶COR¹⁶, NR¹⁶CONR¹⁶R¹⁶, NR¹⁶COR¹⁶, NR¹⁶CO₂R¹⁶, CO₂R¹⁶, COR¹⁶, CONR¹⁶₂, S(O)₂R¹⁶, SONH₂, S(O)R¹⁶, SO₂NR¹⁶R¹⁶, NR¹⁶S(O)₂R¹⁶, wherein the C₁₋₁₂ alkyl group optionally incorporates one or two insertions selected from the group consisting of —O—, —N(R¹⁶)—, —S(O)— and —S(O₂)—, wherein each R¹⁶ may be the same or different and is as defined below;
wherein R¹⁴ is C₁₋₁₂ alkyl or C₃₋₁₂ aryl, optionally substituted by one or more of C₁₋₄ alkyl, halogen, C₁₋₄ haloalkyl, OR¹⁶, SR¹⁶, NO₂, CN, NR¹⁶R¹⁶, NR¹⁶COR¹⁶, NR¹⁶CONR¹⁶R¹⁶, NR¹⁶COR¹⁶, NR¹⁶CO₂R¹⁶, CO₂R¹⁶, COR¹⁶, CONR¹⁶₂, S(O)₂R¹⁶, SONH₂, S(O)R¹⁶, SO₂NR¹⁶R¹⁶, NR¹⁶S(O)₂R¹⁶, wherein the C₁₋₁₂ alkyl group optionally incorporates one or two insertions selected from the group consisting of —O—, —N(R¹⁶)—, —S(O)— and —S(O₂)—, wherein each R¹⁶ may be the same or different and is as defined below;
wherein R¹⁶ is hydrogen, C₁₋₄ alkyl, or C₁₋₄ haloalkyl;
wherein when X is NR², R² and R³ can form a 3 to 12 membered heterocyclyl ring, more preferably a 5, 6, 7, 8, 9, 10, 11 or 12 membered ring, wherein said ring can be partially saturated, unsaturated or fully saturated containing one to three heteroatoms; wherein the heterocyclylic group formed by R² and R³ can be optionally fused to one to three unsaturated, partially saturated or fully saturated 5 to 7 membered rings containing zero to three heteroatoms, any of said rings being optionally and independently substituted with one or more of C₁₋₆ alkyl, halogen, C₁₋₆ haloalkyl, OR²², SR²², NO₂, CN, NR²²R²², NR²²COR²², NR²²CONR²²R²², NR²²COR²², NR²²CO₂R²², CO₂R²², COR²², CONR²², S(O)₂R²², SONR₂₂, S(O)R²², SO₂₂NR²²R²², NR²²S(O)₂R²², wherein the C₁₋₆ alkyl group optionally incorporates one or two insertions from —O—, —N(R²²)—, —S(O)— and —S(O₂)— and wherein each R²² may be the same or different.

5. A compound as claimed in claim 1 wherein R⁴ is a six-membered carbocyclyl group or a five or six-membered heterocyclyl group containing from 1 to 4 heteroatoms independently selected from N, S or O, wherein the optionally substituted six-membered carbocyclyl or five or six-membered heterocyclyl group is optionally fused to a partially saturated, unsaturated or fully saturated five to seven membered ring containing zero to three heteroatoms, and each substitutable carbon or hetero-atom in R⁴ including the optional fused ring, is optionally and independently substituted by one or more of halogen, C₁₋₁₂ alkyl, C₂₋₁₂ alkenyl, C₂₋₁₂ alkynyl, C₁₋₁₂ haloalkyl, C₃₋₁₂ carbocyclyl, C₃₋₁₂ heterocyclyl, (CH₂)ₙOR¹⁷, (CH₂)ₙNR¹⁷₂, OR¹⁷, SR¹⁷, NO₂, CN, NR¹⁷₂, NR¹⁷COR¹⁷, NR¹⁷CONR¹⁷₂, NR¹⁷COR¹⁷, NR¹⁷CO₂R¹⁷, CO₂R¹⁷, COR¹⁷, CONR¹⁷₂, S(O)₂R¹⁷, SONR¹⁷₂, S(O)R¹⁷, SO₂NR¹⁷₂, or NR¹⁷S(O)₂R¹⁷, wherein the C₁₋₁₂ alkyl group optionally contains one or more insertions selected from —O—, —N(R¹²)— —S—, —S(O)— and —S(O₂)—; and each saturated carbon in the optional fused ring is further optionally and independently substituted by ═O, ═S, NNR¹⁸₂, ═N—OR¹⁸, ═NNR¹⁸COR¹⁸, ═NNR¹⁸CO₂R¹⁸, ═NNSO₂R¹⁸, or ═NR¹⁸; and each substitutable nitrogen atom in R⁴ is optionally substituted by R¹⁹, COR¹⁹; SO₂R¹⁹ or CO₁R¹⁹ wherein n is 1 to 6, preferably n is 1, 2 or 3; preferably, wherein each substitutable carbon or hetero-atom in R⁴ is optionally and independently substituted by one or more of C₁₋₆ alkyl, OR²⁰, SR²⁰, NO₂, CN, NR²⁰₂, NR²⁰COR²⁰, NR²⁰CONR²⁰₂, NR²⁰COR²⁰, NHCO₂R²⁰, CO₂R²⁰, COR²⁰, CONR²⁰₂, S(O)₂R²⁰, SONR²⁰₂, S(O)R²⁰, SO₂NR²⁰, or NR²⁰S(O)₂R²⁰;
wherein R²⁰ is hydrogen, C₁₋₆ alkyl, or C₁₋₆ haloalkyl;
wherein R¹⁷ is hydrogen, C₁₋₁₂ alkyl, C₃₋₁₂ carbocyclyl or C₃₋₁₂ heterocyclyl, optionally substituted by one or more of C₁₋₆ alkyl, C₃₋₁₂ carbocyclyl, C₃₋₁₂ heterocyclyl, halogen, C₁₋₆ haloalkyl, OR²¹, SR²¹, NO₂, CN, NR²¹R²¹, NR²¹COR²¹, NR²¹CONR²¹R²¹, NR²¹COR²¹, NR²¹CO₂R²¹, CO₂R²¹, COR²¹, CONR²¹₂, S(O)₂R²¹, SONR²¹₂, S(O)R²¹, SO₂NR²¹R²¹, NR²¹S(O)₂R²¹, wherein the C₁₋₁₂ alkyl group optionally incorporates one or two insertions selected from the group consisting of —O—, —N(R²¹)—, —S(O)— and —S(O₂)—, wherein each R²¹ may be the same or different and is as defined below;
wherein two R¹⁷ in NR¹⁷₂ may optionally form a partially saturated, unsaturated or fully saturated three to seven membered ring containing one to three heteroatoms, optionally and independently substituted by one or more of C₁₋₆ alkyl, halogen, C₁₋₆ haloalkyl, OR²¹, SR²¹, NO₂, CN, NR²¹R²¹, NR²¹COR²¹, NR²¹CONR²¹R²¹, NR²¹COR²¹, NR²¹CO₂R²¹, CO₂R²¹, COR²¹, CONR²¹₂, S(O)₂R²¹, SONR²¹₂, S(O)R²¹, SO₂NR²¹R²¹, NR²¹S(O)₂R²¹, wherein the C₁₋₆ alkyl group optionally incorporates one or two insertions selected from the group consisting of —O—, —N(R²¹)—, —S(O)— and —S(O₂)—, wherein each R may be the same or different and is as defined below;
wherein R¹⁸ is hydrogen, C₁₋₁₂ alkyl, C₃₋₁₂ carbocyclyl or C₃-1₂ heterocyclyl, optionally substituted by one or more of C₁₋₆ alkyl, halogen, C₁₋₆ haloalkyl, OR²¹, SR²¹, NO₂, CN, NR²¹R²¹, NR²¹COR²¹, NR²¹CONR²¹R²¹, NR^{21 COR}²¹, NR²¹CO₂R²¹, CO₂R²¹, COR²¹, CONR²¹₂, S(O)₂R²¹, S(O)R²¹, SO₂NR²¹R²¹, NR S(O)₂R²¹, wherein the C₁₋₁₂ alkyl group optionally incorporates one or two insertions selected from the group consisting of —O—, —N(R²¹)—, —S(O)— and —S(O₂)—, wherein each R²¹ may be the same or different and is as defined below;
wherein R¹⁹ is hydrogen, C₆₋₁₂ aryl, C₁₋₆ alkyl or C₁₋₆ haloalkyl;
wherein R²¹ is hydrogen, C₁₋₆ alkyl, or C₁₋₆ haloalkyl.

6. A compound as claimed in claim 1 wherein R¹ is an optionally substituted five or six membered carbocyclyl or heterocyclyl group selected from optionally substituted phenyl, acridine, benzimidazole, benzofuran, benzothiophene, benzoxazole, benzothiazole, cyclohexyl furan, imidazole, indole, isoindole, isoquinoline, isoxazole, isothiazole, morpholine, napthaline, oxazole, phenazine, phenothiazine, phenoxazine, piperazine, piperidine, pyrazole, pyridazine, pyridine, pyrrole, quinoline, quinolizine, tetrahydrofuran, tetrazine, tetrazole, thiophene, thiazole, thiomorpholine, thianaphthalene, thiopyran, triazine, triazole or trithiane.

7. A compound as claimed in claim 1 wherein R¹ is a group of formula (II), wherein X is a group NR³, Y is absent and one or more of R² and R³ is hydrogen, alkyl or cycloalkyl.

8. A compound as claimed in claim 7 wherein the group of formula (II) is an alkylamino or cycloalkylamino group preferably selected from optionally substituted methylamino, ethylamino, propylamino, isopropylamino, butylamino, cyclobutylamino, pentylamino, cyclopentylamino, hexylamino, cyclohexylamino, heptylamino, cycloheptylamino, octylamino and cyclooctylamino.

9. A compound as claimed in claim 1 wherein R¹ is substituted with one or more of OR²⁴, halogen, C₁₋₆ alkyl, C₂₋₆ alkenyl, C₂₋₆ alkynyl, C₁₋₆haloalkyl, C₁₋₆alkylaryl, C₁₋₆alkylheterocyclyl, (CH₂)ₙOR²⁴, (CH₂)ₙNR²⁴₂, SR²⁴, NO₂, CN, NR₂₄₂, CO₂R²⁴, NR²⁴C(O)R²⁴, NR²⁴S(O)₂R²⁴, COR²⁴, CONR²⁴₂, S(O)₂R²⁴, S(O)R²⁴ or SO₂NR²⁴₂;
wherein R²⁴ is hydrogen, C₁₋₄ alkyl or C₆₋₁₂ aryl preferably phenyl, or C₅₋₁₂ heterocyclyl preferably pyridine, and n is 1, 2, 3, 4, 5 or 6;
wherein two R²⁴ in NR²⁴₂ may optionally form a partially saturated, unsaturated or fully saturated three to seven membered ring containing one to three heteroatoms, said ring is preferably independently substituted with one or more of halogen, C₁₋₁₂ alkyl, C₂₋₁₂ alkenyl, C₂₋₁₂ alkynyl, C₁₋₁₂ haloalkyl, C₃₋₁₂ carbocyclyl, C₃₋₁₂ heterocyclyl, OR²⁵, SR²⁵, NO₂, CN, NR²⁵₂, NR²⁵COR²⁵, NR²⁵CONR²⁵₂, NR²⁵COR²⁵, NR²⁵CO₂R²⁵, CO₂R²⁵, COR²⁵, CONR²⁵₂, S(O)₂R²⁵, SONR²⁵₂, S(O)R²⁵, SO₂NR²⁵, or NR²⁵S(O)₂R²⁵; and each saturated carbon in the optional ring is further optionally and independently substituted by ═O, ═S, NNR²⁶₂, ═N—OR²⁶, ═NNR²⁶COR²⁶, ═NNR²⁶CO₂R²⁶, ═NNSO₂R²⁶, or ═NR²⁶; and each substitutable nitrogen atom is optionally substituted by R²⁷, COR²⁷, SO₂R²⁷ or CO₂R²⁷;
wherein R²⁵ is hydrogen, C₁₋₁₂ alkyl, C₆₋₁₂ carbocyclyl or C₅₋₁₂ heterocyclyl, optionally substituted by one or more of C₁₋₆ alkyl, halogen, C₁₋₆ haloalkyl, OR²⁸, SR²⁸, NO₂, CN, NR²⁸R²⁸, NR²⁸COR²⁸, NR²⁸CONR²⁸R²⁸, NR²⁸COR²⁸, NR²⁸CO₂R²⁸, CO₂R²⁸, COR²⁸, CONR²⁸₂, S(O)₂R²⁸, SONR²⁸₂, S(O)R²⁸, SO₂NR²⁸R²⁸, NR²⁸S(O)₂R²⁸, wherein the C₁₋₁₂ alkyl group optionally incorporates one or two insertions selected from the group consisting of —O—, —N(R²⁸)—, —S(O)— and —S(O₂)—, wherein each R²⁸ may be the same or different and is as defined below;
wherein R²⁶ is hydrogen, C₁₋₁₂ alkyl, C₆₋₁₂ carbocyclyl or C₅₋₁₂ heterocyclyl, optionally substituted by one or more of C₁₋₆ alkyl, halogen, C₁₋₆ haloalkyl, OR²⁸, SR²⁸, NO₂, CN, NR²⁸R²⁸, NR²⁸COR²⁸, NR²⁸CONR²⁸R²⁸, NR²⁸COR²⁸, NR²⁸CO₂R²⁸, CO₂R²⁸, COR²⁸, CONR²⁸₂, S(O)₂R²⁸, S(O)R²⁸, SO₂NR²⁸R²⁸, NR²⁸S(O)₂R²⁸, wherein the C₁₋₁₂ alkyl group optionally incorporates one or two insertions selected from the group consisting of —O—, —N(R²⁸)—, —S(O)— and —S(O₂)—, wherein each R²⁸ may be the same or different and is as defined below;
wherein R²⁷ is hydrogen, C₁₋₆ alkyl, C₁₋₆ haloalkyl or C₆₋₁₂ aryl;
wherein R²⁸ is hydrogen, C₁₋₆ alkyl, or C₁₋₆ haloalkyl.

10. A compound as claimed in claim 1 wherein R⁴ is selected from phenyl, cyclohexyl, acridine, benzimidazole, benzofuran, benzothiophene, benzoxazole, benzothiazole, indole, isoindole, indolizine, indazole, isoindole, isoquinoline, morpholine, napthalene, phenazine, phenothiazine, phenoxazine, piperazine, piperidine, pyridazine, pyridine, pyridinyl, pyrimidinyl, pyrazinyl, pyridazinyl, quinoline, quinolizine, tetrazine, thiomorpholine, thianaphthalene, thiopyran, triazine, trithiane, furan, imidazole, isoxazole, isothiazole, oxazole, oxadiazole, oxathiazole, pyrazole, pyrrole, tetrazole, thiophene, thiadiazole, thiatriazole, thiazole or triazole, wherein each substitutable carbon or hetero-atom in R⁴ is optionally and independently substituted by one or more of C₁₋₆ alkyl, OR²⁰, SR²⁰, NO₂, CN, NR²⁰₂, NR²⁰COR²⁰, NR²⁰CONR²⁰₂, NR²⁰COR²⁰, NHCO₂R²⁰, CO₂R²⁰, COR²⁰, CONR²⁰₂, S(O)₂R²⁰, SONR²⁰₂, S(O)R²⁰, SO₂NR²⁰₂, or NR²⁰S(O)₂R²⁰;
wherein R²⁰ is hydrogen, C₁₋₆ alkyl, or C₁₋₆ haloalkyl.

11. A compound as claimed in claim 1 wherein R⁴ is a six-membered carbocyclyl or heterocyclyl group optionally substituted with one or more of OR²⁹, NR²⁹₂, SR²⁹, (CH₂)ₙOR²⁹, (CH₂)ₙNR²⁹₂, halogen, C₁₋₆ alkyl, C₂₋₆ alkenyl, C₂₋₆ alkynyl, haloalkyl, NO₂, CN, NR²⁹C(O)R²⁹, NR²⁹S(O)₂R²⁹, CO₂R²⁹, COR²⁹, CONR²⁹₂, S(O)₂R²⁹, S(O)R²⁹ or SO₂NR²⁹₂;
wherein R²⁹ is hydrogen, C₁₋₄ alkyl, C₅₋₁₂ heterocyclyl or C₆₋₁₂ aryl preferably phenyl, and n is 1, 2, 3, 4, 5 or 6;
wherein two R²⁹ in NR²⁹₂ may optionally form a partially saturated, unsaturated or fully saturated five to seven membered ring containing one to three heteroatoms, optionally and independently substituted with one or more of halogen, C₁₋₁₂ alkyl, C₂₋₁₂ alkenyl, C₂₋₁₂ alkynyl, C₁₋₁₂ haloalkyl, C₆₋₁₂ carbocyclyl, C₅₋₁₂ heterocyclyl, OR³⁰, SR³⁰, NO₂, CN, NR³⁰₂, NR³⁰COR³⁰, NR³⁰CONR³⁰₂, NR³⁰COR³⁰, NR³⁰CO₂R³⁰, CO₂R³⁰, COR³⁰, CONR³⁰₂, S(O)₂R³⁰, SONR³⁰₂, S(O)R³⁰, SO₂NR³⁰₂, or NR³⁰S(O)₂R³⁰; and each saturated carbon in the optional ring is further optionally and independently substituted by ═O, ═S, NNR³¹₂, ═N—OR³¹, ═NNR³¹COR³¹, ═NNR³¹CO₂R³¹, ═NNSO₂R³¹, or ═NR³¹; and each substitutable nitrogen atom is optionally substituted by R³², COR³², SO₂R³² or CO₂R³²;
wherein R³⁰ is hydrogen, C₁₋₁₂ alkyl, C₆₋₁₂ carbocyclyl or C₅₋₁₂ heterocyclyl, optionally substituted by one or more of C₁₋₆ alkyl, halogen, C₁₋₆ haloalkyl, OR³³, SR³³, NO₂, CN, NR³³R³³, NR³³ CR³³, NR³³CONR³³R³³, NR³³COR³³, NR³³CO₂R³³, CO₂R³³, COR³³, CONR³³₂, S(O)₂R³³, SNR³³₂, S(O)R³³, SO₂NR³³R³³, NR³³S(O)₂R³³, wherein the C₁₋₁₂ alkyl group optionally incorporates one or two insertions selected from the group consisting of —O—, —N(R³³)—, —S(O)— and —S(O₂)—. wherein each R³³ may be the same or different and is as defined below;
wherein R³¹ is hydrogen, C₁₋₁₂ alkyl, C₆₋₁₂ carbocyclyl or C₅₋₁₂ heterocyclyl, optionally substituted by one or more of C₁₋₆ alkyl, halogen, C₁₋₆ haloalkyl, OR³³, SR³³, NO₂, CN, NR³³R³³, NR³³COR³³, NR³³CONR³³R³³, NR³³COR³³, NR³³CO₂R³³, CO₂R³³, COR³³, CONR³³₂, S(O)₂R³³, S(O)R³³, SO₂NR³³R³³, NR³³S(O)₂R³³, wherein the C₁₋₁₂ alkyl group optionally incorporates one or two insertions selected from the group consisting of —O—, —N(R³³)—, —S(O)— and —S(O₂)—, wherein each R²¹ may be the same or different and is as defined below;
wherein R³² is hydrogen, C₆₋₁₂ aryl, C₁₋₆ alkyl or C₁₋₆ haloalkyl;
wherein R³³ is hydrogen, C₁₋₆ alkyl, or C₁₋₆ haloalkyl.

12. A compound as claimed in claim 1 wherein R⁴ is a five-membered heterocyclyl, 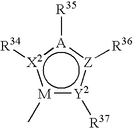
wherein A, X², Y² or Z are independently selected from N, O, C, S and M is C or N, wherein one, two, three or four of A, X², Y², Z and M is other than C;
R³⁴, R³⁵, R³⁶ or R³⁷ are independently selected from a lone electron pair, hydrogen, halogen, C₁₋₁₂ alkyl, C₁₋₁₂ haloalkyl, OR³⁸, SR³⁸, NO₂, CN, NR³⁸₂, NR³⁸COR³⁸, NR³⁸CONR³⁸₂, NR³⁸COR³⁸, NR³⁸CO₂R³⁸, (CH₂)ₙOR³⁸, (CH₂)ₙNR³⁸₂, CO₂R³⁸, COR³⁸, CONR³⁸₂, S(O)₂R³⁸, SONR³⁸₂, S(O)R³⁸, SO₂NR³⁸₂, or NHS(O)₂R³⁸;
wherein n is 1 to 6, preferably n is 1, 2 or 3;
or wherein any two of R³⁴, R³⁵, R³⁶ or R³⁷ may optionally form a partially saturated, unsaturated or fully saturated five to seven membered ring containing zero to three heteroatoms, each saturated carbon in the optional fused ring is further optionally and independently substituted with one or more of halogen, C₁₋₁₂ alkyl, C₂₋₁₂ alkenyl, C₂₋₁₂ alkynyl, C₁₋₁₂ haloalkyl. C₆₋₁₂ carbocyclyl, C₅₋₁₂ heterocyclyl, OR³⁸, SR³⁸, NO₂, CN, NR³⁸ ₂, NR³⁸CONR³⁸₂, NR³⁸COR³⁸, NR³⁸ CO₂R³⁸, (CH₂)ₙOR³⁸, (CH₂)ₙNR³⁸₂, CO₂R³⁸, COR³⁸, CONR³⁸₂, S(O)₂R³⁸ , SONR³⁸₂, S(O)R³⁸, SO₂NR³⁸₂, or NR³⁸S(O)₂R³⁸; and each saturated carbon in the optional fused ring further optionally and independently substituted by ═O, ═S, NNR³⁹₂, ═N—OR³⁹, ═NNR³⁹COR³⁹, ═NNR³⁹CO₂R³⁹, ═NNSO₂R³⁹, or ═NR³⁹; and each substitutable nitrogen atom in R⁴ is optionally substituted by R⁴⁰, COR⁴⁰, SO₂R⁴⁰ or CO₂R⁴⁰;
wherein n is 1 to 6, preferably n is 1, 2 or 3;
wherein R³⁸ is hydrogen, C₁₋₁₂ alkyl, C₆₋₁₂ carbocyclyl or C₅₋₁₂ heterocyclyl, optionally substituted by one or more of C₁₋₆ alkyl, halogen, C₁₋₆ haloalkyl, OR⁴¹, SR⁴¹, NO₂, CN, NR⁴¹R⁴¹, NR⁴¹CONR⁴¹R⁴¹, NR⁴¹COR⁴¹, NR⁴¹CO₂R⁴¹, CO₂R⁴¹, COR⁴¹, CONR⁴¹₂, S(O)₂R⁴¹, SONR⁴¹₂, S(O)R⁴¹, SO₂NR⁴¹R⁴¹, NR⁴¹S(O)₂R⁴¹, wherein the C₁₋₁₂ alkyl group optionally incorporates one or two insertions selected from the group consisting of —O—, —N(R⁴¹)—, —S(O)— and —S(O₂)—, wherein each R⁴¹ may be the same or different and is as defined below;
wherein R³⁹ is hydrogen, C₁₋₁₂ alkyl, carbocyclyl or heterocyclyl, optionally substituted by one or more of C₁₋₆ alkyl, halogen, C₁₋₆ haloalkyl, OR⁴¹, SR⁴¹, NO₂, CN, NR⁴¹R⁴¹, NR⁴COR⁴¹, NR⁴¹CONR⁴¹R⁴¹, NR⁴¹COR⁴¹, NR⁴¹CO₂R⁴¹, CO₂R , COR⁴¹, CONR⁴¹₂, S(O)₂R⁴¹, S(O)R⁴¹, SO₂NR⁴¹R⁴¹, NR⁴¹S(O)₂R⁴¹, wherein the C₁₋₁₂ alkyl group optionally incorporates one or two insertions selected from the group consisting of —O—, —N(R⁴¹)—, —S(O)— and —S(O₂)—, wherein each R⁴¹ may be the same or different and is as defined below;
wherein R⁴⁰ is hydrogen, C₁₋₆ alkyl, C₁₋₆ haloalkyl or C₆₋₁₂ aryl.
wherein R⁴¹ is hydrogen, C₁₋₆ alkyl, or C₁₋₆ haloalkyl.

13. A compound as claimed in claim 12 wherein R⁴ is furan, imidazole, isoxazole, isothiazole, oxazole, oxadiazole, oxatriazole, pyrazole, pyrrole, tetrazole, thiophene, thiadiazole, thiatriazole, thiazole or triazole; and R³⁴, R³⁵, R³⁶ or R³⁷ are independently selected from a lone electron pair, hydrogen, halogen, C₁₋₆ alkyl, C₁₋₆ haloalkyl, OR⁴², SR⁴², CN, NR⁴²₂, NR⁴²COR⁴², CO₂R⁴², COR⁴², CONR⁴²₂, S(O)₂R⁴², or S(O)R⁴²;
wherein R⁴² is hydrogen, C₁₋₄ alkyl, preferably methyl or ethyl or carbocyclyl, preferably phenyl.

14. A compound as claimed claim 1 selected from the group consisting of:

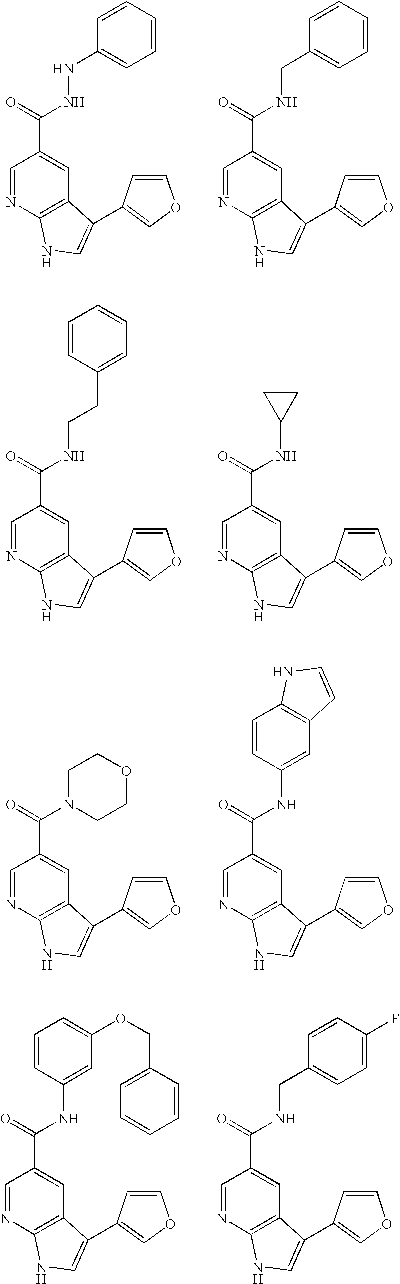

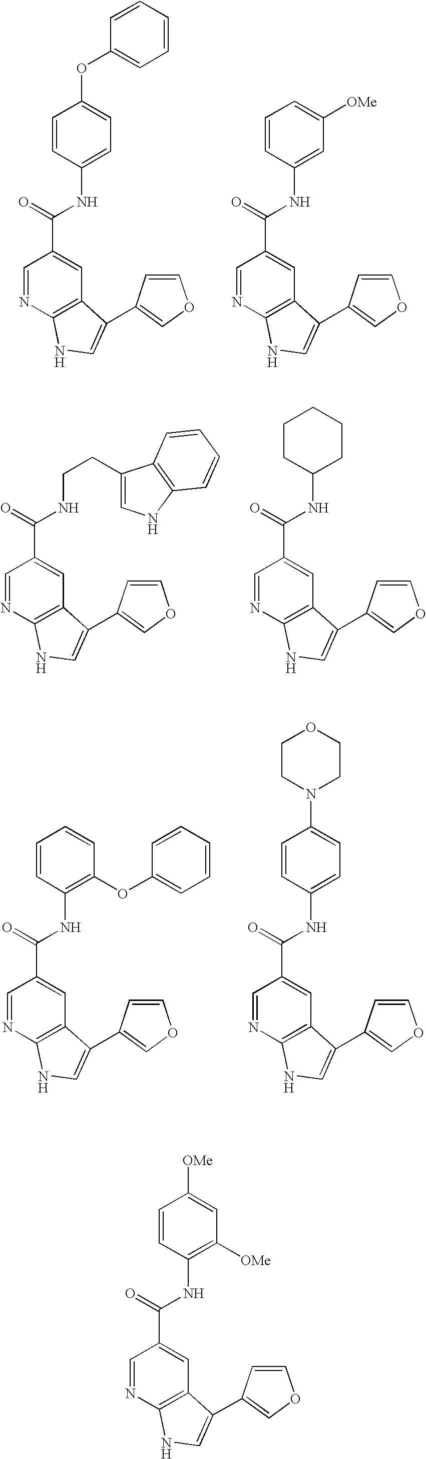

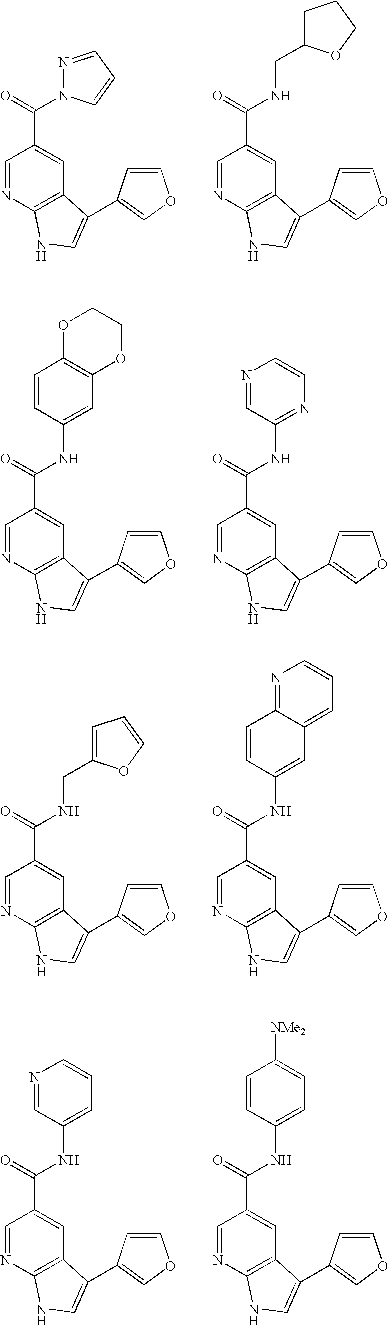

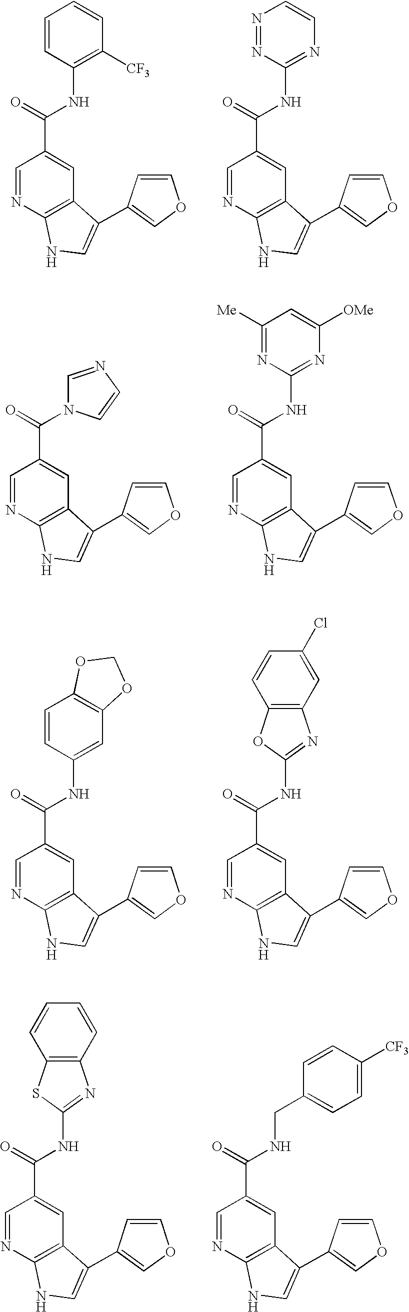

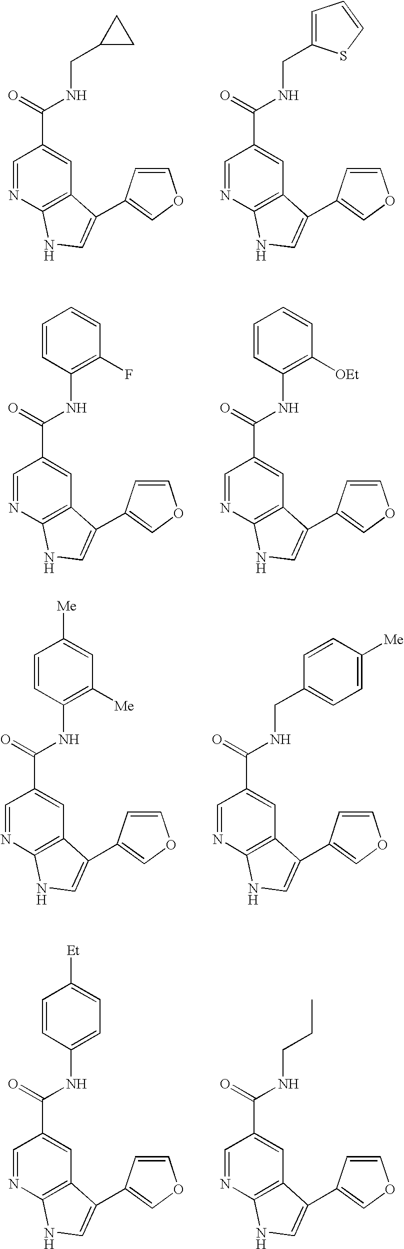

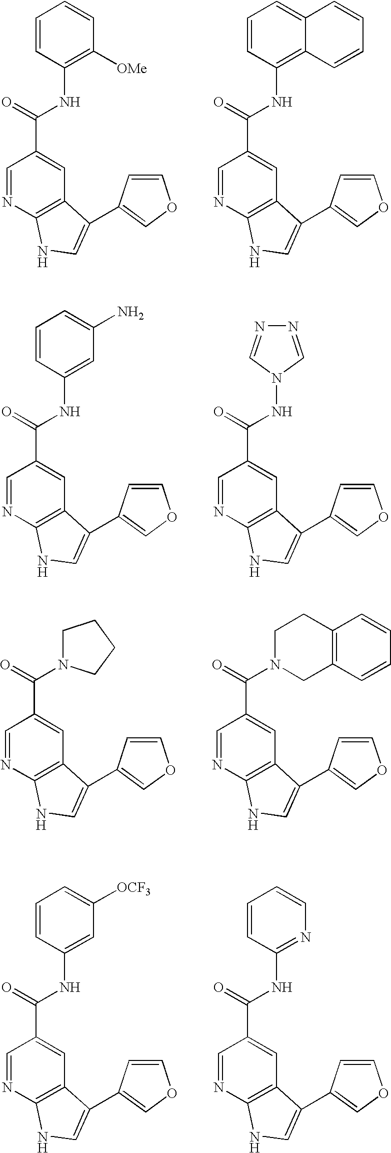

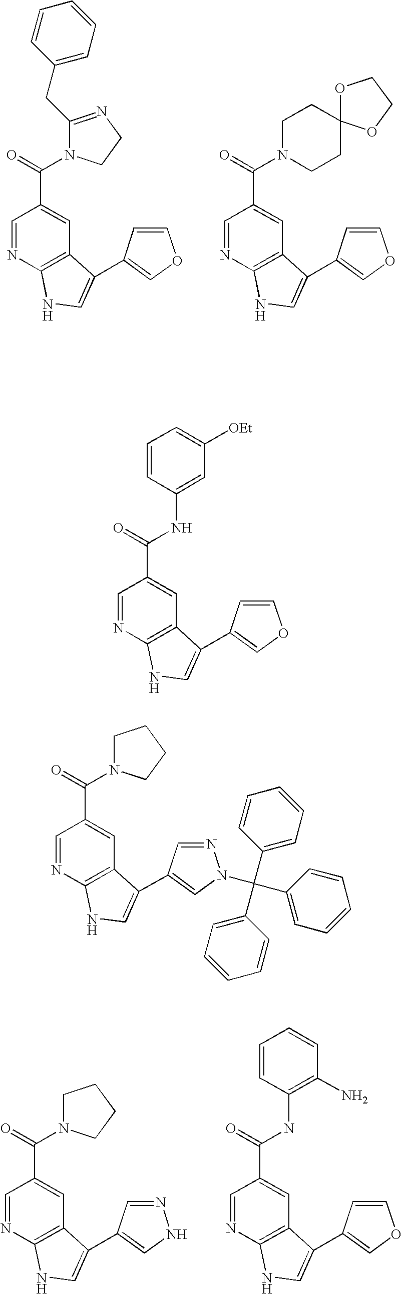

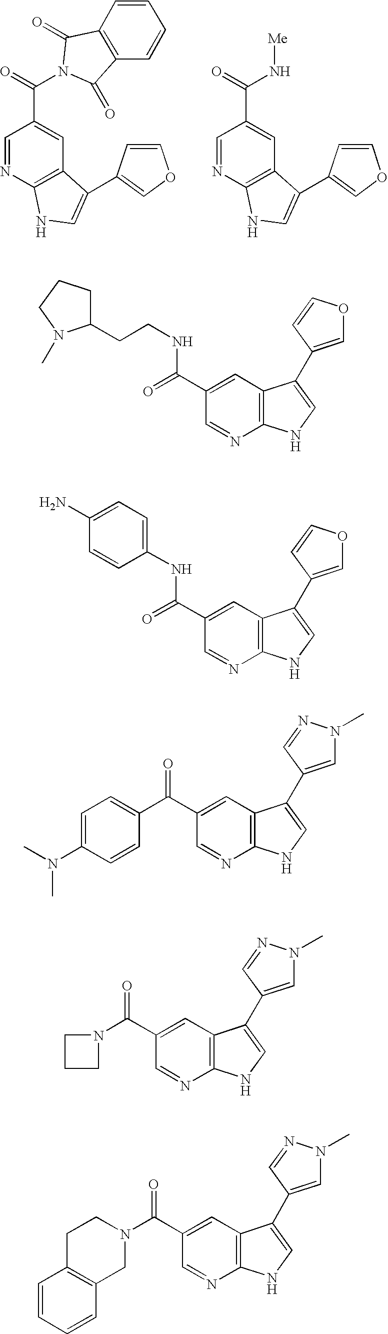

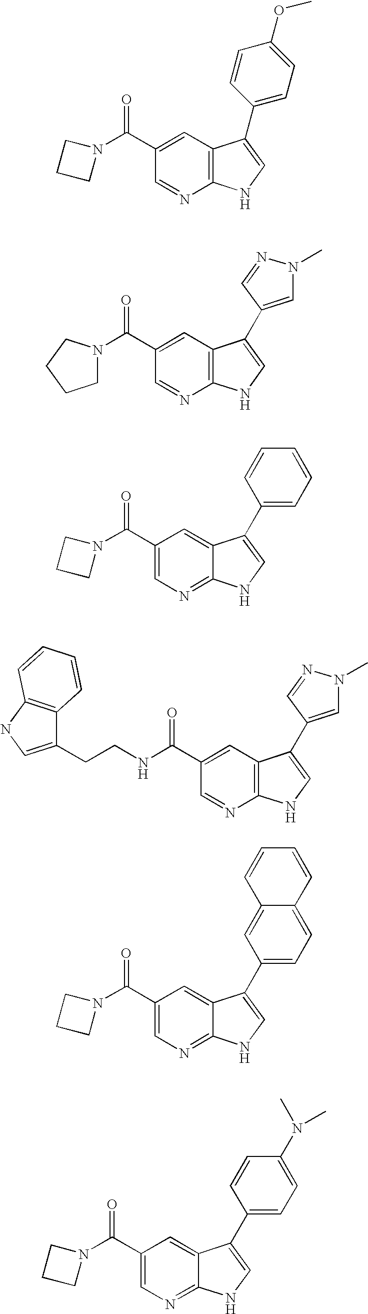

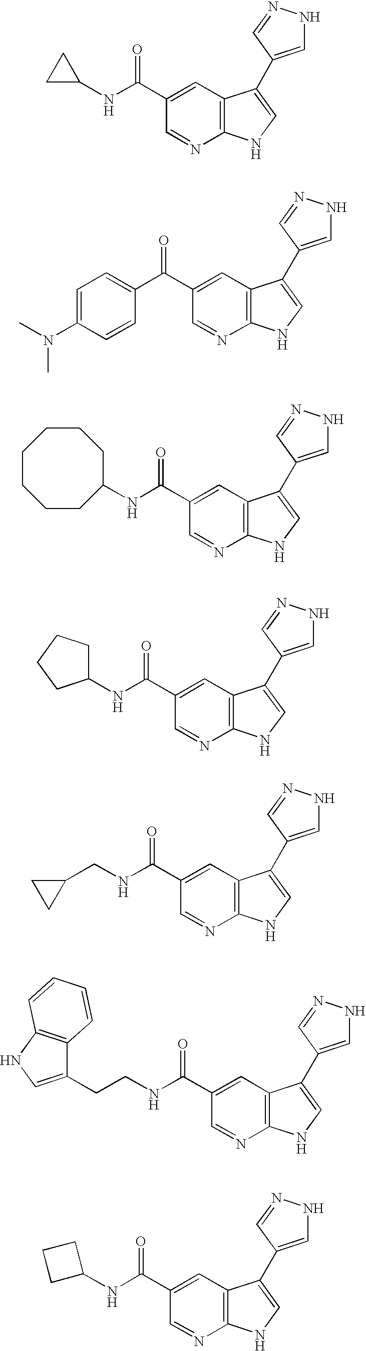

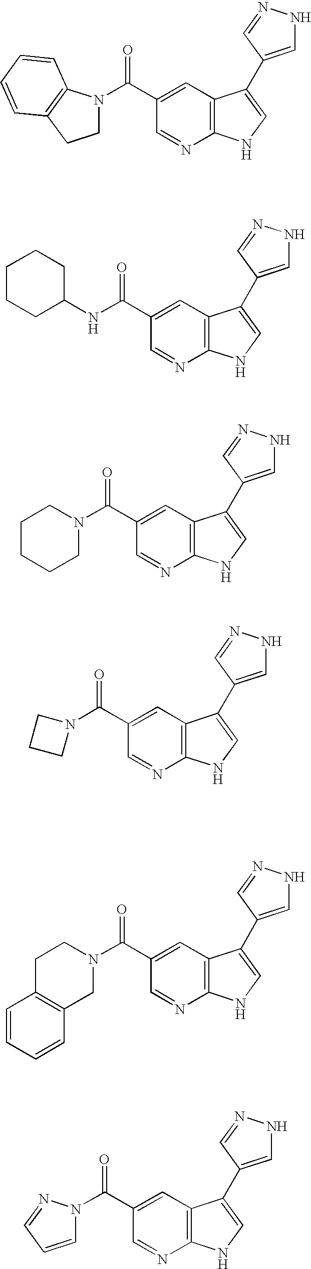

15. A process for the manufacture of a compound of formula (I) wherein R¹ is a group of formula (II) as defined in the claim 1 of the invention comprising the condensation of an intermediate (III) with an intermediate (IV). 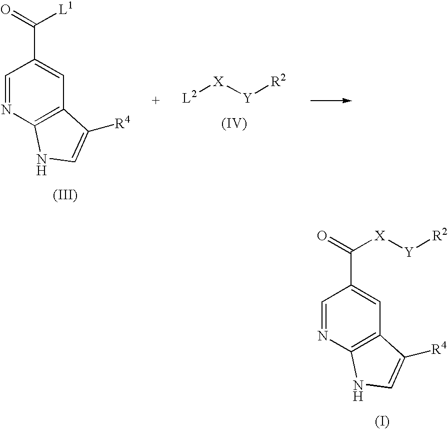
wherein R² and R⁴ are as defined in any one of claims 1 to 14; and wherein each of L¹ and L² is independently a leaving group wherein L¹ and L² together form a condensation product.

16. A process as claimed in claim 15 wherein L¹ is OH, OR⁵⁰, OM, Cl, Br or I wherein R⁵⁰ is C₁₋₆ alkyl, preferably methyl or ethyl and M is Na, Li, K, Ca, Mg or Ba, and L² is hydrogen or M.

17. A compound of formula (III) 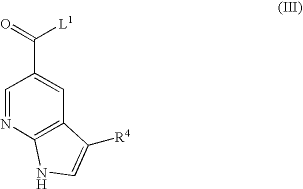
wherein R⁴ is as defined any one of claims 1 to 14
L¹ is OH, OR⁵⁰, OM, Cl, Br, or I
R⁵⁰ is C₁₋₆ alkyl, and
M is Na, Li, K, Ca, Mg, or Ba.

18. A process for the manufacture of a compound of formula (V) comprising removal of group R⁵¹ from an intermediate (VI) 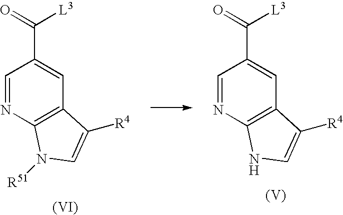
wherein L³ is R¹ or L¹;
R¹ and R⁴ are as defined in any one of claims 1 to 14;
L¹ is as defined in claim 17;
and R⁵¹ is an amino protecting group selected from R⁵²SO₂, R⁵²C(O), R⁵²₃Si, R⁵²OCH₂, (R⁵²)₂NSO₂, (R⁵²)₂NC(O), R⁵²OC(O), R⁵²(R⁵²O)CH, R⁵²CH₂CH₂, R⁵²CH₂, PhC(O)CH₂, CH₂═CH, ClCH₂CH₂, Ph₃C, Ph₂(4-pyridyl)C, Me₂N, HO—CH₂, R⁵²OCH₂, (R⁵²)₃SiOCH₂, (R⁵²O)₂CH, t-BuOC(O)CH₂, Me₂NCH₂, and tetrahydropyranylamine,
wherein R⁵² is C₁₋₆ alkyl or C₆₋₁₂ aryl.

19. A compound of formula (VI) 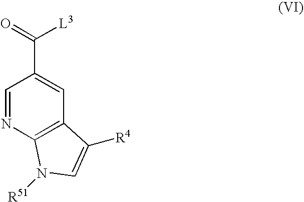
wherein R⁴ is as defined in any one of claims 1 to 14, and
wherein L³ and R⁵¹ are as defined in claim 18.

20. A process for the manufacture of a compound of formula (VI) comprising a) a reaction of a compound of formula (VII) with stannane R⁴—Sn(R⁵³)₃ in the presence of a palladium catalyst or b) reaction of a compound of formula (VII) with boronic acid or ester R⁴—B(OR⁵⁴), in a presence of a suitable palladium catalyst or c) reaction of a comnpound of formula (VII) with silane R⁴—Si(R⁵⁵)₃ in the presence of a palladium catalyst; 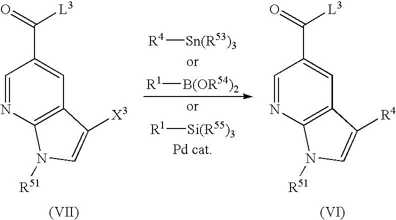
wherein R⁴ is as defined in any one of claims 1 to 14,
L³ is as defined in claim 18;
R⁵¹ is an amino protecting group as defined in claim 18;
X³ is F, Cl, Br I or CF₃SO₃,
and R⁵³ is independently C₁₋₆ alkyl;
R⁵⁴ is independently hydrogen or C₁₋₆ alkyl or wherein two R⁵⁴ groups together optionally form a five, six or seven membered ring with the boron and oxygen atoms, wherein the ring is optionally substituted with one or more C₁₋₆ alkyl group.
and R⁵⁵ is independently C₁₋₆ alkyl, F, or OH.

21. A process as claimed in claim 20 wherein the catalyst is one or more selected from (PPh₃)₂PdCl₂, (PPh₃)₄Pd, Pd(OAc)₂, [PdCl(η³-C₃H₅]₂, Pd₂(dba)₂, Pd(dba)₂(dba=dibenzylidenacetone), and Pd/P(t-Bu)₃.

22. A compound of formula (VII) 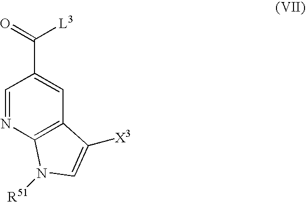
wherein L³ is as defined in claim 18;
wherein R⁵¹ is an amino protecting group as defined in claim 18;
wherein X³ is as defined in claim 20.

23. A process for the manufacture of a compound of formula (VII) comprising protection of the pyrrole nitrogen with a group R⁵¹, 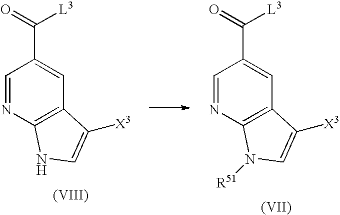
wherein L³ is as defined in claim 18;
wherein R⁵¹ is an amino protecting group defined in claim 18;
wherein X³ is as defined in claim 20.

24. A compound of formula (VIII) 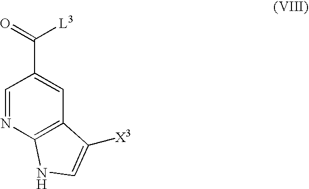
wherein L³ is as defined in claim 18;
and X³ is as defined in claim 20.

25. A process for the production of a compound of formula (VIII) by the introduction of an X³ group into a compound of formula (IX) 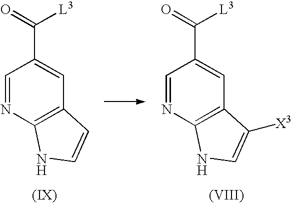
wherein L³ is as defined in claim 18 and X³ is as defined in claim 20.

26. A compound of formula (IX) 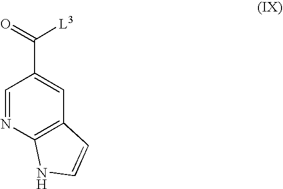
wherein L³ is a group L¹ as defined in claim 17 or a group R¹, wherein R¹ is a group of formula (II) 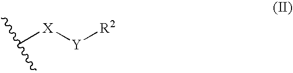
wherein X is NR³, O, S or (CR²²R²² )ₙ, Y is absent or is NR²³, O, or (CR²³R²³ )ₙ, R² is optionally substituted C₁₋₁₂ alkyl, C₂₋₁₂ alkenyl, C₂₋₁₂ alkynyl, C₃₋₁₂ carbocyclyl or C₃₋₁₂ heterocyclyl as claimed defined in any one of claims 1 to 14.

27. A process for the production of a compound of formula (VII) by the introduction of a X³group to a compound of formula (X) 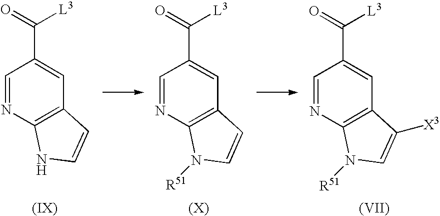
wherein L³ and R⁵¹ are as defined in claim 18 and X³ is as defined in claim 20.

28. A compound of formula (X) 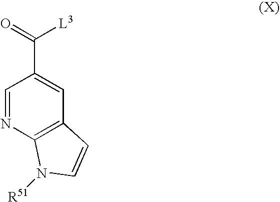
wherein L³ is a group L¹ as defined in claim 17 or a group R¹, wherein R¹ is a group of formula (II) 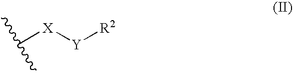
wherein X is NR³, O, S or (CR²²R²²)ₙ, Y is absent or is NR²³, O, or (CR²³R²³)ₙ, R² is optionally substituted C₋₁₂ alkyl, C₂₋₁₂ alkenyl, C₂₋₁₂ alkynyl, C₃₋₁₂ carbocyclyl or C₃₋₁₂ heterocyclyl as claimed defined in any one of claims 1 to 14;
and R⁵¹ is an amino protecting group as defined in claim 18.

29. A process for the preparation of compound of formula (IX) by the acid-catalysed hydrolysis of nitrile (XI) in the presence of an alcohol, 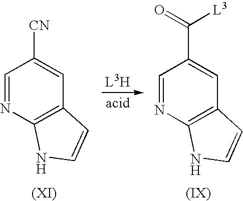
wherein L³ is OR⁵⁰;
and R⁵⁰ is as defined in claim 16.

30. A compound of formula (XI)

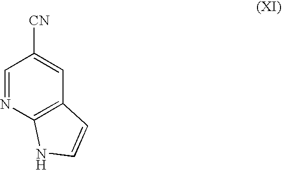

31. A process for the production of 1H-Pyrrolo[2,3-b]pyridine-5-carbonitrile (XI) comprising reaction of 5-bromo-1H-pyrrolo[2,3-b]pyridine with Zn(CN)₂ in the presence of a palladium catalyst.

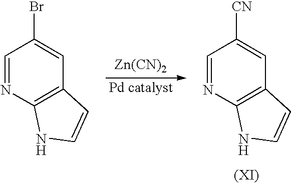

32. A compound as claimed in any one of claims 17, 19, 22, 24, 26, 28 or 30 selected from the group consisting of:

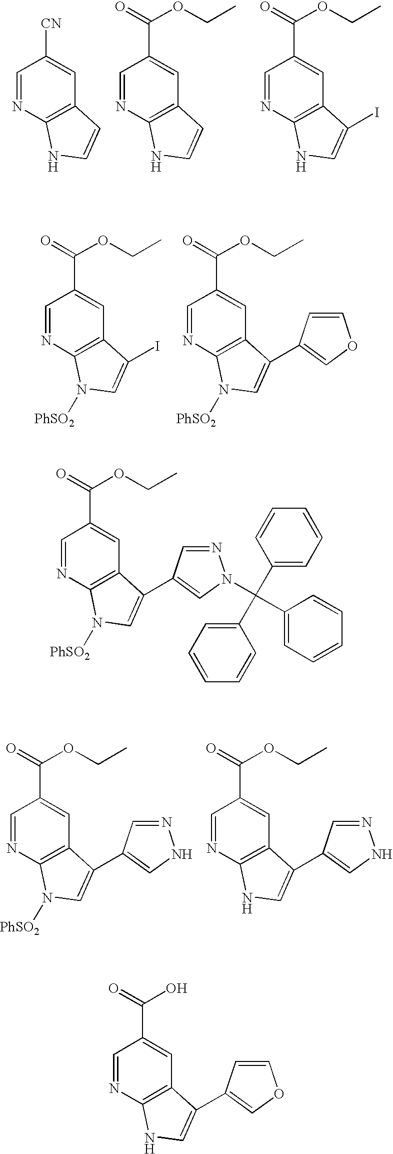

33. (canceled)

34. A pharmeceutical composition comprising a compound as claimed in claim 1 in combination with a pharmaceutically acceptable carrier, diluent or excipient.

35. A composition as claimed in claim 34, additionally comprising one or more of an anti-inflammatory agent, an AMPA receptor antagonist, a chemotherapeutic agent and/or an antiproliferative agent.

36. (canceled)

37. (canceled)

38. (canceled)

39. (canceled)

40. (canceled)

41. (canceled)

42. (canceled)

43. (canceled)

44. (canceled)

45. (canceled)

46. A method of treating or preventing a -mediated disorder in an individual, which method comprises administering to said individual a compound as claimed in claim 1 or a composition as claimed in claims 34 or 35.

47. (canceled)

48. A method as claimed in claim 46, wherein the disorder is a neurodegenerative disorder, inflammatory disease, a disorder linked to apoptosis, particularly neuronal apoptosis, autoimmune disease, destructive bone disorder, proliferative disorder, cancer, infectious disease, allergy, ischemia reperfusion injury, heart attack, angiogenic disorder, organ hypoxia, vascular hyperplasia, cardiac hypertrophy, thrombin induced platelet aggregation and/or any condition associated with prostaglandin endoperoxidase synthase-2.

49. A method as claimed in claim 48, wherein the neurodegenerative disorder results from apoptosis and/or inflammation.

50. A method as claimed in claim 48, wherein the neurodegenerative disorder is: dementia; Alzheimer's disease; Parkinson's disease; Amyotrophic Lateral Sclerosis; Huntington's disease; senile chorea; Sydenham's chorea; hypoglycemia; head and spinal cord trauma including traumatic head injury; acute and chronic pain; epilepsy and seizures; olivopontocerebellar dementia; neuronal cell death; hypoxia-related neurodegeneration; acute hypoxia; glutamate toxicity including glutamate neurotoxicity; cerebral ischemia; dementia linked to meningitis and/or neurosis; cerebrovascular dementia; or dementia in an HIV-infected patient.

51. A method as claimed in claim 48, wherein the neurodegenerative disorder is a peripheral neuropathy, including mononeuropathy, multiple mononeuropathy or polyneuropathy, such as may be found in diabetes mellitus, Lyme disease or uremia; peripheral neuiropathy caused hy a toxic agent; demyelinating disease such as acute or chronic inflammatory polyneuropathy, leukodystrophies or Guillain-Barré syndrome; multiple mononeuropathy secondary to a collagen vascular disorder; multiple mononeuropathy secondary to sarcoidosis; multiple mononeuropathy secondary to a metabolic disease, or multiple mononeuropathy secondary to an infectious disease.

52. A method as claimed in claim 46, wherein the disorder is inflammatory bowel disorder; bronchitis; asthma; acute pancreatitis; chronic pancreatitis; allergies of various types; Alzheimer's disease; autoimmune disease such as rheumatoid arthritis, systemic lupus erythematosus, glumerulonephritis, scleroderma, chronic thyroiditis, Graves's disease, autoimmune gastritis, diabetes, autoimmune haemolytis anaemia, autoimmune neutropaenia, thrombocytopenia, atopic dermatitis, chronic active hepatitis, myasthenia gravis, multiple sclerosis, ulcerative colitis, Crohn's disease, psoriasis or graft vs host disease.

53. A method as claimed in 46, wherein one or more other active agent is administered to the individual simultaneously, subsequently or sequentially to administering the compound.

54. A method as claimed in claim 53, wherein the other active agent is an anti-inflammatory agent.

55. (canceled)

56. (canceled)

57. (canceled)

58. (canceled)

59. (canceled)

60. (canceled)

61. (canceled)

62. (canceled)

63. An assay for determining the activity of the compounds as defined in claim 1, comprising providing a system for assaying the activity and assaying the activity of a compound as defined in claim 1.

64. An assay as claimed in claim 63, wherein the assay is for the JNK inhibiting activity of the compound, preferably for the JNK3-specific inhibiting activity of the compound.

65. An assay as claimed in claim 63, wherein the assay is a Scintillation Proximity Assay (SPA) using radiolabelled ATP, or is an ELISA.

66. A method of inhibiting the activity or function of a JNK, particularly JNK3, which method comprises exposing a JNK to a compound as defined in claim 1.

67. A method as claimed in claim 66, which is performed in a research model.

68. A method as claimed in claim 67, wherein the research model is an animal model.
